(19) United States
(12) Patent Application Publication
(10) Pub. No.: US 2024/0182596 A1
(43) Pub. Date: Jun. 6, 2024

(54) ANTI-NAPI2B ANTIBODIES AND ANTIBODY-DRUG-CONJUGATES BASED THEREON, THERAPEUTIC METHODS AND USES THEREOF

(71) Applicant: Tubulis GmbH, Planegg/Martinsried (DE)

(72) Inventors: Jonas Helma-Smets, München (DE); Annette Vogl, München (DE); Saskia Schmitt, München (DE); Isabelle Mai, München (DE); Sarah Herterich, München (DE); Dominik Schumacher, München (DE); Marc-André Kasper, München (DE); Philipp Cyprys, Deisenhofen (DE); Marcus Gerlach, München (DE)

(21) Appl. No.: 18/489,605

(22) Filed: Oct. 18, 2023

(30) Foreign Application Priority Data
Oct. 18, 2022 (EP) 22202150.3

(51) Int. Cl. C07K 16/30; A61K 47/68; A61P 35/00

(52) U.S. Cl. CPC C07K 16/3069; A61K 47/6869; A61P 35/00; C07K 2317/24; C07K 2317/92

(57) ABSTRACT

The present invention relates to novel anti-NaPi2b antibodies, antibody-drug-conjugates (ADCs) based thereon as well as to therapeutic methods and uses thereof, particularly in relation to cancer treatment.

### CROSS-REFERENCE TO RELATED APPLICATIONS

The present application claims the right of priority of European patent application EP22202150.3 filed with the European Patent Office on 18 Oct. 2022, the entire content of which is incorporated herein for all purposes.

### REFERENCE TO AN ELECTRONIC SEQUENCE LISTING

The contents of the electronic sequence listing (910271_403_SeqListing_V2.xml; Size: 90,560 bytes; and Date of Creation: Oct. 18, 2023) is herein incorporated by reference in its entirety.

### TECHNICAL FIELD OF THE INVENTION

The present invention relates to novel anti-NaPi2b antibodies, antibody-drug-conjugates (ADCs) based thereon as well as to therapeutic methods and uses thereof, particularly in relation to cancer treatment.

### BACKGROUND OF THE INVENTION

NaPi2b, encoded by the SLC34A2 gene, is a multitransmembrane, sodium-dependent phosphate transporter, which is expressed in human lung, ovarian, and thyroid carcinomas, as well as the normal tissues from which these tumors arise. As a member of the SLC34 solute carrier protein family, it is responsible for transcellular inorganic phosphate absorption and maintenance of phosphate homeostasis and has been associated with cell differentiation and tumorigenesis. Napi2b mRNA/protein expression has been described in nonsquamous non-small cell lung carcinoma, nonmucinous ovarian carcinoma, and papillary thyroid carcinoma; normal tissue expression has been reported in lung, bronchus, and kidney (Lin et al., 2015). In normal lung tissue, NaPi2b plays a role in phosphate transport, and mutations in SLC34A2 are associated with pulmonary and testicular microlithiasis (Corut et al., 2006). The differential expression in tumor relative to most normal tissues, prominent cell-surface localization, and high endocytosis rates, make Napi2b a promising target for ADC therapeutics. Several first-generation antibody-drug conjugates have also been developed in the past, which however either not result in long-term survival benefits or are associated with dose-limiting toxiciticities.

Accordingly, there is a need for novel antibodies with improved characteristics and ADCs based thereon that have improved toxicity profiles with greater functionality. The technical problem of the present invention is therefore to comply with this need.

The present invention complies with that need inter alia by providing novel anti-NaPi2b antibodies having reduced tendency for antibody modifications by post-translational- or post expression and purification modifications (e.g. deamidation), improved on-target binding, improved target dependend internalization rates, limited tendency for aggregation and HMWS formation as well as cross-reactivity with rat Napi2b and cynomolgus monkey NaPi2b with a similar binding capacity compared to human Napi2b, which allows for better toxicity analysis and comparability, and novel NaPi2b-targeting antibody-drug-conjugates (ADCs) based thereon, which were generated by applying the P5 conjugation technology (e.g., W02018/041985), which is based on the modification of interchain-Cysteine residues with unsaturated phosphonamidate reagents, as well as therapeutic methods and uses thereof, particularly in relation to cancer treatment.

### SUMMARY OF INVENTION

The technical problem is solved by the subject-matter as defined in the claims.

Accordingly, the present invention relates to an anti-NaPi2b antibody (e.g., an antibody against NPT2B_HUMAN Sodium-dependent phosphate transport protein 2B, e.g., having UniProt Accession Number: 095436 or SEQ ID NO: 1 and/or an antibody against NPT2B_RAT Sodium-dependent phosphate transport protein 2B, e.g., having UniProt Accession Number: Q9JJ09 or SEQ ID NO: 2), wherein said NaPi2b antibody is capable of the following: (a) binding to human Napi2b (e.g., SEQ ID NO: 1) and/or rat Napi2b (e.g., SEQ ID NO: 2), preferably said binding to said human Napi2b and said rat Napi2b having about the same K_{D}, further preferably said antibody is selected from the group consisting of: AV-25, AV-15, AV-18, AV-21 and AV-29 antibody), further most preferably said about same K_{D} having up to 50% difference (e.g., up to 45%, 40%, 35%, 30%, 25%, 20%, 15%, 10%, 9%, 8%, 7%, 6%, 7%, 5%, 4%, 3%, 2% or 1% difference); (b) cross-reactivity with rat Napi2b (e.g., having UniProt Accession Number: Q9JJ09 or SEQ ID NO: 2), (c) cross-reactivity with cynomolgus monkey (e.g., Macaca fascicularis) Napi2b (e.g., having UniProtKB Accession Number: A0A2K5UHY1 or SEQ ID NO: 3; (d) internalization, preferably by the means of the antigen-mediated antibody internalization; (e) optionally, not having a dipeptide deamidation site in a heavy chain variable region's (V_{H}) CDR2, preferably said absent dipeptide deamidation site is NG (Asn-Gly) in V_{H} CDR2.

The present invention further relates to monoclonal human or humanized IgG1 anti- Napi2b antibody of the present invention, preferably comprising kappa (κ) light chain.

The present invention further relates to a hybridoma, wherein said hybridoma produces the antibody of the present invention.

The present invention further relates to a nucleic acid encoding the antibody of the present invention.

The present invention further relates to an expression vector comprising at least one of the nucleic acid molecules of the present invention.

The present invention further relates to an isolated host cell (e.g., an isolated recombinant host cell) comprising the vector and/or nucleic acid of the present invention.

The present invention further relates to an antibody drug conjugate (ADC) comprising the anti-Napi2b antibody of the present invention.

The present invention further relates to an antibody drug conjugate (ADC) of the present invention comprising the anti-NaPi2b antibody of the present invention (e.g., humanized monoclonal NaPi2b-specific IgG1 antibody) conjugated to a cytotoxic payload/drug: (a) wherein the cytotoxic pyload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or (b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or (c) wherein the cytotoxic pyload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or (d) wherein the linker (L) comprises a valine-citrulline-PAB moiety or a valine-alanine-PAB moiety; and/or (e) wherein cytotoxic payload is Exatecan, conjugated via a chemical valine-citrulline-PAB or a valine-alanine-PAB release unit, wherein said release unit is cleavable by a protease. Preferably, a drug (e.g., cytotoxic moieties (e.g., cytotoxic payload/s, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan) to antibody ratio (DAR) is in the range between 0 and 20; further preferably DAR is in the range from 4 to 8, most preferably DAR is 4 or 8.

The present invention further relates to a composition or kit comprising the anti- NaPi2b, antibody drug conjugate (ADC), hybridoma, nucleic acid, expression vector and/or host cell of the present invention.

The present invention further relates to method of synthesis of the antibody drug conjugates (ADCs) of the present invention.

The present invention further relates to methods of treatment and uses of the antibody, antibody drug conjugate (ADC), nucleic acid, expression vector, host cell, composition and/or kit of the present invention.

### OVERVIEW OF THE SEQUENCE LISTING

As described herein and unless otherwise stated references are made to UniProtKB Accession Numbers (https://www.uniprot.org/release-notes/2022-08-03-release, e.g., as available in UniProt release 2022_03, published Aug. 3, 2022).

This application contains a Sequence Listing in computer readable form, which is incorporated herein by reference.

### BRIEF DESCRIPTION OF THE DRAWINGS

The invention will be better understood with reference to the detailed description when considered in conjunction with the non-limiting examples and the accompanying drawings, respectively. The Figures show:

FIG. 1 shows exemplary sequences of the anti-NaPi2b antibodies of the present invention.

FIG. 2 shows Charge-Variant analysis of the exemplary antibodies of the present invention.

FIGS. 3A to 3D show binding to human and rat NaPi2B evaluated by Flow Cytometry. Graph shows means of n=2±SEM.

FIG. 4 shows binding to immobilized human NaPi2B evaluated by ELISA. Graph shows means of n=2±SEM.

FIG. 5 shows Internalization evaluated via flow cytometry.

FIG. 6 shows melting curves evaluated by NanoDSF.

FIG. 7 shows in vitro cytotoxicity evaluated via Resazurin assay in NaPi2B-positive cells. Graph shows means of n=2±SEM.

FIG. 8 shows bystander activity of the ADC.

FIG. 9 shows in vitro inhibition of Topoisomerase-I, by the delivery of Exatecan via the ADC.

FIG. 10 shows in vivo PK Evaluation of the ADC and the unconjugated antibody.

FIG. 11 shows the UV chromatogram of the LC/MS measurement of P5(PEG12)-COOH.

FIG. 12 shows shows the UV chromatogram of the LC/MS measurement of P5(PEG24)-OSu.

FIG. 13 shows shows the UV chromatogram of the LC/MS measurement of NH2-VC-PAB-Exatecan TFA salt.

FIG. 14 shows shows the UV chromatogram of the LC/MS measurement of NH2-VA-PAB-Exatecan TFA salt.

FIG. 15 shows shows the UV chromatogram of the LC/MS measurement of P5(PEG2)-VC-PAB-Exatecan.

FIG. 16 shows shows the UV chromatogram of the LC/MS measurement of P5(PEG12)-VC-PAB-Exatecan.

FIG. 17 shows shows the UV chromatogram of the LC/MS measurement of P5(PEG24)-VC-PAB-Exatecan.

FIG. 18 shows shows the UV chromatogram of the LC/MS measurement of P5(PEG12)-VA-PA B-Exatecan.

FIG. 19 shows the UV chromatogram of the LC/MS measurement of P5(PEG12)-Exatecan.

FIG. 20 shows analytical characterization of synthesized mAbs and DAR8-ADCs from P5(PEG24)-VC-PAB-Exatecan.

FIGS. 21A to 21D show analytical Raw Data for AV25. Analytical characterization of one of the described mAbs. The antibody was expressed in Expi-CHO cells and purified via Protein A chromatography, as described above. The mAb has been analyzed via HLPC-SEC (FIG. 21A), LC-MS (FIG. 21B), HLPC-HIC (FIG. 21C) and reducing SDS-PAGE (FIG. 21D).

FIG. 22 shows LC/MS analysis of the parental antibody.

FIG. 23 shows LC/MS analysis of AV15.

FIG. 24 shows LC/MS analysis of AV18.

FIG. 25 shows LC/MS analysis of AV21.

FIG. 26 shows LC/MS analysis of AV25.

FIG. 27 shows LC/MS analysis of AV29.

FIGS. 28A to 28C show analytical Raw Data for AV25-P5(PEG24)-VC-PAB-Exatecan DAR8. Analytical characterization of one of the ADCs, synthesized and purified as described above. A) Analytical size-exclusion chromatography (FIG. 28A), B) LC-MS of the ADC product (FIG. 28B), C) analytical hydrophobic interaction chromatography after conjugation (FIG. 28C). The data indicates that the ADC is fully conjugated to DAR8 with only low amounts of aggregates being present after purification.

FIG. 29 shows LC/MS analysis of the Parental-P5(PEG24)-VC-PAB-Exatecan DAR8

FIG. 30 shows LC/MS analysis of an exemplary ADC of the present invention, AV15-P5(PEG24)-VC-PAB-Exatecan (DAR8).

FIG. 31 shows LC/MS analysis of an exemplary ADC of the present invention, AV18-P5(PEG24)-VC-PAB-Exatecan (DAR8).

FIG. 32 shows LC/MS analysis of an exemplary ADC of the present invention, AV21-P5(PEG24)-VC-PAB-Exatecan (DAR8).

FIG. 33 shows LC/MS analysis of an exemplary ADC of the present invention, AV25-P5(PEG24)-VC-PAB-Exatecan (DAR8).

FIG. 34 shows LC/MS analysis of an exemplary ADC of the present invention, AV29-P5(PEG24)-VC-PAB-Exatecan (DAR8).

FIG. 35 shows results of an ex vivo serum stability analysis of AV25-P5(PEG24)-VC-PAB-Exatecan (DAR 8), which is a representative ADC of the present invention.

FIG. 36 shows results of an in vivo efficacy analysis of the AV25-P5(PEG24)-VC-PAB-Exatecan (DAR 8), which is a representative ADC of the present invention, in a cell line derived xenograft model (CDX).

FIG. 37 shows results of an in vivo efficacy analysis of the AV25-P5(PEG24)-VC-PAB-Exatecan (DAR 8), which is a representative ADC of the present invention, in patient-derived xenograft models (PDX).

FIG. 38 shows results of an in vivo toxicity analysis of the AV25-P5(PEG24)-VC-PAB-Exatecan (DAR 8), which is a representative ADC of the present invention, in cynomolgus monkeys.

FIG. 39 shows a MS analysis of the anti-Napi2b comparison ADC, synthesized by the method described above. Signals are annotated with the measured mass in Dalton and the absolute intensities. Shown is an exemplary spectrum from the conjugation reaction. The Drug-to-Antibody ratio of the final conjugated have been estimated from the MS signals to 3.5 to 4.0. LC: light chain of anti-Napi2b comparison antibody, HC: heavy chain anti-Napi2b comparison antibody.

FIG. 40 shows binding, expressed as MFI ratio, of unmodified AV25 mAb and AV25-P5(PEG24)-VC-PAB-Exatecan DAR8 ADC to HEK293-cells transfected with Napi2a (left), Napi2b (middle) and Napi2c (right). AV25 mAb and AV25-P5(PEG24)-VC-PAB-Exatecan DAR8 ADC bind specifically to NaPi2b. Graphs show n=2±SD (note, error bars are too small to be displayed).

FIG. 41 shows the cytotoxicity dose-response of AV25-P5(PEG24)-VC-PAB-Exatecan DAR 8 compared to anti-Napi2b comparison ADC on 3 different cell lines. Shown are mean and SD of two measurements, as well as a dose-response-fit. Fluorescence in % of medium control is equal to % of viable cells.

FIGS. 42A and 42B show the dose-dependent induction of DNA-damage and apoptosis markes in response to treatment with increasing concentrations of AV25-P5(PEG24)-VC-PAB-Exatecan DAR8. A corresponding non-targeting isotype control conjugate was included as negative control. HCC-78 cells (FIG. 42A, NaPi2b^{high}) and OVCAR-3 cells (FIG. 42B, NaPi2b^{high}) were treated with increasing concentrations (0.05-12 μg/ml) of AV25-P5(PEG24)-VC-PAB-Exatecan DAR8 or the Isotype ADC (Isotype-P5(PEG24)-VC-PAB-Exatecan DAR8) for 72h, Cells were stained for cleaved PARP (left), Caspase 3 (middle) and pH2AX (right) and analyzed via flow cytometry. Graphs show means of n=2±SEM.

FIG. 43 shows binding of the Fc region of the AV25 HC-LALA antibody versus the AV25 HC-wt antibody to recombinant hexameric C1q complement protein which was measured in a HTRF (Homogenous Time-Resolved Fluorescence) based human C1q binding assay (HTRF Human C1q Binding Kit, Cisbio) according to manufacturer's instructions. In brief, serial dilutions 280 nM - 11.6 nM of all tested antibodies (AV25 HC-wt, AV25 HC-LALA, α-MHC-I positive, IgG1 kit standard) were measured. HTRF ratio was calculated by dividing the acceptor emission signal at 665 nm by the donor emission signal at 620 nm and multiplying by 10000. Graph shows HTRF ratio at 70 nM concentration with the background (diluent only) HTRF ratio subtracted. Graph shows means of n=2±SD.

FIGS. 44A to 44D show dose-dependent binding of the Fc region of the AV25-HC-LALA antibody and the AV25-P5(PEG24)-VC-PAB-Exatecan DAR8 ADC versus the AV25-HC-wt antibody to recombinant human FcRn and FcγR which was measured using Lumit™ FcγR Binding Immunoassays (FcγRn, FcγRI, FcγRIIa/CD32 131/H131 polymorphism, FcγRIIIa/CD16 V158/F158 polymorphism; Promega) according to the manufacturer's instructions. Serial dilutions of AV25 HC-wt, AV25 HC-LALA and AV25-P5(PEG24)-VC-PAB-Exatecan DAR8, a standard and trastuzumab as positive control were incubated with the Tracer-LgBiT and a FcγR-SmBiT. In the absence of an antibody analyte or if no interaction of the tested antibody with FcγRs takes place, Tracer-LgBiT binds to the FcγR-SmBiT target, resulting in maximum luminescence signal. In the case of successful interaction with FcγRs, the tested antibody/ADC will compete with Tracer-LgBiT for binding to the FcγR target, resulting in a concentration-dependent decrease in the luminescent signal. Luminescence was measured on a microplate reader Infinite M200 Pro (Tecan). Graphs show n=1.

FIG. 45 shows a calcein release-based antibody-dependent cellular cytotoxicity (ADCC) assay. Co-cultures of healthy donor (HD)-derived NK cells and calcein-stained target-positive tumor cells (OVCAR-3 and HCC-78), in a 4:1 ratio, were incubated with 15 μg/ml of indicated antibodies or ADCs (anti-MHC-I antibody served as positive control). The percentage of specific killing was calculated by deviding the calcein released by antibody-mediated cell killing from calcein released by Triton X-permeabilized cells (maximum killing). Graphs show means of n=2±SEM.

FIG. 46 shows results of an in vivo efficacy analysis of the AV25-P5(PEG24)-VC-PAB-Exatecan (DAR 8), which is a representative ADC of the present invention, in a patient-derived non-small cell lung cancer (NSCLC) xenograft model (PDX, Lu7700). Shown on the left is tumor volume over time after treatment once at day 0 with different dose levels of the AV25-P5(PEG24)-VC-PAB-Exatecan (DAR 8) (1, 3 and 5 mg ADC/kg bodyweight), an isotype control carrying the same amount of linker-payload (5 mg/kg) versus untreated (vehicle). Shown on the right are bodyweights of the animals treated with different dose levels of the AV25-P5(PEG24)-VC-PAB-Exatecan (DAR 8), an isotype control carrying the same amount of linker-payload versus untreated (vehicle). All results are shown as Mean and SEM of 4 animals per group.

FIG. 47A shows the results of an in vivo efficacy analysis of the AV25-P5(PEG24)-VC-PAB-Exatecan DAR 8, which is a representative ADC of the present invention, in a patient-derived ovarian cancer xenograft model (PDX, Ov6668). Shown on the left is tumor volume over time after treatment once at day 0 with different dose levels of the AV25-P5(PEG24)-VC-PAB-Exatecan (DAR 8) (1, 3 and 5 mg ADC/kg bodyweight) versus untreated (vehicle). Shown on the right are bodyweights of the animals treated with different dose levels of the AV25-P5(PEG24)-VC-PAB-Exatecan (DAR 8), versus untreated (vehicle). All results are shown as Mean and SEM of 9 animals per group. FIG. 47B shows the in vivo PK evaluation for total and intact ADC for the three dose levels of the dose-response efficacy study as Mean and SD from three measurements per time point from mice treated once at day 0 with different dose levels of the AV25-P5(PEG24)-VC-PAB-Exatecan (1, 3 and 5 mg ADC/kg bodyweight). PK evaluation has been performed as described in example 1.

FIG. 48 shows results of an in vivo efficacy analysis of the AV25-P5(PEG24)-VC-PAB-Exatecan (DAR 8), which is a representative ADC of the present invention, in patient-derived ovarian cancer xenograft models. Shown on top is tumor volume over time after treatment once at day 0 with 10 mg ADC/kg bodyweight of the AV25-P5(PEG24)-VC-PAB-Exatecan (DAR 8) or Isotype- P5(PEG24)-VC-PAB-Exatecan (DAR 8) versus untreated (vehicle). Shown on the bottom is a BRCA-mutated model with low NaPi2B expression (bottom left) and high NaPi2B expression (bottom right). All results are shown as Mean and SEM of 3 animals per group.

FIG. 49 shows dose-dependent binding, expressed as MFI ratio, of increasing concentraions of Upifitamab or AV25 normalized to the non-specific binding control on two different NaPi2b-positive cell-lines (OVCAR-3, left, HCC78, right). Shown are mean and SD of two measurements, as well as a dose-response-fit.

FIG. 50 shows cytotoxicity dose-response of AV25-P5(PEG24)-VC-PAB-Exatecan DAR 8 compared to Upifitamab-P5(PEG24)-VC-PAB-Exatecan DAR 8 and an isotype control Isotype-P5(PEG24)-VC-PAB-Exatecan DAR 8 on two different cell lines. Shown are mean and SD of two measurements, as well as a dose-response-fit. Fluorescence in % of medium control is equal to % of viable cells.

### DETAILED DESCRIPTION OF THE INVENTION

The present invention is described in detail in the following and is also illustrated by the appended examples and figures.

The present inventors produced and characterized novel specific anti-NaPi2b antibodies for specifically targeting the extracellular domain of NaPi2b. This is particularly advantageous as it relates to a new therapeutic method for treating cancer (e.g., preferably said cancer is a solid and/or metastatic cancer, further preferably said cancer is selected from the group consisting of: lung cancer, ovarian cancer, thyroid cancer, nonsquamous non-small cell lung carcinoma, nonmucinous ovarian carcinoma, papillary thyroid carcinoma, renal cancer, endometrial cancer, uterus cancer, ureter cancer, bladder cancer and fallopian tube cancer).

The present invention provides novel anti-NaPi2b antibodies having reduced tendency for antibody modifications by post-translational modifications (e.g. deamidation), improved on-target binding, improved target dependend internalization rates, limited tendency for aggregation and HMWS formation as well as cross-reactivity with rat Napi2b and cynomolgus monkey NaPi2b with a similar binding capacity compared to human Napi2b, which allows for better toxicity analysis and comparability, and novel NaPi2b-targeting antibody-drug-conjugates (ADCs) based thereon, which were generated by applying the P5 conjugation technology (e.g., W02018/041985), which is based on the modification of interchain-Cysteine residues with unsaturated phosphonamidate reagents, as well as therapeutic methods and uses thereof, particularly in relation to cancer treatment.

The novel antibodies of the present invention are humanized anti-NaPi2b monoclonal antibody (mAb) optionally comprising Fc silencing mutations such as leucine (L) to alanine (A) substitution at the position 234 and 235 (LALA mutations), binding to human Napi2b (e.g., SEQ ID NO: 1) and/or rat Napi2b (e.g., SEQ ID NO: 2), having cross-reactivity with rat and cynomolgus monkey Napi2b and optionally, not having a dipeptide deamidation site in a heavy chain variable region's (V_{H}) CDR2, preferably said absent dipeptide deamidation site is NG (Asn-Gly) in V_{H} CDR2.

The antibody drug conjugates (ADCs) of the present invention comprising an anti-NaPi2b antibody of the present invention (e.g., an antibody against NPT2B_HUMAN Sodium-dependent phosphate transport protein 2B, e.g., having UniProt Accession Number: 095436 or SEQ ID NO: 1 and/or an antibody against NPT2B_RAT Sodium-dependent phosphate transport protein 2B, e.g., having UniProt Accession Number: Q9JJ09 or SEQ ID NO: 2), wherein said NaPi2b antibody is capable of the following: (a) binding to human Napi2b (e.g., SEQ ID NO: 1) and/or rat Napi2b (e.g., SEQ ID NO: 2), preferably said binding to said human Napi2b and said rat Napi2b having about the same K_{D}, further preferably said antibody is selected from the group consisting of: AV-25, AV-15, AV-18, AV-21 and AV-29 antibody), further most preferably said about same K_{D} having up to 50% difference (e.g., up to 45%, 40%, 35%, 30%, 25%, 20%, 15%, 10%, 9%, 8%, 7%, 6%, 7%, 5%, 4%, 3%, 2% or 1% difference); (b) cross-reactivity with rat Napi2b (e.g., having UniProt Accession Number: Q9JJ09 or SEQ ID NO: 2), (c) cross-reactivity with cynomolgus monkey (e.g., Macaca fascicularis) Napi2b (e.g., having UniProtKB Accession Number: A0A2K5UHY1 or SEQ ID NO: 3; (d) internalization, preferably by the means of the antigen-mediated antibody internalization; (e) optionally, not having a dipeptide deamidation site in a heavy chain variable region's (V_{H}) CDR2, preferably said absent dipeptide deamidation site is NG (Asn-Gly) in V_{H} CDR2; wherein said anti-NaPi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) cytotoxic moieties (e.g., cytotoxic payloads, e.g., Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan), preferably via one or more linkers, further preferably via one or more phosphonamidate linkers.

### Definitions

### Antibodies

An “antibody” when used herein may refer to a protein comprising one or more polypeptides (comprising one or more binding domains and/or antigen binding portion, preferably antigen binding domains) substantially or partially encoded by immunoglobulin genes or fragments of immunoglobulin genes. The term “immunoglobulin” (Ig) is used interchangeably with “antibody” herein. The recognized immunoglobulin genes include the kappa, lambda, alpha, gamma, delta, epsilon and mu constant region genes, as well as myriad immunoglobulin variable region genes. In particular, an “antibody” when used herein, is typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2, with IgG being preferred in the context of the present invention. An antibody of the present invention is also envisaged which has an IgE constant domain or portion thereof that is bound by the Fc epsilon receptor I. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each light chain includes an N-terminal variable (V) domain (VL) and a constant (C) domain (CL). Each heavy chain includes an N-terminal V domain (VH), three or four C domains (CHs), and a hinge region. The constant domains are not involved directly in binding an antibody to an antigen, but can exhibit various effector functions, such as participation of the antibody dependent cellular cytotoxicity (ADCC). If an antibody should exert ADCC, it is preferably of the IgG1 subtype, while the IgG4 subtype would not have the capability to exert ADCC.

The term “antibody” also includes, but is not limited to, but encompasses monoclonal, monospecific, poly- or multi-specific antibodies such as bispecific antibodies, humanized, camelized, human, single-chain, chimeric, synthetic, recombinant, hybrid, mutated, grafted, and in vitro generated antibodies, with chimeric or humanized antibodies being preferred. The term “humanized antibody” is commonly defined for an antibody in which the specificity encoding CDRs of HC and LC have been transferred to an appropriate human variable frameworks (“CDR grafting”). The term “antibody” also includes scFvs, single chain antibodies, diabodies or tetrabodies, domain antibodies (dAbs) and nanobodies. In terms of the present invention, the term “antibody” shall also comprise bi-, tri- or multimeric or bi-, tri- or multifunctional antibodies having several antigen binding sites.

Furthermore, the term “antibody” as employed in the invention also relates to derivatives of the antibodies (including fragments) described herein. A “derivative” of an antibody comprises an amino acid sequence which has been altered by the introduction of amino acid residue substitutions, deletions or additions. Additionally, a derivative encompasses antibodies which have been modified by a covalent attachment of a molecule of any type to the antibody or protein. Examples of such molecules include sugars, PEG, hydroxyl-, ethoxy-, carboxy- or amine-groups but are not limited to these. In effect the covalent modifications of the antibodies lead to the glycosylation, pegylation, acetylation, phosphorylation, amidation, without being limited to these.

The antibody of the present invention is preferably an “isolated” antibody. “Isolated” when used to describe antibodies disclosed herein, means an antibody that has been identified, separated and/or recovered from a component of its production environment. Preferably, the isolated antibody is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody will be prepared by at least one purification step.

Antibodies described herein can be used for diagnostic purposes, including sample testing and in vivo imaging, and for this purpose the antibody (or binding fragment thereof) can be conjugated to an appropriate detectable agent, to form an immunoconjugate. For diagnostic purposes, appropriate agents are detectable labels that include radioisotopes, for whole body imaging, and radioisotopes, enzymes, fluorescent labels and other suitable antibody tags for sample testing. The detectable labels can be any of the various types used currently in the field of in vitro diagnostics, including particulate labels including metal sols such as colloidal gold, isotopes, chromophores including fluorescent markers, biotin, luminescent markers, phosphorescent markers and the like, as well as enzyme labels that convert a given substrate to a detectable marker, and polynucleotide tags that are revealed following amplification such as by polymerase chain reaction. A biotinylated antibody would then be detectable by avidin or streptavidin binding. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-stare or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

Antibody “effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptors); and B cell activation. In order to exert effector functions an antibody, so to say, recruits effector cells.

As used herein the term “antigen binding portion” refers to a fragment of immunoglobulin (or intact antibody), and encompasses any polypeptide comprising an antigen-binding fragment or an antigen-binding domain. Preferably, the fragment such as Fab, F(ab′), F(ab′)₂, Fv, scFv, Fd, disulfide-linked Fvs (sdFv), and other antibody fragments that retain antigen-binding function as described herein. Typically, such fragments would comprise an antigen-binding domain and have the same properties as the antibodies described herein. Accordingly, said fragment is preferably also capable of binding to an extracellular domain of the NaPi2b.

As used herein, the term “specifically binds” refers to antibodies or fragments or derivatives thereof that specifically bind to NaPi2b protein and do not specifically bind to another protein. The antibodies or fragments or derivatives thereof according to the invention bind to a NAPI2B protein through the variable domain of the antibody.

The pairing of a VH and VL together forms a single antigen-binding site. The CH domain most proximal to VH is designated as CH1. Each L chain is linked to an H chain by one covalent disulfide bond, while the two H chains are linked to each other by one or more disulfide bonds depending on the H chain isotype. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1 or H-CDR1 (or CRD-H1), H2 or H-CDR2 (or CDR-H2) and H3 or H-CDR3 (or CDR-H3), while CDR constituents on the light chain are referred to as L1 or L-CDR1 (or CRD-L1), L2 or L-CDR2 (or CDR-L2), and L3 or L-CDR3 (or CDR-L3).

The term “variable” refers to the portions of the immunoglobulin domains that exhibit variability in their sequence and that are involved in determining the specificity and binding affinity of a particular antibody (i.e., the “variable domain(s)”). Variability is not evenly distributed throughout the variable domains of antibodies; it is concentrated in sub-domains of each of the heavy and light chain variable regions. These sub-domains are called “complementarity determining regions” (CDRs).

The terms “CDR”, and its plural “CDRs”, refer to a complementarity determining region (CDR) of which three make up the binding character of a light chain variable region (L1-CDR, L2-CDR and L3-CDR) and three make up the binding character of a heavy chain variable region (H1-CDR, H2-CDR and H3-CDR). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. However, the numbering in accordance with the so-called Kabat system is preferred.

The more conserved (i.e., non-hypervariable) portions of the variable domains are called the “framework” regions (FRM). The variable domains of naturally occurring heavy and light chains each comprise four FRM regions, largely adopting a β-sheet configuration, connected by three hypervariable regions, which form loops connecting, and in some cases forming part of, the R -sheet structure. The hypervariable regions in each chain are held together in close proximity by the FRM and, with the hypervariable regions from the other chain, contribute to the formation of the antigen- binding site (see Kabat et al., loc. cit.). The constant domains are not directly involved in antigen binding, but exhibit various effector functions, such as, for example, antibody-dependent, cell-mediated cytotoxicity and complement activation.

The term “binding domain” characterizes in connection with the present invention a domain of a polypeptide which specifically binds/interacts with a given target epitope. An “epitope” is antigenic and thus the term epitope is sometimes also referred to herein as “antigenic structure” or “antigenic determinant”. Thus, the binding domain is an “antigen-interaction-site”. The term “antigen-interaction-site” defines, in accordance with the present invention, a motif of a polypeptide, which is able to specifically interact with a specific antigen or a specific group of antigens, e.g. the identical antigen in different species. Said binding/interaction is also understood to define a “specific recognition”.

The terms “antigen-binding domain”, “antigen binding portion”, “antigen-binding fragment” and “antibody binding region” when used herein refer to a part of an antibody molecule that comprises amino acids responsible for the specific binding between antibody and antigen. The part of the antigen that is specifically recognized and bound by the antibody is referred to as the “epitope” as described herein above. As mentioned above, an antigen-binding domain may typically comprise an antibody light chain variable region (VL) and an antibody heavy chain variable region (VH).

The term “epitope” also refers to a site on an antigen (in the context of the present invention, the antigen is NaPi2b protein) to which the antibody molecule binds. Preferably, an epitope is a site on a molecule (in the context of the present invention, the antigen is a NaPi2b protein) against which a antibody or antigen binding portion thereof, preferably an antibody will be produced and/or to which an antibody will bind. For example, an epitope can be recognized by a antibody or antigen binding portion thereof. A “linear epitope” is an epitope where an amino acid primary sequence comprises the epitope recognized. A linear epitope typically includes at least 3, and more usually, at least 5, for example, about 8 to about 10 amino acids in a unique sequence.

The term “cross reactivity” may refer to the ability of an antibody to react with similar antigenic sites on different proteins.

The term “specifically” in this context may mean that the antibody or antigen binding portion thereof binds to target NaPi2b, but does not binds to another protein. The term “another protein” includes any protein including proteins closely related to or being homologous to NAPI2B protein against which the antibody or antigen binding portion thereof is directed to. However, the term “another protein” does not include that the antibody or antigen binding portion thereof cross-reacts with NaPi2b protein from another species different from that against which the antibody or antigen binding portion thereof was generated.

Thus, cross-species specific antibody or antigen binding portion thereof directed against NaPi2b protein are preferably contemplated by the present invention.

The term “K_{D}” may refer to the equilibrium dissociation constant, a ratio of k_{off}/kₒₙ, between the antibody and its antigen or between the variable regions of one heavy and one light chain of an antibody or fragment or derivative thereof and their antigen (e.g., Napi2b, e.g., full-length Napi2b and/or one or more fragments thereof, preferably said one or more fragments comprising at least one extracellular domain (ECD) of said Napi2b (e.g., said ECD comprising amino acids 122-135 and/or amino acids 235-361 and/or 429-485 and/or amino acids 547-552 of the human Napi2b having SEQ ID NO: 1) and/or one or more fragments of said ECD (e.g., having length from about 15 to about 30 amino acids), e.g., said full-length Napi2b and/or one or more fragments thereof are fused or not fused to one or more protein tags (e.g., 6 x His-tags, FLAG, HA, V5, Fc-fusion, MBP, SUMO, TEV, GFP, TST) and is measured in vitro. K_{D} and affinity are inversely related.

As used herein, the term “affinity” may refer to the binding strength between the variable regions of one heavy and one light chain of an antibody or fragment or derivative thereof and their antigen (e.g., Napi2b, e.g., full-length Napi2b and/or one or more fragments thereof, preferably said one or more fragments comprising at least one extracellular domain (ECD) of said Napi2b (e.g., said ECD comprising amino acids 122-135 and/or amino acids 235-361 and/or 429-485 and/or amino acids 547-552 of the human Napi2b having SEQ ID NO: 1) and/or one or more fragments of said ECD (e.g., having length from about 15 to about 30 amino acids e.g., said full-length Napi2b and/or one or more fragments thereof are fused or not fused to one or more protein tags (e.g., 6 x His-tags, FLAG, HA, V5, Fc-fusion, MBP, SUMO, TEV, GFP, TST) and is measured in vitro. Affinity determines the strength of the interaction between an epitope and an antibody's antigen binding site. Affinity can be calculated using the following formula:

KA=[AB−AG]/[AB]*[AG]=kₒₙ/k_{off}
wherein:
KA=affinity constant
[AB]=molar concentration of unoccupied binding sites on the antibody
[AG]=molar concentration of unoccupied binding sites on the antigen
[AB-AG]=molar concentration of the antibody-antigen complex

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gin, Gly, His, Met, Phe, Ser, Thr, Trp, and Tyr).

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is effected e.g. by post-translational modifications like glycosylation, acetylation, phosphorylation and the like. Such modifications are well known in the art.

The term “immune cells” refers to cells which are capable of producing antibodies. The immune cells of particular interest herein are lymphoid cells derived, e.g. from spleen, peripheral blood lymphoctes (PBLs), lymph node, inguinal node, Peyers patch, tonsil, bone marrow, cord blood, pleural effusions and tumor-infiltrating lymphocytes (TIL).

A type of antibody variant encompassed by the present invention is an amino acid substitution variant. These variants have at least one, two, three, four, five, six, seven, eight, nine or ten amino acid residues in the Napi2b antibody molecule replaced by a different residue. The sites of greatest interest for substitutional mutagenesis include the CDRs of the heavy and/or light chain, in particular the hypervariable regions, but FR alterations in the heavy and/or light chain are also contemplated.

For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

Generally, if amino acids are substituted in one or more or all of the CDRs of the heavy and/or light chain, it is preferred that the then-obtained “substituted” sequence is at least 60% (e.g., at least 65%, at least 70%, at least 75%, at least 80%, at least 85%, at least 90%, at least 95%, at least 96%, at least 97%, at least 98%, at least 99% or 100%), more preferably 65%, even more preferably 70%, particularly preferable 75%, more particularly preferable 80% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the Napi2b antibody may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions listed in Table I, herein) is envisaged as long as the antibody retains its capability to specifically bind to Napi2b protein and/or its CDRs have an identity to the then substituted sequence (at least 60% ((e.g., at least 65%, at least 70%, at least 75%, at least 80%, at least 85%, at least 90%, at least 95%, at least 96%, at least 97%, at least 98%, at least 99% or 100%), more preferably 65%, even more preferably 70%, particularly preferable 75%, more particularly preferable 80% identical to the “original” CDR sequence).

Conservative substitutions are shown in Table I under the heading of “preferred substitutions”. If such substitutions result in a change in biological activity, then more substantial changes, denominated “exemplary substitutions” in Table I, or as further described below in reference to amino acid classes, may be introduced and the products screened for a desired characteristic.

### Chemical Modifications

The antibodies or antigen-binding variants or fragments thereof used in accordance with of the invention may be modified. Typical modifications conceivable in the context of the invention include, e.g., chemical modifications as described in the following.

Possible chemical modifications of the antibody or antigen-binding variants or fragments thereof include acylation or acetylation of the amino-terminal end or amidation or esterification of the carboxy-terminal end or, alternatively, on both. The modifications may also affect the amino group in the side chain of lysine or the hydroxyl group of threonine. Other suitable modifications include, e.g., extension of an amino group with polypeptide chains of varying length (e.g., XTEN technology or PASylation®), N-glycosylation, O-glycosylation, and chemical conjugation of carbohydrates, such as hydroxyethyl starch (e.g., HESylation®) or polysialic acid (e.g., PolyXen® technology). Chemical modifications such as alkylation (e. g., methylation, propylation, butylation), arylation, and etherification may be possible and are also envisaged.

### Antibody Drug Conjugate (ADC)

The therm antibody drug conjugate (or ADC) as used herein may refer to any antibody according to present invention conjugated to one or more drug moieties (e.g., cytotoxic payload). Preferably, the antibody drug conjugate (ADC) of the present invention comprising the anti-Napi2b antibody of the present invention (e.g., humanized monoclonal Napi2b-specific IgG1 antibody) conjugated to one or more cytotoxic payloads: (a) wherein the cytotoxic payload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or (b) wherein cytotoxic payload is a camptothecin moiety C selected from the group consisting of exatecan (e.g., CAS Nr: 171335-80-1), DXD, SN38, camptothecin, topotecan, irinotecan, belotecan, lurtotecan, rubitecan, silatecan, cositecan, and gimatecan; and/or (c) wherein the cytotoxic pyload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or (d) wherein the linker (L) comprises a valine-citrulline-PAB moiety or a valine-alanine-PAB moiety; and/or (e) wherein cytotoxic payload is Exatecan, conjugated via a chemical valine-citrulline-PAB or a valine-alanine-PAB release unit, wherein said release unit is cleavable by a protease. As used herein, a “linker” (L) may refer to any chemical moiety that is capable of linking the antibody of the present invention with one or more drug moieties (e.g., cytotoxic moieties (e.g., cytotoxic payloads, e.g., Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan); Preferably L is a phosphonamidate linker; further preferably linker L comprising a valine-citrulline-PAB moiety or a valine-alanine-PAB moiety; most preferably the linker L is cleavable (e.g., susceptible to enzymatic cleavage).

### Sequence Identity

The term “% identity” or “% sequence identity” as used herein may refer to the percentage of pair-wise identical residues—following (homologous) alignment of a sequence of a polypeptide of the invention with a sequence in question—with respect to the number of residues in the longer of these two sequences. Percent identity is determined by dividing the number of identical residues by the total number of residues and multiplying the product by 100.

The percentage of sequence homology or sequence identity can, for example, be determined herein using the BLASTP, version blastp 2.2.5 (November 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1) including the propeptide sequences, preferably using the wild type protein scaffold as reference in a pairwise comparison. It is calculated as the percentage of numbers of “positives” (homologous amino acids) indicated as result in the BLASTP program output divided by the total number of amino acids selected by the program for the alignment.

The term “Napi2b” refers to Sodium-dependent phosphate transport protein 2B and generally comprises all known isoforms. Preferably said Sodium-dependent phosphate transport protein 2B having SEQ ID NO: 1 or UniProtKB Accession Number: 095436.

### Vector

The nucleic acid of the invention may also be in the form of, may be present in and/or may be part of a vector.

The term “vector” refers a nucleic acid molecule used as a vehicle to transfer (foreign) genetic material into a host cell and encompasses—without limitation—plasmids, viruses, cosmids and artificial chromosomes such as bacterial artificial chromosomes (BACs) and yeast artificial chromosomes (YACs). In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Vectors may encompass additional elements besides the transgene insert and a backbone including gene regulation elements, genetic markers, antibiotic resistances, reporter genes, targeting sequences, or protein purification tags. Particularly envisaged within the context of the invention are expression vectors (expression constructs) for expression of the transgene in the host cell, which generally comprise—in addition to the transgene—gene regulation sequences.

An expression vector is, in general, a vector that can provide for expression of the antibodies of the present invention in vitro and/or in vivo (i.e. in a suitable host cell, host organism and/or expression system). The person skilled in the art will readily understand that choice of a particular vector include depends, e.g., on the host cell, the intended number of copies of the vector, whether transient or stable expression of the antibody of the present invention is envisaged, and so on.

“Transient expression” results from the introduction of a nucleic acid (e.g. a linear or non-linear DNA or RNA molecule) or vector that is incapable of autonomous replication into a recipient host cell. Expression of the transgene occurs through the transient expression of the introduced sequence.

However, “stable expression” of the nucleic acid sequence as described herein will often be preferred and may be accomplished by either stably integrating the nucleic acid sequence into the host cell's genome or by introducing a vector comprising the nucleic acid sequence of the invention and being capable of autonomously replicating into the host cell.

The vector provided herein is in particular envisaged to comprise a gene regulation element operably linked to the DNA sequence encoding antibody of the present invention.

The term “gene regulation element” refers to DNA sequences necessary for the expression of an operably linked coding sequence in a particular host organism. The term “gene regulation element” includes controllable transcriptional promoters, operators, enhancers, silencers, transcriptional terminators, 5′ and 3′ untranslated regions which interact with host cellular proteins to carry out transcription and translation and other elements that may control gene expression including initiation and termination codons. The precise nature of the regulatory regions needed for gene expression may vary from organism to organism. Prokaryotic gene regulation elements, for example, include a promoter, optionally an operator sequence, and a ribosome binding site (RBS), whereas gene regulation elements for eukaryotic cells comprise promoters, polyadenylation (poly-A) signals, and enhancers.

The gene regulation element is envisaged to be “operably linked” to the gene to be expressed, i.e. placed in functional relationship with the same. For instance, a promoter or enhancer is “operably linked” to a coding nucleic acid sequence if it affects the transcription of the sequence. The DNA sequences being “operably linked” may or may not be contiguous. Linking is typically accomplished by ligation at convenient restriction sites or synthetic oligonucleotide adaptors or linkers.

### Host Cell

Further provided herein is a host cell (e.g., recombinant and/or isolated host cell) comprising the vector as described herein.

A variety of host cells can be employed for expressing the nucleic acid sequence encoding antibodies as described herein. Host cells can be prepared using genetic engineering methods known in the art. The process of introducing the vector into a recipient host cell is also termed “transformation” or “transfection” hereinafter. The terms are used interchangeably herein.

Host cell transformation typically involves opening transient pores or “holes” in the cell wall and/or cell membrane to allow the uptake of material. Illustrative examples of transformation protocols involve the use of calcium phosphate, electroporation, cell squeezing, dendrimers, liposomes, cationic polymers such as DEAE-dextran or polyethylenimine, sonoporation, optical transfection, impalefection, nanoparticles (gene gun), magnetofection, particle bombardement, alkali cations (cesium, lithium), enzymatic digestion, agitation with glass beads, viral vectors, or others. The choice of method is generally dependent on the type of cell being transformed, the vector to be introduced into the cell and the conditions under which the transformation is taking place.

As used herein, the term “host cell” may refer to any cell or cell culture acting as recipients for the vector or isolated nucleic acid sequence encoding the Abs as described herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

E.g., the Abs can be produced in bacteria. In addition to prokaryotes, eukaryotic microbes such as filamentous fungi or yeast are suitable cloning or expression hosts for the NAPI2B-antibodies of the invention. Illustrative examples include Saccharomyces cerevisiae, Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antibody construct of the invention may also be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa califomica NPV and the Bm-5 strain of Bombyx mori NPV.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art.

Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO), mouse sertoli cells (TM4); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2,1413 8065); mouse mammary tumor (MMT 060562, ATCC CCL5 1); TRI cells; MRC 5 cells; FS4 cells; and human hepatoma cells (Hep G2).

### Patients

The term “patient” or “subject” as used herein refers to a human or non-human animal, generally a mammal. Particularly envisaged is a mammal, such as a rabbit, a mouse, a rat, a Guinea pig, a hamster, a dog, a cat, a pig, a cow, a goat, a sheep, a horse, a monkey, an ape or preferably a human. Thus, the methods, uses and compounds described in this document are in general applicable to both human and veterinary disease.

### Treatment

The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations of the diseases. The term “treatment” thus also includes the amelioration or prevention of cancer.

In the context with the present invention the term “therapeutic effect” in general refers to the desirable or beneficial impact of a treatment, e.g. amelioration or remission of the disease manifestations. The term “manifestation” of a disease is used herein to describe its perceptible expression, and includes both clinical manifestations, hereinafter defined as indications of the disease that may be detected during a physical examination and/or that are perceptible by the patient (i.e., symptoms), and pathological manifestations, meaning expressions of the disease on the cellular and molecular level. The therapeutic effect of treatment with the NaPi2b-ADC of the present invention can be assessed using routine methods in the art, e.g. measuring leukemia burden by blood/bone marrow analysis (cytomorphology, flow cytometry, genetcs), clinical chemistry or radiologic procedures (e.g. CT) Additionally or alternatively it is also possible to evaluate the general appearance of the respective patient (e.g., fitness, well-being) which will also aid the skilled practitioner to evaluate whether a therapeutic effect has been elicited. The skilled person is aware of numerous other ways which are suitable to observe a therapeutic effect of the compounds of the present invention.

### Dose

Preferably, a therapeutically effective amount of the compound as described herein is administered. By “therapeutically effective amount” is meant an amount of the compound as described herein that elicits a therapeutic effect. The exact dose of Ab-NaPi2b-ADC of the present invention will depend on the purpose of the treatment (e.g. remission induction, maintenance), and will be ascertainable by one skilled in the art using known techniques. Adjustments for route of administration, age, body weight, general health, sex, diet, time of administration, drug interaction and the severity of the condition may be necessary, and will be ascertainable with routine experimentation by those skilled in the art.

### Administration

A variety of routes are applicable for administration of the compound according to the present invention, including, but not limited to, orally, topically, transdermally, subcutaneously, intravenously, intraperitoneally, intramuscularly or intraocularly, preferably subcutaneously and/or intravenously. However, any other route may readily be chosen by the person skilled in the art if desired.

### Composition

It is envisaged to administer the NAPI2B antibodies and/or ADCs of the present invention in the form of a pharmaceutical composition.

The term “pharmaceutical composition” particularly refers to a composition suitable for administering to a human, i.e., a composition that is preferably sterile and/or contains components which are pharmaceutically acceptable. However, compositions suitable for administration to non-human animals are also envisaged herein. Preferably, a pharmaceutical composition comprises an Ab-NaPi2b-ADC of the present invention together with one or more pharmaceutical excipients. The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers or tonicity modifiers. Pharmaceutical compositions of the invention can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration.

The pharmaceutical composition of the present invention may further comprise one or more additional agents. Preferably, said agents are therapeutically effective for treatment the diseases described herein and present in the composition in a therapeutically effective amount.

In view of the above, the present invention hence also provides a pharmaceutical composition comprising one or more NaPi2b antibodies and/or ADCs of the present invention. Said pharmaceutical composition is particularly intended for use in a method of therapeutic and/or prophylactic treatment of cancer.

### Kit

A kit is also provided herein. The kit may be a kit of two or more parts, and comprises the NaPi2b antibodies and/or ADCs of the present invention, preferably in a therapeutically effective amount and in a pharmaceutically acceptable form. The components of the kit may be contained in a container or vials. The kit is envisaged to comprise additional agents useful in treating cancer.

In some aspects/embodiments the present invention relates to anti-NaPi2b antibody (e.g., an antibody against NPT2B_HUMAN Sodium-dependent phosphate transport protein 2B, e.g., having UniProt Accession Number: 095436 or SEQ ID NO: 1 and/or an antibody against NPT2B_RAT Sodium-dependent phosphate transport protein 2B, e.g., having UniProt Accession Number: Q9JJ09 or SEQ ID NO: 2), wherein said NaPi2b antibody is capable of the following: (a) binding to human Napi2b (e.g., SEQ ID NO: 1) and/or rat Napi2b (e.g., SEQ ID NO: 2), preferably said binding to said human Napi2b and said rat Napi2b having about the same K_{D}, further preferably said antibody is selected from the group consisting of: AV-25, AV-15, AV-18, AV-21 and AV-29 antibody), further most preferably said about same K_{D} having up to 50% difference (e.g., up to 45%, 40%, 35%, 30%, 25%, 20%, 15%, 10%, 9%, 8%, 7%, 6%, 7%, 5%, 4%, 3%, 2% or 1% difference); (b) cross-reactivity with rat Napi2b (e.g., having UniProt Accession Number: Q9JJ09 or SEQ ID NO: 2), (c) cross-reactivity with cynomolgus monkey (e.g., Macaca fascicularis) Napi2b (e.g., having UniProtKB Accession Number: A0A2K5UHY1 or SEQ ID NO: 3; (d) internalization, preferably by the means of the antigen-mediated antibody internalization; (e) optionally, not having a dipeptide deamidation site in a heavy chain variable region's (V_{H}) CDR2, preferably said absent dipeptide deamidation site is NG (Asn-Gly) in V_{H} CDR2.

In some aspects/embodiments the present invention relates to a monoclonal human or humanized IgG1 anti-NaPi2b antibody, preferably comprising kappa (κ) light chain.

In some aspects/embodiments the present invention relates to a hybridoma, wherein said hybridoma produces the antibody of the present invention.

In some aspects/embodiments the present invention relates to a nucleic acid encoding the antibody of the present invention.

In some aspects/embodiments the present invention relates to an expression vector comprising at least one of the nucleic acid molecules of the present invention.

The present invention further relates to In some aspects/embodiments the present invention relates to an isolated host cell (e.g., an isolated recombinant host cell) comprising the vector and/or nucleic acid of the present invention.

In some aspects/embodiments the present invention relates to an antibody drug conjugate (ADC) comprising the anti-NaPi2b antibody of the present invention.

In some aspects/embodiments the present invention relates to an antibody drug conjugate (ADC) of the present invention comprising the anti-NaPi2b antibody of the present invention (e.g., humanized monoclonal NaPi2b-specific IgG1 antibody) conjugated to a cytotoxic payload: (a) wherein the cytotoxic pyload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or (b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or (c) wherein the cytotoxic pyload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or (d) wherein the linker (L) comprises a valine-citrulline-PAB moiety or a valine-alanine-PAB moiety; and/or (e) wherein cytotoxic payload is Exatecan, conjugated via a chemical valine-citrulline-PAB or a valine-alanine-PAB release unit, wherein said release unit is cleavable by a protease.

In some aspects/embodiments the present invention relates to cancer. Cancer can be any cancer. Preferably said cancer is a solid and/or metastatic cancer, further preferably said cancer is selected from the group consisting of: lung cancer, ovarian cancer, thyroid cancer, nonsquamous non-small cell lung carcinoma, nonmucinous ovarian carcinoma, papillary thyroid carcinoma, renal cancer, endometrial cancer, uterus cancer, ureter cancer, bladder cancer and fallopian tube cancer.

In some aspects/embodiments the present invention relates to a composition or kit comprising the anti-NaPi2b, antibody drug conjugate (ADC), hybridoma, nucleic acid, expression vector and/or host cell of the present invention.

In some aspects/embodiments the present invention relates to method of synthesis of the antibody drug conjugates (ADCs) of the present invention.

In some aspects/embodiments the present invention relates to methods of treatment (e.g., of a patient) and uses of the antibody, antibody drug conjugate (ADC), nucleic acid, expression vector, host cell, composition and/or kit of the present invention.

In some aspects of the present invention, the antibody of the present invention is expressed as an Fc-silenced (LALA mutation) IgG1 in CHO cells, purified via Protein A chromatography.

### The Present Invention Further Relates to the Following Items

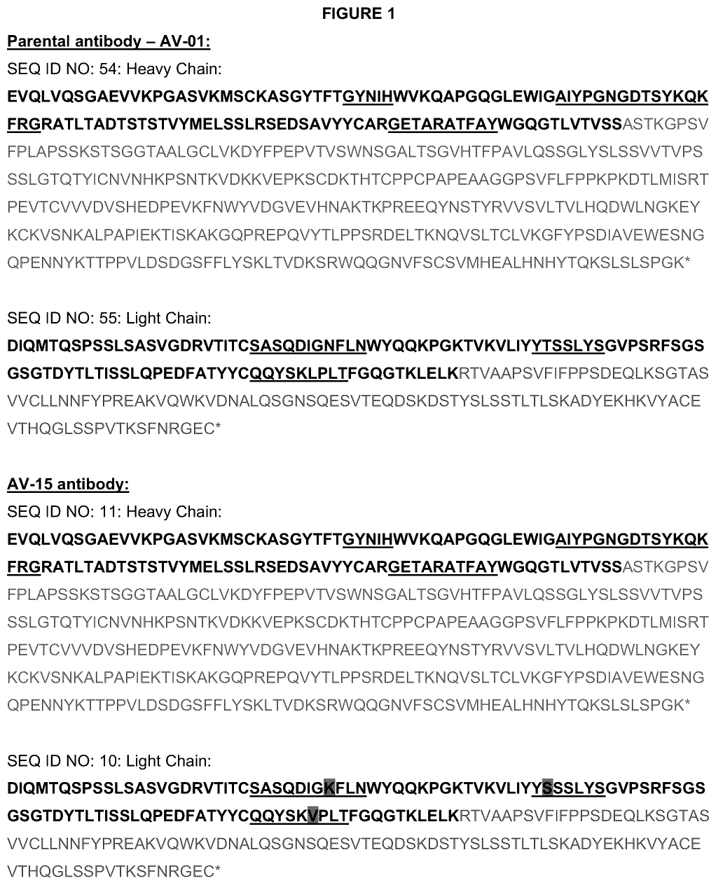

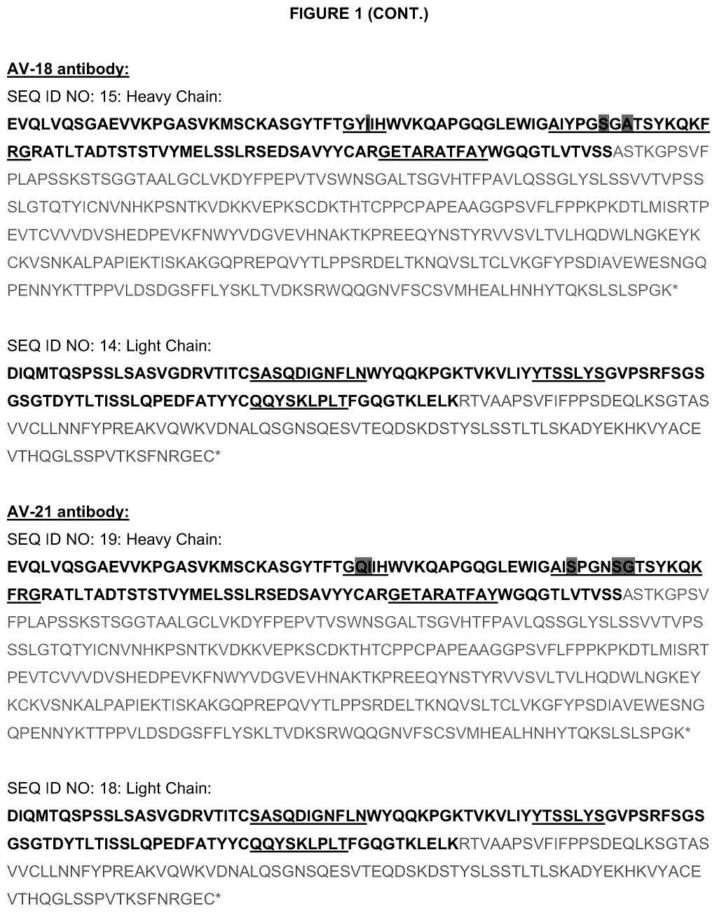

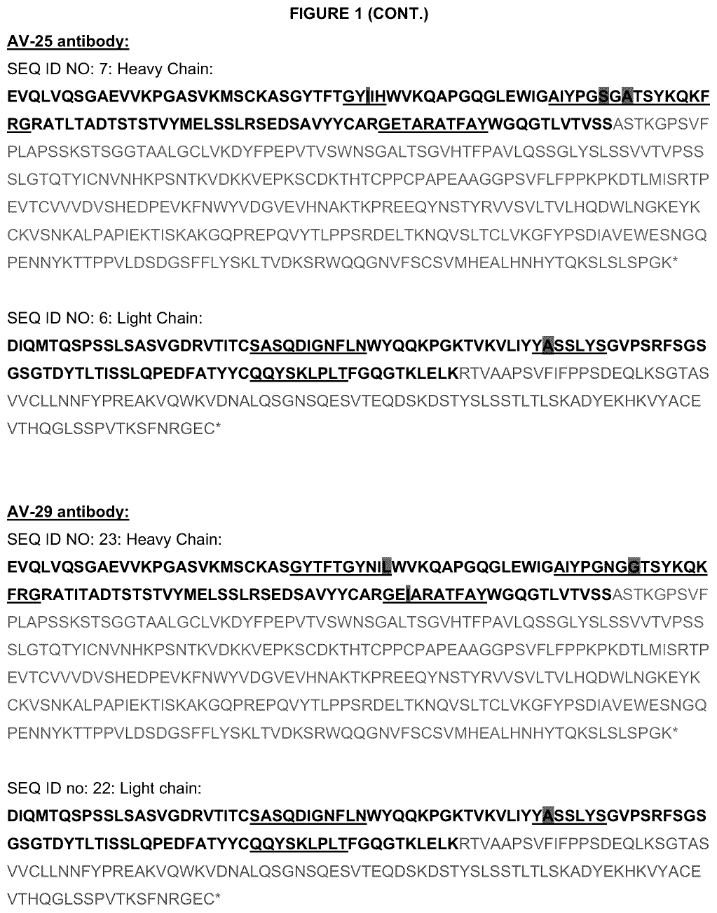

1. An anti-NaPi2b antibody (e.g., an antibody against NPT2B_HUMAN Sodium-dependent phosphate transport protein 2B, e.g., having UniProt Accession Number: 095436 or SEQ ID NO: 1 and/or an antibody against NPT2B_RAT Sodium-dependent phosphate transport protein 2B, e.g., having UniProt Accession Number: Q9JJ09 or SEQ ID NO: 2), wherein said NaPi2b antibody is capable of the following:
   a) binding to said human Napi2b (e.g., SEQ ID NO: 1) and/or rat Napi2b (e.g., SEQ ID NO: 2), preferably said binding to said human and rat Napi2b having about the same K_{D}, further preferably said antibody is selected from the group consisting of: AV-25, AV-15, AV-18, AV-21 and AV-29 antibody), further most preferably said about same K_{D} having up to 50% difference (e.g., up to 45%, 40%, 35%, 30%, 25%, 20%, 15%, 10%, 9%, 8%, 7%, 6%, 7%, 5%, 4%, 3%, 2% or 1% difference);
   b) cross-reactivity with rat Napi2b (e.g., having UniProt Accession Number: Q9JJ09 or SEQ ID NO: 2), preferably said cross-reactivity is improved (e.g., by at least 10%) compared to corresponding cross-reactivity with rat Napi2b (e.g., having UniProt Accession Number: Q9JJ09 or SEQ ID NO: 2) of the parental antibody (e.g., comprising SEQ ID NOs 54 and 55, e.g., as shown in FIG. 1), further preferably the cross-reactivity with rat Napi2b is measured with endogenous rat Napi2b, most preferably said endogenous Napi2b is located on the cell surface.
   c) cross-reactivity with cynomolgus monkey (e.g., Macaca fascicularis) Napi2b (e.g., having UniProtKB Accession Number: A0A2K5UHY1 or SEQ ID NO: 3;
   d) internalization, preferably by the means of the antigen-mediated antibody internalization; further preferably said internalization is improved (e.g., by at least 10%, e.g., 15%) compared to corresponding internalization of the parental antibody (e.g., comprising SEQ ID NOs 54 and 55, e.g., as shown in FIG. 1);
   e) optionally, not having a dipeptide deamidation site in a heavy chain variable region's (V_{H}) CDR2, preferably said absent dipeptide deamidation site is NG (Asn-Gly) in V_{H} CDR2.
2. The anti-NaPi2b antibody according to any one of the preceding items, wherein said anti-NaPi2b antibody not having a dipeptide deamidation site in a heavy chain variable region's (V_{H}) CDR2, preferably said absent dipeptide deamidation site is NG (Asn-Gly) in V_{H} CDR2, further preferably wherein the absence of said deamidation site decreases post-translational modification/s of corresponding V_{H} CDR2 (e.g., improving homogeneity and/or simplifying the production process of said antibody).
3. The anti-NaPi2b antibody according to any one of the preceding items, wherein said anti-NaPi2b antibody having cross-reactivity with rat Napi2b (e.g., having UniProt Accession Number: Q9JJ09 or SEQ ID NO: 2), preferably said cross-reactivity is improved (e.g., by at least 10%, e.g., 15%) compared to the corresponding cross-reactivity with rat Napi2b (e.g., having UniProt Accession Number: Q9JJ09 or SEQ ID NO: 2) of the parental antibody (e.g., comprising SEQ ID NOs 54 and 55, e.g., as shown in FIG. 1), further preferably wherein the cross-reactivity with rat Napi2b is measured with endogenous rat Napi2b, most preferably wherein said endogenous Napi2b is located on the cell surface.
4. The anti-NaPi2b antibody according to any one of the preceding items, wherein said anti-NaPi2b antibody is capable of internalization, preferably by the means of the antigen-mediated antibody internalization; further preferably said internalization is improved (e.g., by at least 10%, e.g., 15%) compared to corresponding internalization of the parental antibody (e.g., comprising SEQ ID NOs 54 and 55, e.g., as shown in FIG. 1).
5. The anti-NaPi2b antibody according to any one of the preceding items, wherein said anti-NaPi2b antibody is binding to said human Napi2b (e.g., SEQ ID NO: 1) and/or rat Napi2b (e.g., SEQ ID NO: 2) with an improved (e.g., by at least 10%, e.g., 15%) K_{D} compared to corresponding K_{D} of the parental antibody (e.g., comprising SEQ ID NOs 54 and 55, e.g., as shown in FIG. 1).
6. The anti-NaPi2b antibody according to any one of the preceding items, wherein said anti-NaPi2b antibody is capable of binding (e.g., specifically binding) to an extracellular domain of said NaPi2b (e.g., comprising amino acids 122-135 and/or amino acids 235-361 and/or 429-485 and/or amino acids 547-552 of the human Napi2b having SEQ ID NO: 1).
7. The anti-NaPi2b antibody according to any one of the preceding items, wherein said anti-NaPi2b antibody has one or more of the following characteristics:
   a) having KD to an endogenously expressed human Napi2b in the range from about 0.01 to about 10 nmol/L, preferably in the range from about 1 to about 10 nmol/L, further preferably from about 1 to about 7 nmol/L, further preferably from about 1 to about 4 nmol/L (e.g., in OVCAR-3 cells (e.g., HTB-161#, ATCC) endogenously expressing Napi2b), further preferably said KD is measured by the means of a FACS assay, further most preferably having said KD in the range from about 2.661 to about 6.644 nmol/L; and/or
   b) optionally, having KD in the range from about 0.01 to about 10 nmol/L to an immobilized exogenous full-length Napi2b and/or one or more fragments thereof, preferably said one or more fragments comprising at least one extracellular domain (ECD) of said Napi2b (e.g., said ECD comprising amino acids 122-135 and/or amino acids 235-361 and/or 429-485 and/or amino acids 547-552 of the human Napi2b having SEQ ID NO: 1) and/or one or more fragments of said ECD (e.g., having length from about 15 to about 30 amino acids), wherein said full-length Napi2b and/or one or more fragments thereof are fused or not fused to one or more protein tags (e.g., 6 x His-tags, FLAG, HA, V5, Fc-fusion, MBP, SUMO, TEV, GFP, TST), preferably said KD is measured by the means of an ELISA assay, preferably from about 0.05 to about 0.2 nmol/L, further preferably having said KD in the range from about 0.071 to about 0.147 nmol/L.
8. The anti-NaPi2b antibody according to any one of the preceding items, wherein said anti-NaPi2b antibody is an antibody comprising:
   a) a heavy chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 4 and a light chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 5; preferably said anti-NaPi2b antibody is the AV-25 antibody comprising: a light chain comprising SEQ ID NO: 6 and a heavy chain comprising SEQ ID NO: 7;
   b) a heavy chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 8 and a light chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 9; preferably said anti-NaPi2b antibody is the AV-15 antibody comprising: a light chain comprising SEQ ID NO: 10 and a heavy chain comprising SEQ ID NO: 11;
   c) a heavy chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 12 and a light chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 13; preferably said anti-NaPi2b antibody is the AV-18 antibody comprising: a light chain comprising SEQ ID NO: 14 and a heavy chain comprising SEQ ID NO: 15;
   d) a heavy chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 16 and a light chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 17; preferably said anti-NaPi2b antibody is the AV-21 antibody comprising: a light chain comprising comprising SEQ ID NO: 18 and a heavy chain comprising comprising SEQ ID NO: 19;
   e) a heavy chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 20 and a light chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 21; preferably said anti-NaPi2b antibody is the AV-29 antibody comprising: a light chain comprising comprising SEQ ID NO: 22 and and a heavy chain comprising comprising SEQ ID NO: 23.
9. The anti-NaPi2b antibody according to any one of the preceding items, wherein said anti-NaPi2b antibody is:
   a) an antibody comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26 and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29;
   b) an antibody comprising a heavy chain variable region comprising heavy chain CDR1 having the amino acid sequence as set forth in SEQ ID NO: 30, heavy chain CDR2 having the amino acid sequence as set forth in SEQ ID NO: 31, and heavy chain CDR3 having the amino acid sequence as set forth in SEQ ID NO: 32 and a light chain variable region comprising light chain CDR1 having the amino acid sequence as set forth in SEQ ID NO: 33, light chain CDR2 having the amino acid sequence as set forth in SEQ ID NO: 34, and light chain CDR3 having the amino acid sequence as set forth in SEQ ID NO: 35;
   c) an antibody comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38 and a light chain variable region comprising light chain CDR1 having the amino acid sequence as set forth in SEQ ID NO: 39, light chain CDR2 having the amino acid sequence as set forth in SEQ ID NO: 40, and light chain CDR3 having the amino acid sequence as set forth in SEQ ID NO: 41;
   d) an antibody comprising a heavy chain variable region comprising heavy chain CDR1 having the amino acid sequence as set forth in SEQ ID NO: 42, heavy chain CDR2 having the amino acid sequence as set forth in SEQ ID NO: 43, and heavy chain CDR3 having the amino acid sequence as set forth in SEQ ID NO: 44 and a light chain variable region comprising light chain CDR1 having the amino acid sequence as set forth in SEQ ID NO: 45, light chain CDR2 having the amino acid sequence as set forth in SEQ ID NO: 46, and light chain CDR3 having the amino acid sequence as set forth in SEQ ID NO: 47;
   e) an antibody comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50 and a light chain variable region comprising light chain CDR1 having the amino acid sequence as set forth in SEQ ID NO: 51, light chain CDR2 having the amino acid sequence as set forth in SEQ ID NO: 52, and light chain CDR3 having the amino acid sequence as set forth in SEQ ID NO: 53.
10. The anti-NaPi2b antibody according to any one of the preceding items, wherein said anti-NaPi2b antibody has one or more of the following characteristics:
   a) a monoclonal antibody;
   b) a chimeric antibody and/or humanized anbibody;
   c) specifically recognizes Napi2b overexpressed on cancer cells;
   d) human IgG antibody, preferably a human IgG1 antibody;
   e) comprising kappa (κ) light chain;
   f) comprising lambda (λ) light chain;
   g) comprising Fc silencing mutations such as leucine (L) to alanine (A) substitution at the position 234 and 235 (LALA mutations), wherein said LALA mutations are capable of reducing immunocell effector function/s;
   h) capable of being internalized by target cells (e.g., cancer cells) expressing Napi2b; preferably said internalized antibody is directed to lysosomes;
   i) a tumor-selective antibody, preferably said tumor is a liquid and/or solid tumor;
   j) a malignant-cell selective antibody;
   k) binding to said human and/or rat Napi2b in a glycosylation-dependent manner, wherein said antibody binds to a glycosylated form of said Napi2b protein;
   l) having K_{D} to an endogenously expressed human Napi2b in the range from about 0.01 to about 10 nmol/L, preferably in the range from about 1 to about 10 nmol/L, further preferably from about 1 to about 7 nmol/L, further preferably from about 1 to about 4 nmol/L (e.g., in OVCAR-3 cells (e.g., HTB-161#, ATCC) endogenously expressing Napi2b), further preferably said K_{D} is measured by the means of a FACS assay, further most preferably having said K_{D} in the range from about 2.661 to about 6.644 nmol/L;
   m) optionally, having K_{D} in the range from about 0.01 to about 10 nmol/L to an immobilized exogenous full-length Napi2b and/or one or more fragments thereof, preferably said one or more fragments comprising at least one extracellular domain (ECD) of said Napi2b (e.g., said ECD comprising amino acids 122-135 and/or amino acids 235-361 and/or 429-485 and/or amino acids 547-552 of the human Napi2b having SEQ ID NO: 1) and/or one or more fragments of said ECD (e.g., having length from about 15 to about 30 amino acids), wherein said full-length Napi2b and/or one or more fragments thereof are fused or not fused to one or more protein tags (e.g., 6 x His-tags, FLAG, HA, V5, Fc-fusion, MBP, SUMO, TEV, GFP, TST), preferably preferably said K_{D} is measured by the means of an ELISA assay, preferably from about 0.05 to about 0.2 nmol/L, further preferably having said K_{D} in the range from about 0.071 to about 0.147 nmol/L.
11. The anti-NaPi2b antibody according to any one of the preceding items, wherein said anti-NaPi2b antibody is a monoclonal antibody.
12. The anti-NaPi2b antibody according to any one of the preceding items, wherein said anti-NaPi2b antibody is a chimeric antibody and/or humanized anbibody.
13. The anti-NaPi2b antibody according to any one of the preceding items, wherein said anti-NaPi2b antibody is capable of specifically recognizes Napi2b overexpressed on cancer cells.
14. The anti-NaPi2b antibody according to any one of the preceding items, wherein said anti-NaPi2b antibody is a human IgG antibody, preferably a human IgG1 antibody.
15. The anti-NaPi2b antibody according to any one of the preceding items, wherein said anti-NaPi2b antibody comprising kappa (κ) light chain.
16. The anti-NaPi2b antibody according to any one of the preceding items, wherein said anti-NaPi2b antibody comprising lambda (λ) light chain.
17. The anti-NaPi2b antibody according to any one of the preceding items, wherein said anti-NaPi2b antibody comprising Fc silencing mutations such as leucine (L) to alanine (A) substitution at the position 234 and 235 (LALA mutations (e.g., in Fc region of said antibody)), wherein said LALA mutations are capable of reducing immunocell effector function/s, (e.g., wherein said antibody comprising a mutated (e.g., according to any one of the preceding items) Fc region (or fragment crystallizable region), e.g., the tail region of said antibody that when unmutated interacts with cell surface Fc receptors (e.g., wherein said Fc region of an immunoglobulin molecule composed of the constant regions of the heavy chains, e.g., and when unmutated is capable of binding to antibody receptors (Fc receptor) on cells and the Clq component of complement)).
18. The anti-NaPi2b antibody according to any one of the preceding items, wherein said anti-NaPi2b antibody capable of being internalized by target cells (e.g., cancer cells) expressing Napi2b; preferably said internalized antibody is directed to lysosomes.
19. The anti-NaPi2b antibody according to any one of the preceding items, wherein said anti-NaPi2b antibody is a tumor-selective antibody, preferably said tumor is a liquid and/or solid tumor.
20. The anti-NaPi2b antibody according to any one of the preceding items, wherein said anti-NaPi2b antibody is a malignant-cell selective antibody.
21. The anti-NaPi2b antibody according to any one of the preceding items, wherein said anti-NaPi2b antibody is capable of binding to said human and/or rat Napi2b in a glycosylation-dependent manner, wherein said antibody binds to a glycosylated form of said Napi2b protein.
22. The anti-NaPi2b antibody according to any one of the preceding items, wherein said anti-NaPi2b antibody having K_{D} to an endogenously expressed human Napi2b in the range from about 0.01 to about 10 nmol/L, preferably in the range from about 1 to about 10 nmol/L, further preferably from about 1 to about 7 nmol/L, further preferably from about 1 to about 4 nmol/L (e.g., in OVCAR-3 cells (e.g., HTB-161#, ATCC) endogenously expressing Napi2b), further preferably said K_{D} is measured by the means of a FACS assay, further most preferably having said K_{D} in the range from about 2.661 to about 6.644 nmol/L
23. The anti-NaPi2b antibody according to any one of the preceding items, wherein said anti-NaPi2b antibody having K_{D} in the range from about 0.01 to about 10 nmol/L to an immobilized exogenous full-length Napi2b and/or one or more fragments thereof, preferably said one or more fragments comprising at least one extracellular domain (ECD) of said Napi2b (e.g., said ECD comprising amino acids 122-135 and/or amino acids 235-361 and/or 429-485 and/or amino acids 547-552 of the human Napi2b having SEQ ID NO: 1) and/or one or more fragments of said ECD (e.g., having length from about 15 to about 30 amino acids), wherein said full-length Napi2b and/or one or more fragments thereof are fused or not fused to one or more protein tags (e.g., 6× His-tags, FLAG, HA, V5, Fc-fusion, MBP, SUMO, TEV, GFP, TST), preferably preferably said K_{D} is measured by the means of an ELISA assay, preferably from about 0.05 to about 0.2 nmol/L, further preferably having said K_{D} in the range from about 0.071 to about 0.147 nmol/L.
24. The anti-NaPi2b antibody according to any one of the preceding items, wherein said anti-NaPi2b antibody, wherein said antibody is coupled to a labelling group.
25. The anti-NaPi2b antibody according to any one of the preceding items, wherein said anti-NaPi2b antibody, wherein said antibody said antibody is obtainable by a hybridoma (e.g., said antibody is a recombinant antibody).
26. The anti-NaPi2b antibody according to any one of the preceding items, obtained according to Example 1 or 2 herein and/or having characteristics as described in Example 1 or 2 herein (e.g., FIGS. 1-33, particularly, 3, 4, 5, 6, 7, 8, 9, 19 and/or 20).
27. The anti-NaPi2b antibody according to any one of the preceding items, wherein the antibody comprises one or more (e.g., 2) CDRs, heavy chain variable regions, light chain variable regions, heavy chain, light chains and/or signal sequences according to any one of the preceding items, preferably selected from the group consisting of: SEQ ID NOs: 4-53.
28. The anti-NaPi2b antibody according to any one of the preceding items, wherein the antibody comprises at least one (e.g., 2) heavy and light chain, preferably according to any one of the preceding items.
29. The anti-NaPi2b antibody according to any one of the preceding items, comprising one or more (e.g., 2, 3, 4, 5, 6, 7, 8, 9 or 10) amino acid substitution/s (or mutations), preferably located in one or more regions selected from the group consisting of: CDRs (e.g., CDR1, CDR2, CDR3, e.g., CDR-H1, CDR-H2, CDR-H3, CDR-H1, CDR-L1, CDR-L2 and/or CDR-L3, e.g., according to any one of the preceding items, e.g., sequence listing as disclosed herein), V_{H} (variable region heavy chain), V_{L} (variable region light chain), C_{H} (constant region heavy chain) or C_{L} (constant region light chain), F(ab) and/or Fc region (e.g., as defined in FIG. 1 herein)
30. The anti-NaPi2b antibody according to any one of the preceding items, comprising one or more (e.g., 2, 3, 4, 5, 6, 7, 8, 9 or 10) highly conservative, conservative or equivalent amino acid substitution/s (or mutations), e.g., “Conservative or equivalent substitution/s” meaning substitutions as listed as “Exemplary Substitutions” in Table I below, “Highly conservative” substitutions as used herein meaning substitutions as shown under the heading “Preferred Substitutions” in Table I below:

TABLE I
Amino Acid Substitutions

| Original | Exemplary Substitutions | Preferred Substitutions |
|---|---|---|
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

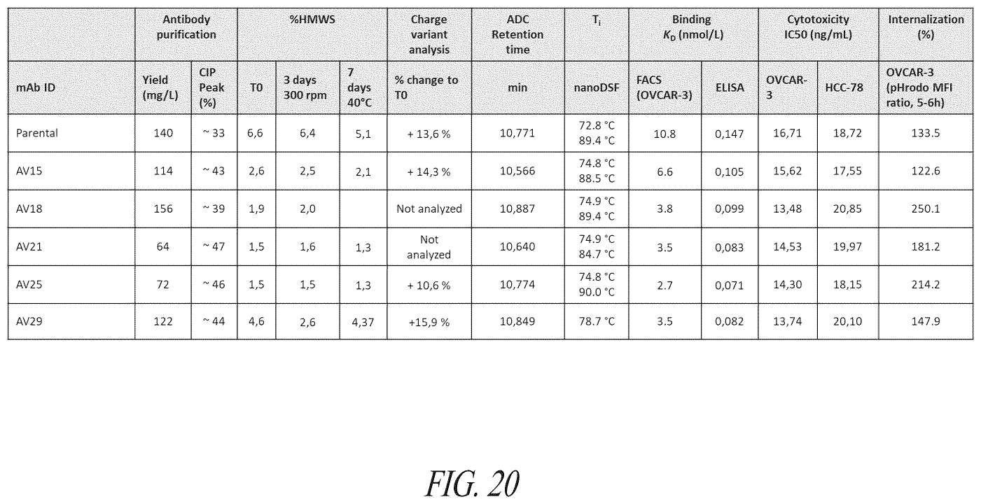

preferably said one or more (e.g., 2, 3, 4, 5, 6, 7, 8, 9 or 10) highly conservative, conservative or equivalent amino acid substitution/s (or mutations) are located in one or more regions selected from the group consisting of: CDRs (e.g., CDR1, CDR2, CDR3, e.g., CDR-H1, CDR-H2, CDR-H3, CDR-H1, CDR-L1, CDR-L2 and/or CDR-L3, e.g., according to any one of the preceding items, e.g., sequence listing as disclosed herein), V_{H} (variable region heavy chain), V_{L} (variable region light chain), C_{H} (constant region heavy chain) or C_{L} (constant region light chain), F(ab) and/or Fc region (e.g., as defined in FIG. 1 herein).
31. The antibody according to any one of the preceding items, wherein the % HMWS (high molecular weight species) of the antibody is less than 5% (e.g., as shown in FIG. 20).
32. A hybridoma, wherein said hybridoma produces the monoclonal antibody according to any one of the preceding items.
33. A nucleic acid encoding the antibody according to any one of the preceding items.
34. An expression vector comprising at least one of the nucleic acid molecules according to any one of the preceding items.
35. An isolated host cell (e.g., an isolated recombinant host cell) comprising the vector and/or nucleic acid according to any one of the preceding items.
36. An antibody drug conjugate (ADC) comprising the anti-NaPi2b antibody according to any one of the preceding items.
37. The antibody drug conjugate (ADC) according to any one of the preceding items, wherein said anti-NaPi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) cytotoxic moieties (e.g., cytotoxic payloads, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan), preferably via one or more linkers, further preferably via one or more phosphonamidate linkers.
38. The antibody drug conjugate (ADC) according to any one of the preceding items, wherein said anti-NaPi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan) cytotoxic moieties, preferably via one or more linkers, further preferably via one or more phosphonamidate linkers.
39. The antibody drug conjugate (ADC) according to any one of the preceding items, wherein said anti-NaPi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) Exatecan cytotoxic moieties via one or more linkers, preferably via one or more phosphonamidate linkers.
40. The antibody drug conjugate (ADC) according to any one of the preceding items, wherein said ADC comprising a humanized monoclonal NaPi2b-specific IgG1 antibody conjugated to a cytotoxic payload:
   a) wherein the cytotoxic pyload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or
   b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or
   c) wherein the cytotoxic pyload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or
   d) wherein the linker L comprises a valine-citrulline-PAB moiety or a valine-alanine-PAB moiety; and/or
   e) wherein cytotoxic payload is Exatecan, conjugated via a chemical valine-citrulline-PAB or a valine-alanine-PAB release unit, wherein said release unit is cleavable by a protease.
41. The antibody drug conjugate (ADC) according to any one of the preceding items, wherein said anti-NaPi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan) cytotoxic moieties via an ethynylphosphonamidate-linker/s conjugation (e.g., to all eight interchain-cysteine residues), preferably each phosphonamidate linker carries at least one PEG24 moiety (e.g., to prevent aggregation of the ADC), further preferably said ADC carries up to said eight linker payload moieties and eight PEG24 moieties.
42. The antibody drug conjugate (ADC) according to any one of the preceding items, wherein said ADC having a formula selected from the group consisting of:

### a) Formula I

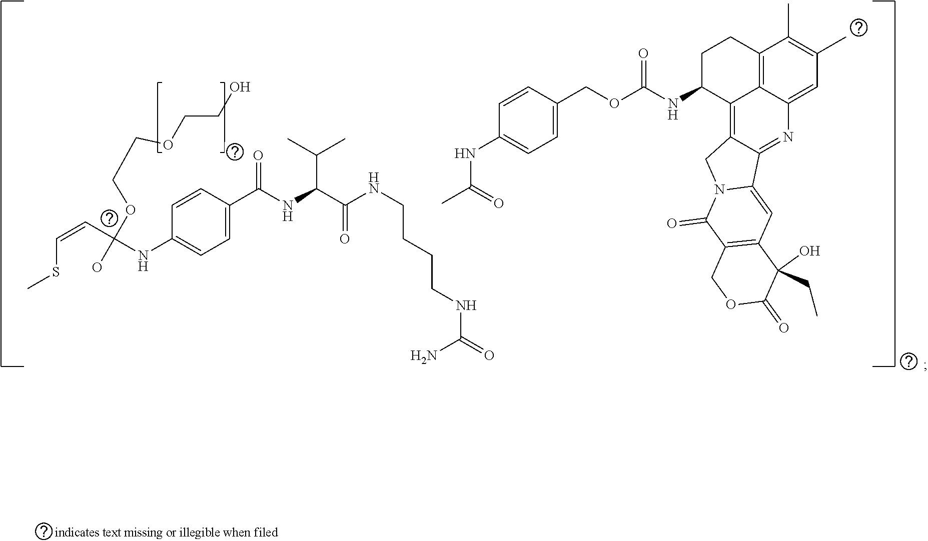

### b) Formula II

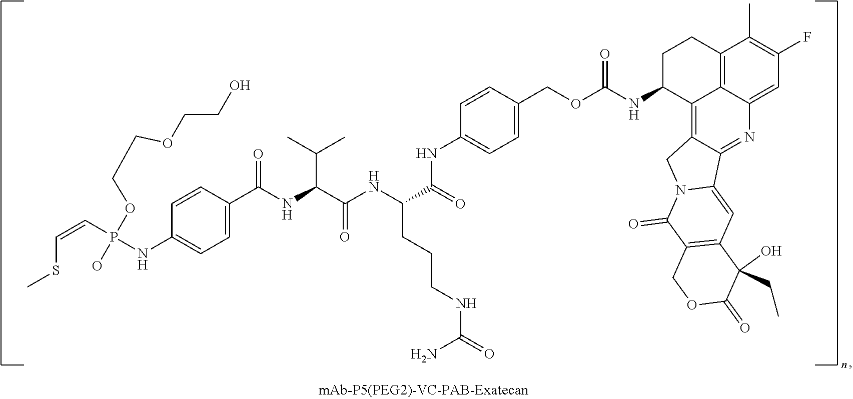 [0235] wherein n is in the range from 0 to 20 (e.g., 1, 2, 3, 4, 5, 6, 7 or 8);

### c) Formula III

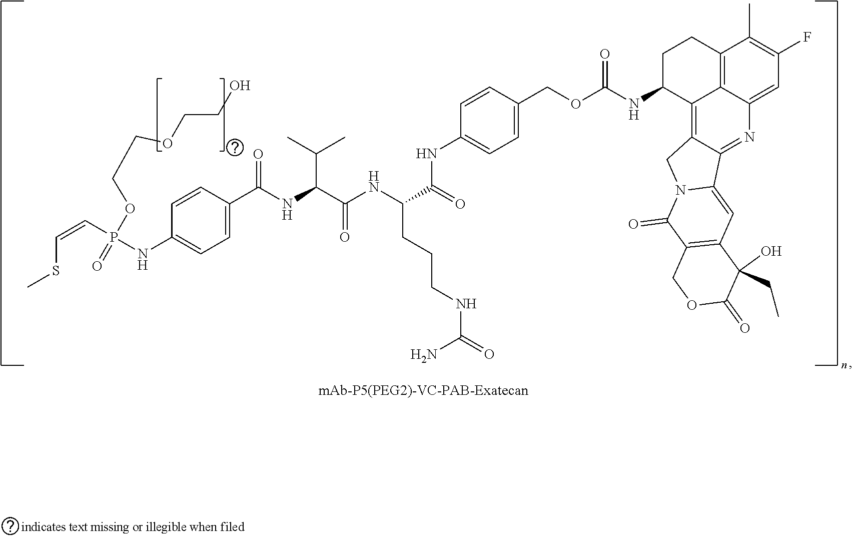 [0237] wherein n is in the range from 0 to 20 (e.g., 1, 2, 3, 4, 5, 6, 7 or 8);

### d) Formula IV

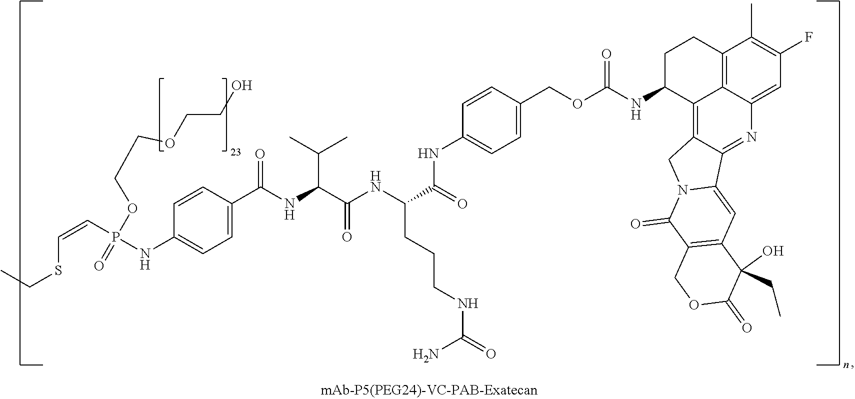 [0239] wherein n is in the range from 0 to 20 (e.g., 1, 2, 3, 4, 5, 6, 7 or 8);

### e) Formula V

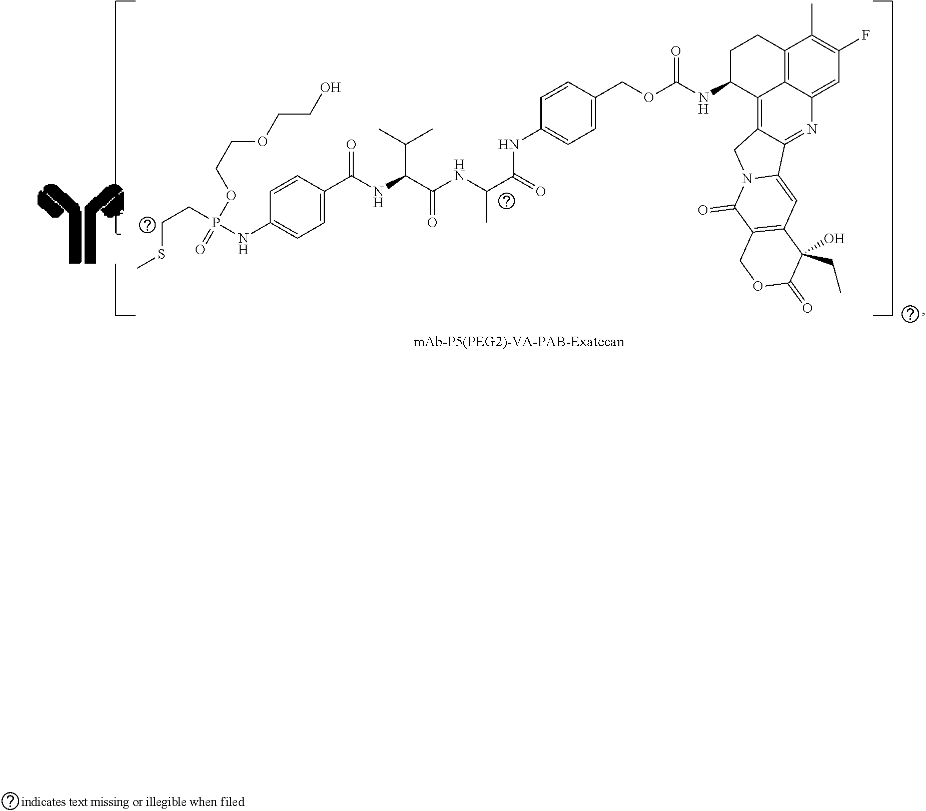 [0241] wherein n is in the range from 0 to 20 (e.g., 1, 2, 3, 4, 5, 6, 7 or 8);

### f) Formula VI

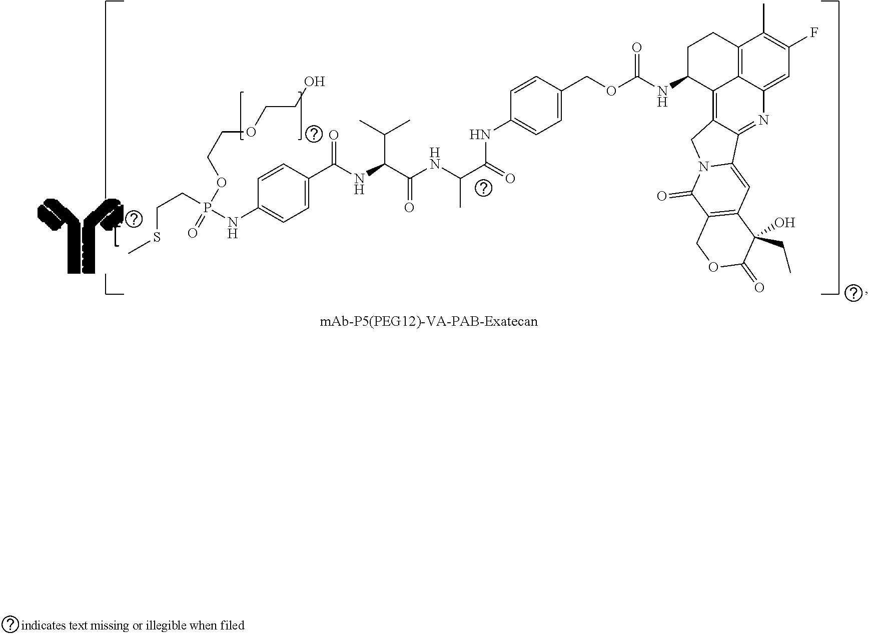 [0243] wherein n is in the range from 0 to 20 (e.g., 1, 2, 3, 4, 5, 6, 7 or 8);

### g) Formula VII

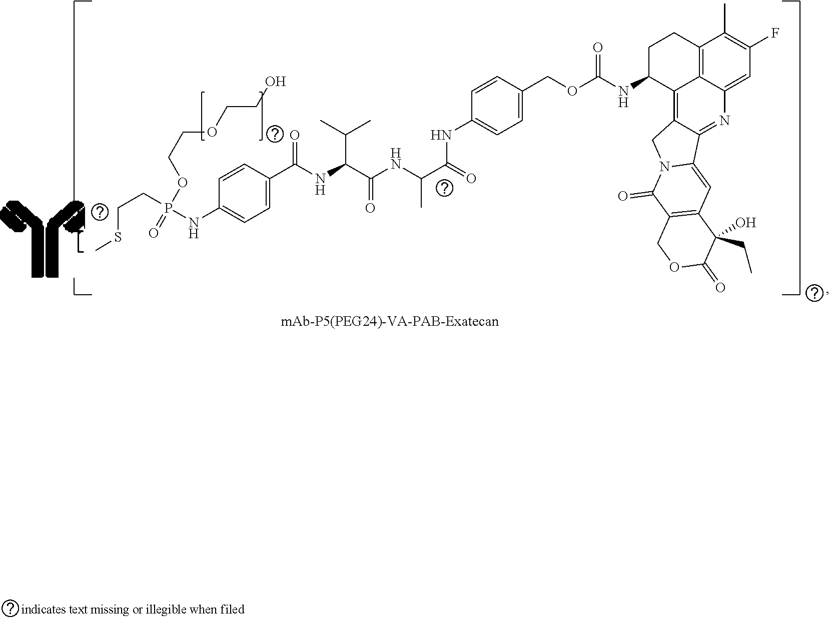 [0245] wherein n is in the range from 0 to 20 (e.g., 1, 2, 3, 4, 5, 6, 7 or 8);

### h) Formula VIII

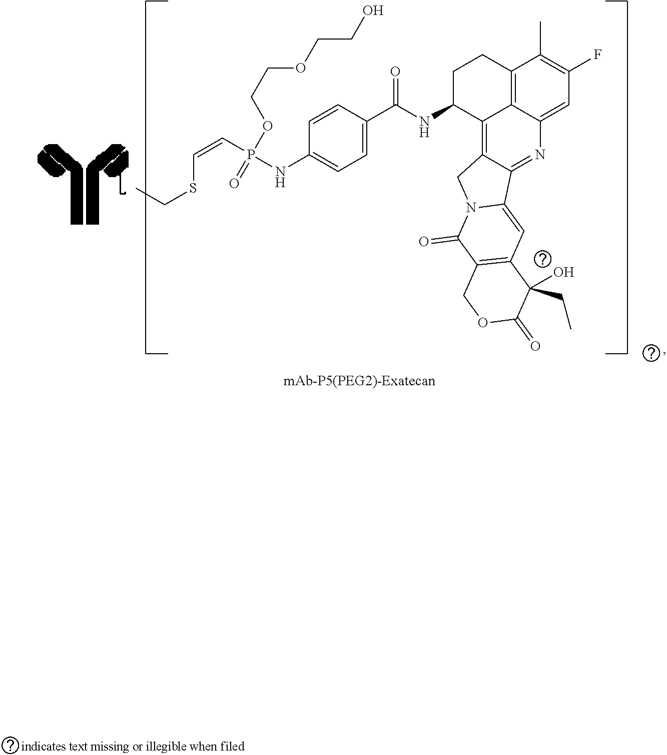 [0247] wherein n is in the range from 0 to 20 (e.g., 1,2,3,4,5,6,70r8);

### i) Formula IX

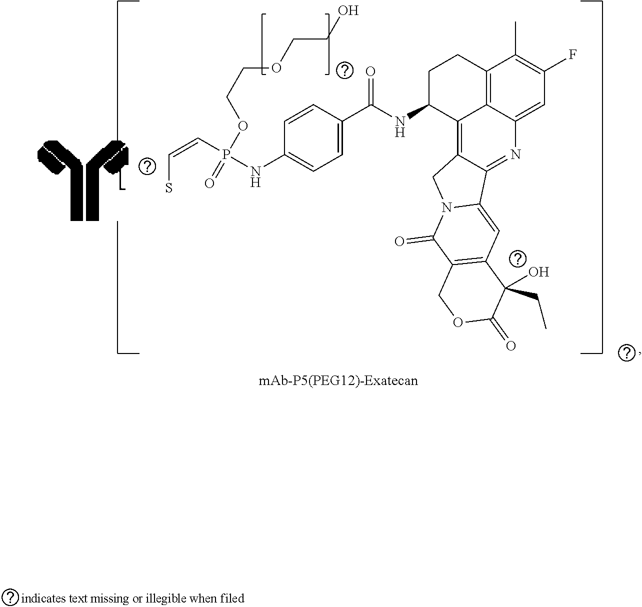 [0249] wherein n is in the range from 0 to 20 (e.g., 1,2,3,4,5,6,70r8);

### j) Formula X

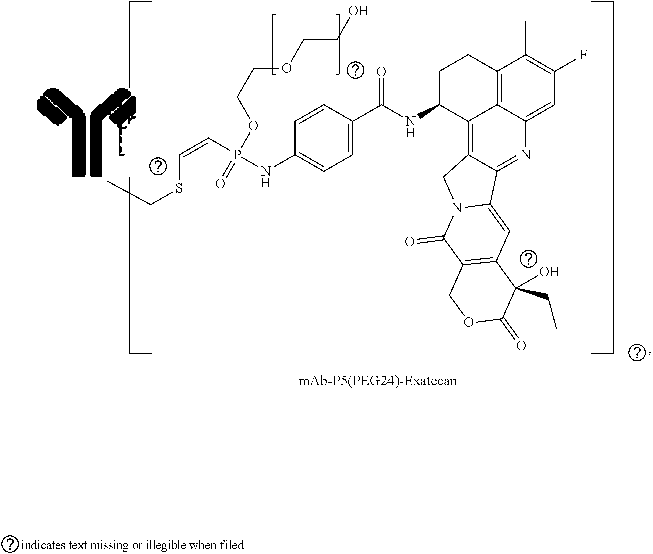 [0251] wherein n is in the range from 0 to 20 (e.g., 1,2,3,4,5,6,70r8);

### k) Formula XI

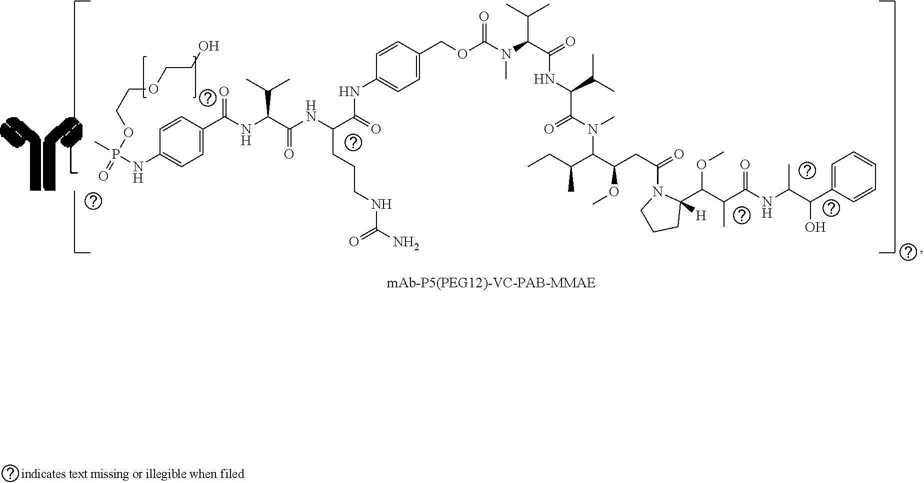 [0253] wherein n is in the range from 0 to 20 (e.g., 1, 2, 3, 4, 5, 6, 7 or 8);

### l) Formula XII

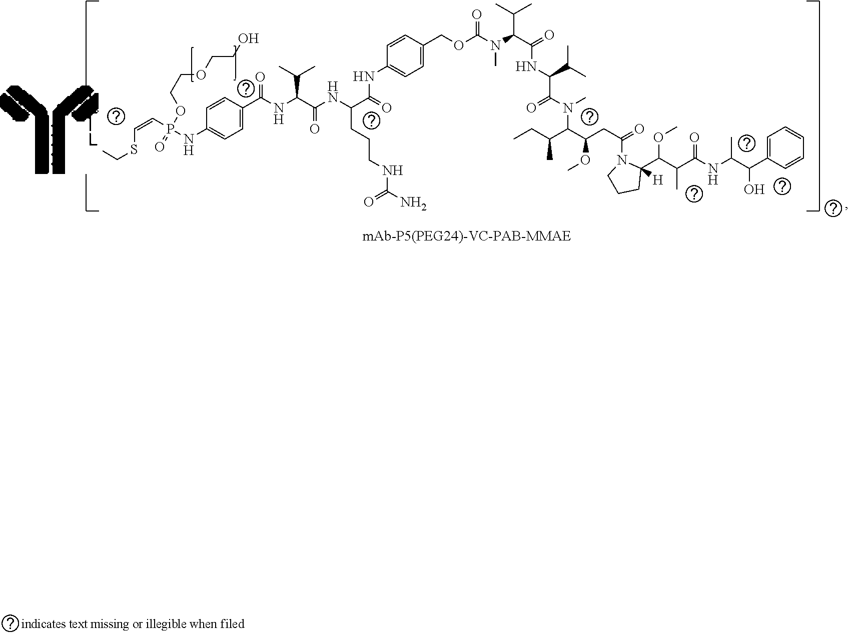 [0255] wherein n is in the range from 0 to 20 (e.g., 1, 2, 3, 4, 5, 6, 7 or 8);

### m) Formula XIII

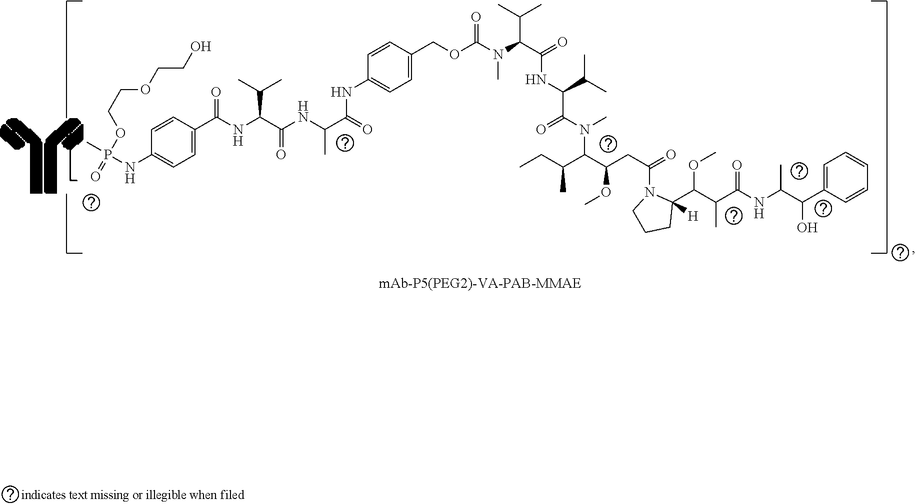 [0257] wherein n is in the range from 0 to 20 (e.g., 1, 2, 3, 4, 5, 6, 7 or 8);

### n) Formula XIV

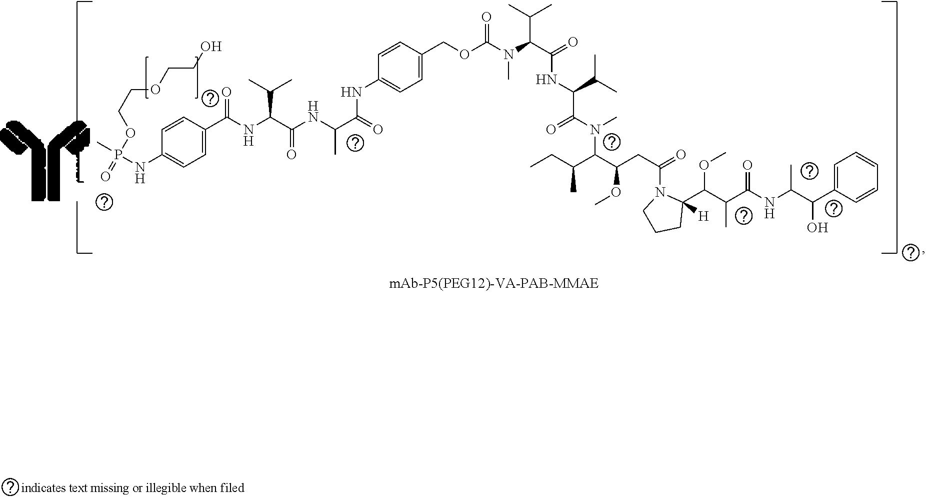 [0259] wherein n is in the range from 0 to 20 (e.g., 1, 2, 3, 4, 5, 6, 7 or 8);

### o) Formula XV

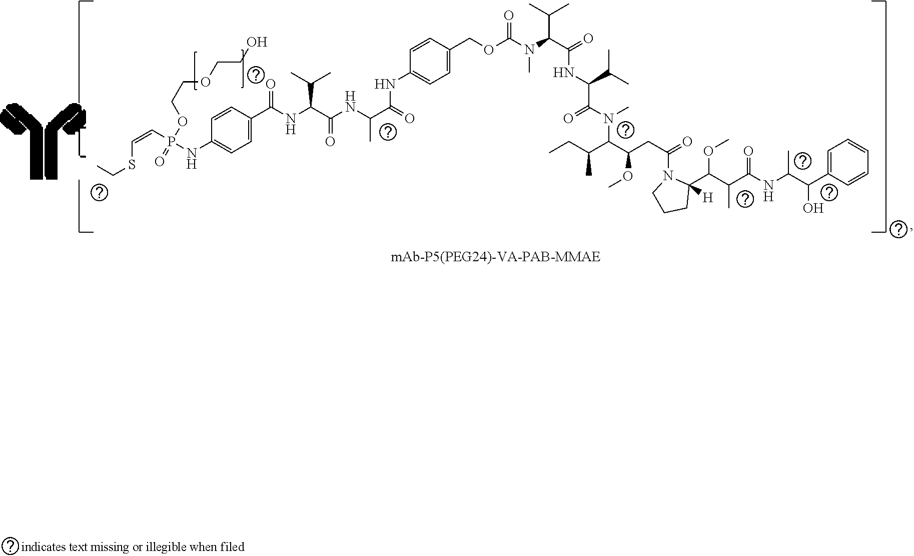 [0261] wherein n is in the range from 0 to 20 (e.g., 1, 2, 3, 4, 5, 6, 7 or 8);

### p) Formula XVI

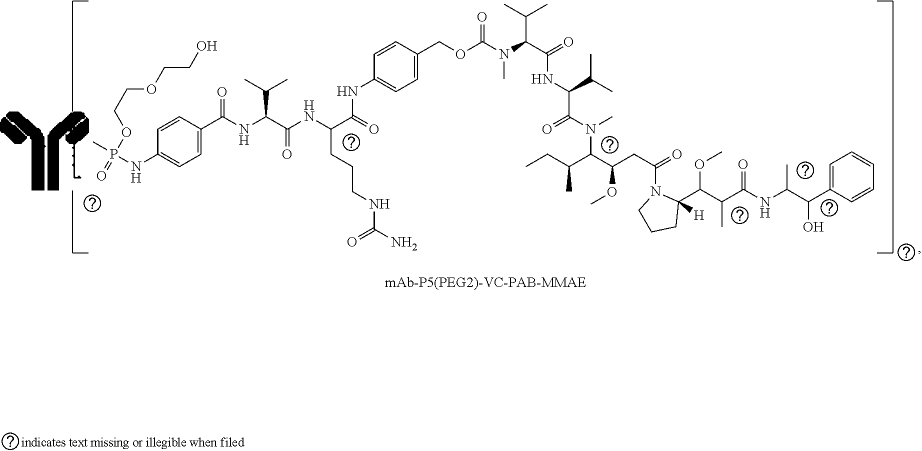 [0263] wherein n is in the range from 0 to 20 (e.g., 1, 2, 3, 4, 5, 6, 7 or 8).
43. The antibody drug conjugate (ADC) according to any one of the preceding items, wherein:
   a) said anti-NaPi2b monoclonal antibody is capable of specifically recognizing human Napi2b (e.g., SEQ ID NO: 1) and/or rat Napi2b (e.g., SEQ ID NO: 2) overexpressed on cancer cell/s; and
   b) upon binding of said ADC to said Napi2b overexpressed on cancer cells said ADC is capable of being internalized by the cells and trafficked into the lysosomal compartment, in which preferably a lysosomal protease (e.g., Cathepsin B) is capable of releasing said cytotoxic payload from the said ADC.
44. The antibody drug conjugate (ADC) according to any one of the preceding items, wherein the % HMWS (high molecular weight species) of the antibody is less than 5% (e.g., as shown in FIG. 20).
45. A method of producing an antibody drug conjugate (ADC), said method comprising:
   (a) conjugating the antibody according to any one of the preceding items to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) cytotoxic moieties (e.g., cytotoxic payloads, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan (e.g., CAS Nr: 171335-80-1), preferably via one or more linkers, further preferably via one or more phosphonamidate linkers.
46. The method of producing an antibody drug conjugate (ADC) according to any one of the preceding items, wherein said anti-Napi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan (e.g., CAS Nr: 171335-80-1)) cytotoxic moieties, preferably via one or more linkers, further preferably via one or more phosphonamidate linkers.
47. The method of producing an antibody drug conjugate (ADC) according to any one of the preceding items, wherein said anti-Napi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) Exatecan cytotoxic moieties via one or more linkers, preferably via one or more phosphonamidate linkers.
48. The method of producing an antibody drug conjugate (ADC) according to any one of the preceding items, wherein said anti-Napi2b antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan (e.g., CAS Nr: 171335-80-1)) cytotoxic moieties via an ethynylphosphonamidate-linker/s conjugation (e.g., to all eight interchain-cysteine residues), preferably each phosphonamidate linker carries at least one PEG moiety, with up to 24 PEG units y (e.g., to prevent aggregation of the ADC), further preferably said ADC carries up to said eight linker payload moieties and eight PEG24 moieties.
49. The method of producing an antibody drug conjugate (ADC) according to any one of the preceding items, wherein said ADC comprising a humanized monoclonal Napi2b-specific IgG1 antibody conjugated to a cytotoxic payload:
   a) wherein the cytotoxic pyload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or
   b) wherein cytotoxic payload is a camptothecin moiety C selected from the group consisting of exatecan (e.g., CAS Nr: 171335-80-1), DXD, SN38, camptothecin, topotecan, irinotecan, belotecan, lurtotecan, rubitecan, silatecan, cositecan, and gimatecan; and/or
   c) wherein the cytotoxic pyload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or
   d) wherein the linker L comprises a valine-citrulline-PAB moiety or a valine-alanine-PAB moiety; and/or
   e) wherein cytotoxic payload is Exatecan (e.g., CAS Nr: 171335-80-1), conjugated via a chemical valine-citrulline-PAB or a valine-alanine-PAB release unit, wherein said release unit is cleavable by a protease.
50. An antibody drug conjugate (ADC) produced by the method according to any one of the preceding items.
51. The antibody drug conjugate (ADC) according to any one of the preceding items, wherein the % HMWS (high molecular weight species) of the antibody is less than 5%, preferably thus decreasing aggregation of said ADC and/or decreasing toxicity of said ADC against the normal (e.g., non-cancer) tissue.
52. The antibody drug conjugate (ADC) according to any one of the preceding items, wherein the drug to antibody ratio (DAR) is in the range between 0 and 20, preferably is in the range between 1 and 20, further preferably is in the range between 2 and 12, most preferably is in the range between 4 and 10, further most preferably is in the range between 4 and 8.
53. The antibody drug conjugate (ADC) according to any one of the preceding items, wherein DAR of said ADC is 4 or 8, preferably 8.
54. A composition or kit comprising said anti-Napi2b, antibody drug conjugate (ADC), hybridoma, nucleic acid, expression vector and/or host cell according to any one of the preceding items.
55. The composition according to any one of the preceding items, wherein said composition is a pharmaceutical and/or diagnostic composition.
56. The composition or kit according to any one of preceding items, wherein a drug (e.g., cytotoxic moieties (e.g., cytotoxic payloads, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan) to antibody ratio (DAR) is in the range between 0 and 20, preferably is in the range between 1 and 20, further preferably is in the range between 2 and 12, most preferably is in the range between 4 and 10, further most preferably is in the range between 4 and 8.
57. A composition or kit comprising said anti-NaPi2b antibody, antibody drug conjugate (ADC), hybridoma, nucleic acid, expression vector and/or host cell according to any one of the preceding items.
58. A method for treatment, amelioration, prophylaxis and/or diagnostics of cancer, preferably said cancer is a solid and/or metastatic cancer, further preferably said cancer is selected from the group consisting of: lung cancer, ovarian cancer, thyroid cancer, nonsquamous non-small cell lung carcinoma, nonmucinous ovarian carcinoma, papillary thyroid carcinoma, renal cancer, endometrial cancer, uterus cancer, ureter cancer, bladder cancer and fallopian tube cancer, said method comprising: administering a therapeutically or prophylactically effective amount of the antibody, antibody drug conjugate (ADC), nucleic acid, expression vector, host cell, composition or kit according to any one of the preceding items.
59. The antibody, antibody drug conjugate (ADC), nucleic acid, expression vector, host cell, composition or kit according to any one of the preceding items, for use as a medicament and/or in therapy.
60. The antibody, antibody drug conjugate (ADC), nucleic acid, expression vector, host cell, composition or kit according to any one of the preceding items, for use in one or more of the following methods:
   (a) method for treatment, amelioration, prophylaxis and/or diagnostics of cancer, preferably said cancer is a solid and/or metastatic cancer, further preferably said cancer is selected from the group consisting of: lung cancer, ovarian cancer, thyroid cancer, nonsquamous non-small cell lung carcinoma, nonmucinous ovarian carcinoma, papillary thyroid carcinoma, renal cancer, endometrial cancer, uterus cancer, ureter cancer, bladder cancer and fallopian tube cancer;
   (b) method for monitoring development of cancer and/or for assessing the efficacy of cancer therapy;
   (c) method for screening a candidate compound for anti-cancer activity;
   (d) method for altering resistance of cancer cells to chemotherapy;
   (e) method for sensitizing cancer cells to chemotherapy;
   (f) method for inhibiting the growth of cancer cell expressing NaPi2b;
   (g) method for production or preparation of an antibody;
   (h) method for immunizing a non-human animal;
   (i) method for preparation of a hybridoma;
   (j) method according to any one of the preceding items;
   (k) method according to any one of (a)-(j), wherein said method is an in vivo, in vitro, or ex vivo method.
61. Use of the antibody, antibody drug conjugate (ADC), nucleic acid, expression vector, host cell, composition or kit according to any one of the preceding items, for one or more of the following:
   (a) for treatment, amelioration, prophylaxis and/or diagnostics of cancer, preferably said cancer is a solid and/or metastatic cancer, further preferably said cancer is selected from the group consisting of: lung cancer, ovarian cancer, thyroid cancer, nonsquamous non-small cell lung carcinoma, nonmucinous ovarian carcinoma, papillary thyroid carcinoma, renal cancer, endometrial cancer, uterus cancer, ureter cancer, bladder cancer and fallopian tube cancer;
   (b) for monitoring development of cancer and/or for assessing the efficacy of cancer therapy;
   (c) for screening a candidate compound for anti-cancer activity;
   (d) for altering resistance of cancer cells to chemotherapy;
   (e) for sensitizing cancer cells to chemotherapy;
   (f) for inhibiting the growth of cancer cell expressing NaPi2b;
   (g) for production or preparation of an antibody;
   (h) for immunizing a non-human animal;
   (i) for preparation of a hybridoma;
   (j) in a method according to any one of the preceding items;
   (k) use according to any one of (a)-(j), wherein said use is an in vivo, in vitro, or ex vivo use.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

The term “less than” or in turn “more than” does not include the concrete number.

For example, less than 20 means less than the number indicated. Similarly, more than or greater than means more than or greater than the indicated number, e.g., more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES OF THE INVENTION

An even better understanding of the present invention and of its advantages will be evident from the following examples, offered for illustrative purposes only. The examples are not intended to limit the scope of the present invention in any way.

### Example 1

### General Information

### Chemicals, Solvents and Antibodies

Chemicals and solvents were purchased from Merck (Merck group, Germany), TCI (Tokyo chemical industry CO., LTD., Japan), Iris Biotech (Iris Biotech GmbH, Germany), MCE (MedChemExpress, USA) and Carl Roth (Carl Roth GmbH+Co. KG, Germany) and used without further purification. Dry solvents were purchased from Merck (Merck group, Germany). PEG24 was purchased from BiochemPEG (Pure Chemistry Scientific Inc., United States).

### Preparative HPLC

Preparative HPLC was performed on a BUCHI Pure C-850 Flash-Prep system (BUCHI Labortechnik AG, Switzerland) using a VP 250/10 Macherey-Nagel Nucleodur C18 HTec Spum column (Macherey-Nagel GmbH & Co. Kg, Germany) for smaller scales. The following gradients were used: Method C: (A=H2O+0.1% TFA (trifluoroacetic acid), B=MeCN (acetonitrile)+0.1% TFA, flow rate 6 ml/min, 30% B 0-5 min, 30-70% B 5-35 min, 99% B 35-45 min. For bigger scales, a VP 250/21 Macherey-Nagel Nucleodur C18 HTec Spum column (Macherey-Nagel GmbH & Co. Kg, Germany) was used with the following gradients were used: Method D: (A=H2O+0.1% TFA (trifluoroacetic acid), B=MeCN (acetonitrile)+0.1% TFA, flow rate 14 ml/min, 30% B 0-5 min, 30-70% B 5-35 min, 99% B 35-45 min.

### LC-MS

Small molecules, linker-payloads, antibodies and ADCs were analyzed using a Waters H-class instrument equipped with a quaternary solvent manager, a Waters sample manager-FTN, a Waters PDA detector and a Waters column manager with an Acquity UPLC protein BEH C4 column (300 Å, 1.7 μm, 2.1 mm×50 mm) for antibodies and ADCs. Here, samples were eluted at a column temperature of 80° C. The following gradient was used: A: 0.1% formic acid in H₂O; B: 0.1% formic acid in MeCN. 25% B 0-1 min, 0.4 mL/min, 25-95% B 1-3.5 min 0.2 mL/min, 95% B 3.5-4.5 min 0.2 mL/min, 95-25% B 4.5-5 min 0.4 mL/min, 25-95% B 5-5.5 min 0.4 mL/min, 95-25% B 5.5-7.5 min 0.4 mL/min. Mass analysis was conducted with a Waters XEVO G2-XS QT of analyzer. Proteins were ionized in positive ion mode applying a cone voltage of 40 kV. Raw data was analyzed with MaxEnt 1. Small molecules and linker-payloads were analyzed with an Acquity UPLC-BEH C18 column (300 Å, 1.7 μm,2.1 mm×50 mm). Here, samples were eluted at a column temperature of 45° C. with a flow rate of 0.4 mL/min. The following gradient was used: A: 0.1% formic acid in H₂O; B: 0.1% formic acid in MeCN. 2% B 0-1 min, 2-98% B 1-5 min, 98%B 5-5.5 min, 98-2% B 5.5-6 min, 2% B 6-7min.

### CDR Mutagenisis and Antibody Expression

For the CDR mutagenesis campaign of the parental abtibody, a phage display library of 22×10⁸ sequences (66% functional) was prepared based on homology modeling of antibody Fv regions, selection of surface CDR residues possibly involved in antigen binding (VH: 18 positions , VK: 16 positions identified) and analysis of amino acid usage at each position in NGS data (approx. 2×10⁶ IgG sequences) (FIG. 1). Panning of the CDR mutation phage library for selection of affinity improved antibodies was carried out on antigen/Napi2b-expressing cells in 4 rounds of sequential antigen restriction achieved by increasing cell dilution. From the panning, 384 clones from each round were chosen (in total 1536 clones), produced as soluble scFv antibodies in E. coli, and screened for binding to human-Napi2b-expressing HEK293 cells and parental HEK293 cells using flow cytometry analysis. Signal to noise (N/S) between MFI of positive and MFI of negative cell line greater than 12 was chosen as hit selection criterium. According to the described selection criterium, 375 clones were identified and DNA-sequenced. From these results, 255 unique antibody sequences were identified and the off-rate of these clones was further analysed using BLI technology. In detail, biotinylated Napi2b-antigen was immobilized onto Streptavidin sensors and scFv association and dissociation was measured. The off-rate kinetics were modelled and clones fulfilling the following criteria, a significant binding response and high modelling accuracy had to be achieved, were ranked. 5 antibody clones/sequences were selected for further in-depth analysis: AV-15, AV-18, AV-21, AV-25 and AV-29 (FIG. 1).

Antibodies were then transiently expressed in Expi-CHO-S cells (Thermo Fisher) by co-transfecting cells with pcDNA3.4 expression plasmids (Thermo Fisher), coding for the heavy and light chain of the respective sequences in a 1:1 ratio, using the Expi-CHO transfection system (Thermo Fisher). Cells were harvested by centrifugation at 300 g for 5 minutes at 4° C. To clear micro particles from supernatant, supernatants were centrifuged at 4000-5000 g for 30 min at 4° C. For further clarification supernatants were passed through a 0.22 μm filter. Antibodies were purified from cleared and filtered supernatants via Protein A chromatography and analyzed by HPLC-SEC, HPLC-HIC, LC-MS and SDS-PAGE.

### Preparative Size-Exclusion-Chromatography

Protein purification by size-exclusion chromatography was conducted with an AKTA Pure FPLC system (GE Healthcare, United States) equipped with a F9-C-fraction collector.

### ADC Concentration Determination

The ADC concentrations were determined in a 96-well plate with a Pierce™ Rapid Gold BCA Protein Assay Kit (Thermo Fisher Scientific, USA) and a Bradford reagent B6916 (Merck, Germany) with pre-diluted protein assay standards of bovine gamma globulin (Thermo Fisher Scientific, USA). Results of both Assays were arithmetically averaged.

### Sample Preparation of ADCs and Antibodies for MS

For antibody and ADC deglycosylation, 0.5 μl PNGase-F solution (Pomega, Germany, Recombinant, cloned from Elizabethkingia miricola 10 u/μl) and 5 μL of a 100 mM solution of DTT in water were added to 50 μl of 0.2 mg/mL antibody or ADC in PBS and the solution was incubated at 37° C. for at least 2 hours. Glycosylated mAbs and ADCs were incubated at a concentration of 0.2 mg/ml with 10 mM DTT for 1 hour. Samples were subjected to LC/MS, injecting 2 μl for each sample.

### Analytical Size-Exclusion Chromatography

Analytical size-exclusion chromatography (A-SEC) of the ADCs was conducted on a Vanquish Flex UHPLC System with a DAD detector, Split Sampler FT (4° C.), Column Compartment H (25° C.) and binary pump F (Thermo Fisher Scientific, USA) using a MAbPac SEC-1 300Å, 4×300 mm column (Thermo Fisher Scientific, USA) with a flow rate of 0.15 mL/min. Separation of different ADC/mAb populations have been achieved during a 30 minute isocratic gradient using a phosphate buffer at pH 7 (20 mM Na2HPO4/NaH2PO4, 300 mM NaCl, 5% v/v isopropyl alcohol as a mobile phase. 8 μg ADC/mAb where loaded onto the column for A-SEC analysis. UV chromatograms were recorded at 220 and 280 nm.

### Analytical Hydrophobic Interaction Chromatography

The measurements were conducted on a Vanquish Flex UHPLC System (2.9) with a MabPac HIC Butyl 4.6×100 mm column (Thermo Fischer Scientific, USA). Separation of different ADCs/antibodies have been achieved with the following gradient: A: 1 M (NH4)2SO4, 500 mM NaCl, 100 mM NaH2PO4 pH 7.4 B: 20 mM NaH2PO4, 20% (v/v) Isopropyl alcohol, pH 7.4. 0% B: 0-1 min, 0-95% B: 1-15 min, 95% B: 15-20 min, 95-0% B: 20-23 min, 0% B: 23-25 min, with a flow of 700 uL/min. 15 μg sample where loaded onto the column for each analysis. UV chromatograms were recorded at 220 and 280 nm.

### Charge-Variant Analysis

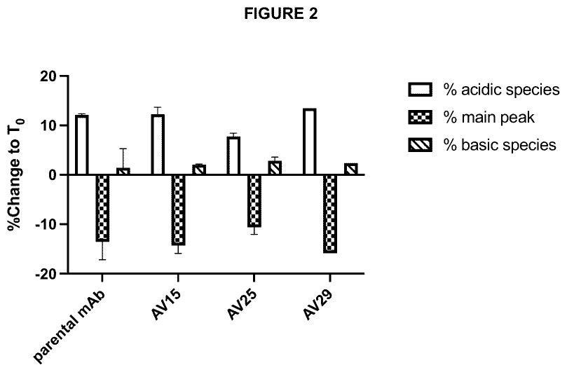

The measurements were conducted on a Vanquish Flex UHPLC System (2.9) with a ProPac Elite WCX 5 μm 4×150 mm column (Thermo Fisher Scientific). Separation of antibody charge variants was performed with the following gradient: A: 1× CX-1 buffer pH 5.6 B: 1× CX-1 buffer pH 10.2 (Thermo Fisher Scientific). 0-100% B: 0-60 min, with a flow of 1 ml/min. 24 μg sample were loaded onto the column for each analysis. UV chromatograms were recorded at 280 nm. Charge variant analysis has been performed as described above. Indicated are changes after incubation for 7 days at 40° C. with respect to day 0 (decrease of main peak in % and increase of acidic and basic species in %) (FIG. 2).

### SDS-PAGE

Samples were prepared for SDS-PAGE by incubation in SDS sample buffer (BioRad) supplemented with 25 mM DTT (95° C., 5 minutes) and separated on a 4-20% Polyacrylamide gel (BioRad 4-20% Mini Protean TGX), followed by Coomassie staining (Thermo Fisher Scientific Imperial Protein Stain).

### ADC Synthesis: General Method for the Conjugation of P5-Based Linker-Payload Constructs to Antibodies to Achieve DARE

50 μl of the respective anti-NaPi2b antibody (parental, AV15, AV18, AV21, AV25, AV29) in a solution of 10.0 mg/ml in P5-conjugation buffer (50 mM Tris, 1 mM EDTA, 100 mM NaCl, pH 8.3 at RT) were mixed with 3.33 μl of a 10 mM TCEP solution in P5-conjugation buffer. Directly afterwards, 1.67 μl of a 40 mM solution of the P5-Exatecan construct dissolved in DMSO were added. The mixture was shaken at 350 rpm and 25° C. for 16 hours. The reaction mixtures were purified by preparative size-exclusion chromatography with a 25 ml Superdex™ 200 Increase 10/300GL (Cytiva, Sweden) and a flow of 0.8 ml/min eluting with sterile PBS (Merck, Germany). The antibody containing fractions were pooled and concentrated by spin-filtration (Amicon® Ultra-2 mL MWCO: 30 kDa, Merck, Germany).

### Binding to Human and Rat NaPi2B Evaluated by Flow Cytometry

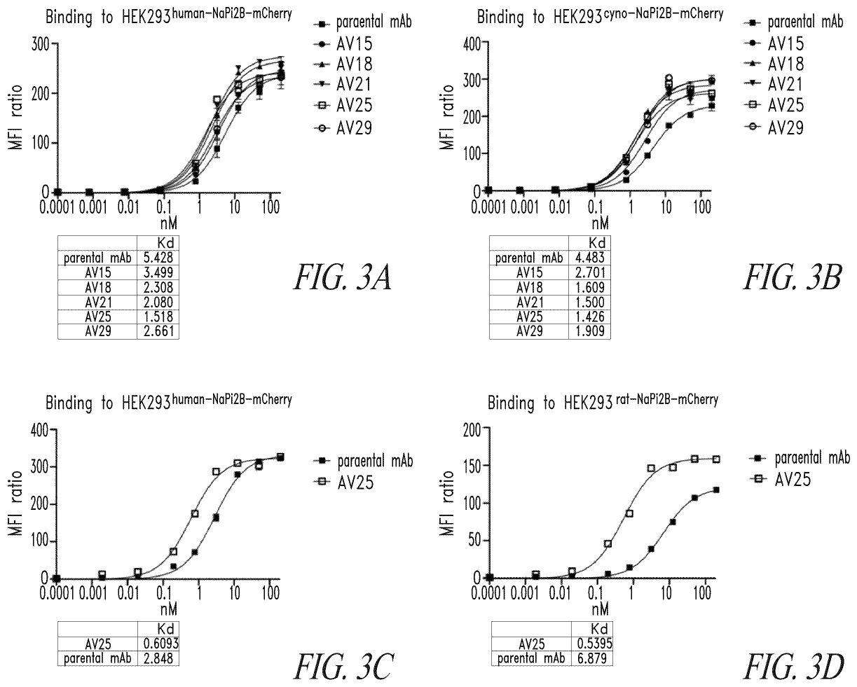

HEK293 cells stably overexpressing human and rat Napi2b were generated by stable integration of a human or rat full-length Napi2b-mCherry-(GGGS)3x-mCherry expression construct under control of a human EF1 promoter and a Puromycin-selection cassette under a CMV promoter into the parental HEK293 cell line. In brief, cells were transfected with the linearized plasmids using Lipfectamine 2000 (Thermo Fisher) and selected with Puromycin antibiotic. Cells were then single cell cloned in 96-well plates via serial dilution under continuous antibiotic selection. Clones were screened for target expression via flow cytometry and mCherry fluorescence via flow cytometry and fluorescence microscopy. Target- and mCherry-positive clones were expanded and used for further experiments. To determine equilibrium binding constants (K_{D}), HEK293 cells stably expressing human (A and C), cynomolgus (B) or rat (D) full-length NaPi2b-mCherry were incubated with antibodies in concentrations ranging from 0.002 to 200 nM and stained with an Alexa-dye-labeled anti-human IgG H+L secondary antibody (Thermo Fisher Scientific) and analyzed by flow cytometry. Binding of antibodies was investigated on mCherry-positive cells. Mean fluorescence intensity (MFI) ratios were normalized to the secondary antibody control. The assay was performed in duplicates and data points were analyzed by a non-linear regression using a one-site specific binding model to derive K_{D} values using Prism 9 software. Graph shows means of n=2±SEM (FIGS. 3A to 3D).

### Binding to Immobilized Human NaPi2B Evaluated by ELISA

Binding of increasing concentrations of parental mAb and AV mAb clones to a purified recombinant immobilized human Napi2b antigen was tested in an ELISA setting.

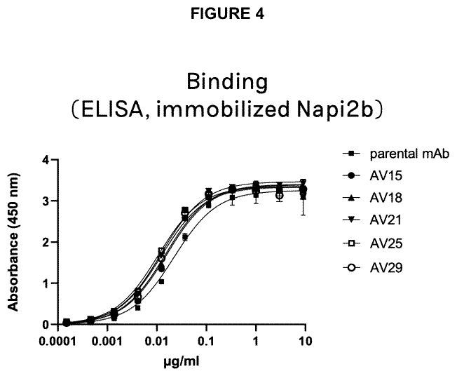

To determine binding of parental and AV mAb clones to Napi2b antigen, flat-bottom surface-treated 96-well-plates (Nunclon, Thermo Fisher Scientific) were coated with 1 μg/mL purified recombinant human Napi2b antigen consisting of Napi2b-extracellular-loop2 expressed as Fc-fusion-6-His-tagged protein in Expi-HEK293 cells. After blocking with 2% bovine serum albumin (Carl Roth) in 1× PBS-Tween 20 (0.05%), increasing antibody concentrations (0.00015, 0.00046, 0.00137, 0.00412, 0.01235, 0.03704, 0.11111, 0.33333, 1.00000, 3.00000, 9.00000 μg/ml) were allowed to bind for 2 hr at room temperature. Bound antibodies were detected by incubation with HRP-conjugated goat anti-human kappa light chain secondary antibody (dilution 1:10000 in blocking solution) for 1 hr at RT. Ultra-TMB (Thermo Fisher, 34028) substrate was added and incubated at room temperature for 15 to 30 min, after which 100 μl/well of 1 M Sulfuric Acid was added. Absorbance at 450 nm was measured within 10 min of addition of acid using a microplate reader Infinite M1000 Pro (Tecan). Apparent dissociation constants (K_{D}) were derived by non-linear regression using a one-site specific binding model using Prism 9 software. K_{D} in μg/ml, 0.02209 parental mAb, 0.01581 AV15, 0.01494 AV18, 0,01248 AV21, 0,01060 AV25, 0.01238 AV29. Graph shows means of n=2±SEM (FIG. 4).

### Internalization Evaluated Via Flow Cytometry

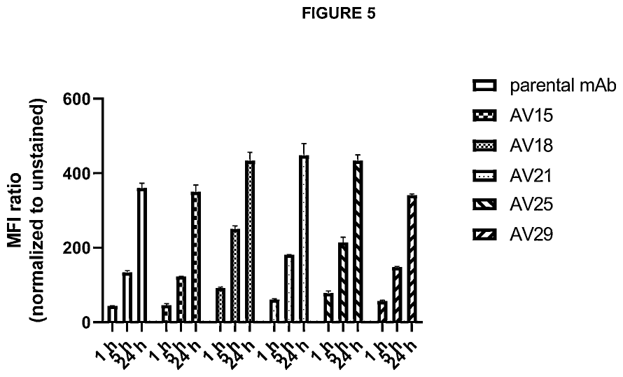

For pHrodo-based investigation of internalization, a goat anti-human IgG FC7 fragment-specific secondary antibody (Jackson ImmunoResearch) was labeled with pHrodo™ Deep Red Antibody Labeling Kit (Thermo Fisher Scientific) according to manufacturer's instructions. Napi2b-positive OVCAR-3 cells were incubated with 5 μg/ml parental mAb and AV mAb clones in presence of equimolar amounts of pHrodo Deep Red-labeled secondary antibody for 1 h, 5 h and 24 h at 37° C. An increase in MFI indicates the presence of AV antibodies in late endosomal and lysosomal compartments. The MFI ratio was determined by deviding the MFI of pHrodo-incubated cells by the MFI of unstained cells (FIG. 5).

### Melting Curves Evaluated by NanoDSF

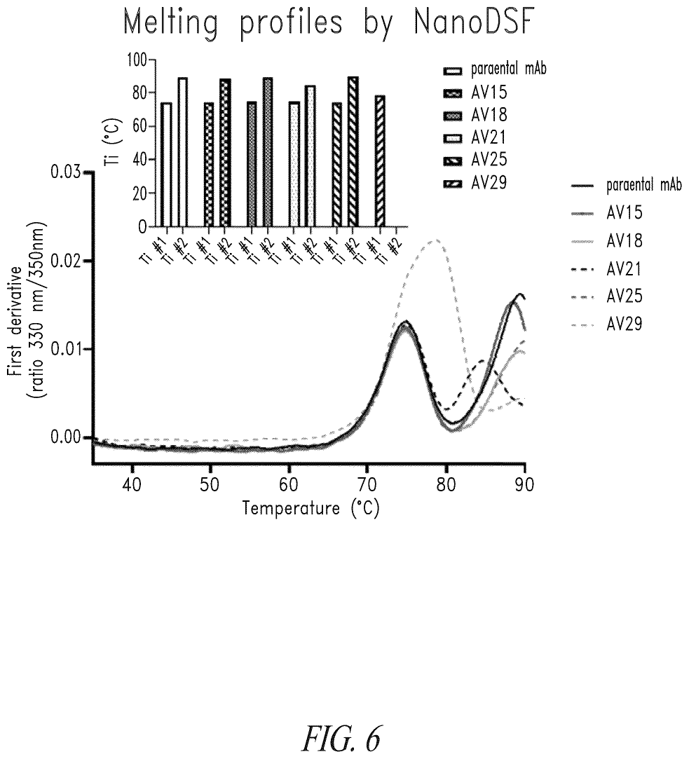

The thermal stability of proteins was determined using nano differential scanning fluorimetry (nanoDSF) that measures temperature-dependent changes in the intrinsic fluorescence of tryptophane and tyrosine residues (Tycho NT.6, NanoTemper Technologies). For this, 1 μM of protein in PBS was absorbed by a capillary that was subsequently placed into the reader. Afterwards, the intrinsic protein fluorescence was measured at 330 nM and 350 nM while incubating at increasing temperatures. Changes in fluorescence signal indicated transitions in the folding state of the proteins and the temperatures at which a transition occurred are named as inflection temperatures (Tᵢ) or also melting temperatures (Tₘ) (Haffke, M. et al., Label-free Thermal Unfolding Assay of G Protein-Coupled Receptors for Compound Screening and Buffer Composition Optimization. 2016) (FIG. 6).

### In Vitro Cytotoxicity Evaluated Via Resazurin Assay

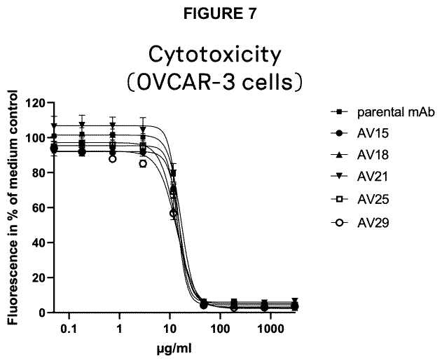

To investigate direct cytotoxicity of ADCs, respective cells were seeded in a 96-well plate (flat bottom, 5000 cells/well, suspended in 100 μl medium) and incubated for 7 days with increasing concentrations of the ADCs in medium (0-3 μg/ml) to generate a dose-response curve. Before viability analysis, the supernatant over the adherent cells was removed and replaced by fresh medium. Killing was analyzed afterwards, using resazurin (Sigma-Aldrich) as the cell viability dye at a final concentration of 55 μM. Fluorescence emission at 590 nM was measured on a Microplate reader Infinite M1000 Pro (Tecan). Cell viability was measured by dividing the fluorescence of ADC-treated cells by the fluorescence from control cells, treated in the same way with medium only. Graph shows means of n=2±SEM (FIG. 7).

### Bystander Killing

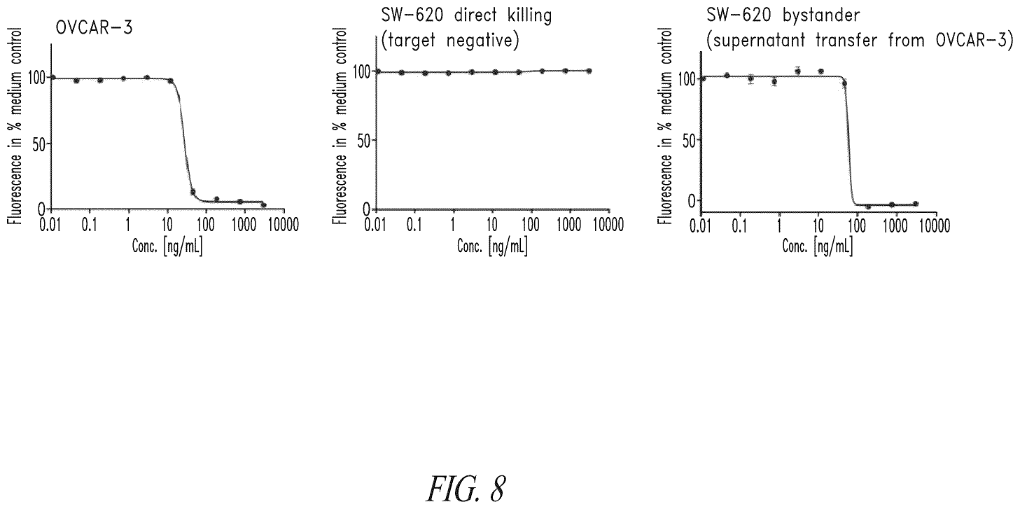

To analyze bystander activity of ADCs on target-negative cells, 20.000 NaPi2B-positive cells (OVCAR-3) were incubated with increasing concentrations of ADCs (0-3 μg/ml). After 5 days, half of the cell culture supernatant volumes was transferred to 5.000 NaPi2B-negative cells (SW-620) and incubated for another 5 days. Killing was analyzed by a resazurin-based viability measurement as described above (FIG. 8).

### In Vitro Inhibition of Topoisomerase-I, by the Delivery of Exatecan Via the ADC

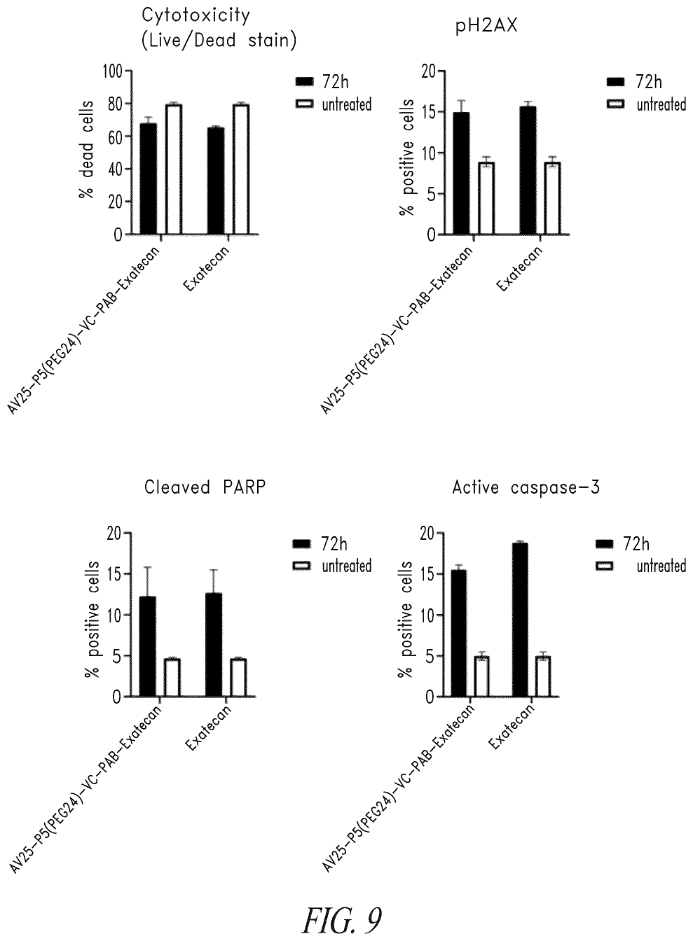

TOPOisomerase-I inhibition by delivery of Exatecan via the TUB-040 ADC induces DNA-damage markers. OVCAR-3 cells were treated with 5 μg/mL TUB-040 or 5 nM free Exatecan for 72 h. Cells were stained with Live/Dead stain and for the DNA-damage markers active caspase-3, cleaved PARP and phosphorylated H2A.X (Ser-139) and analyzed by flow cytometry. Graphs show means of n=2±SEM (FIG. 9).

### In Vivo PK-Evaluation of one ADC

In vivo PK-experiments have been performed with AV25 and AV25-P5(PEG24)-VC-PAB-Exatecan. Female Sprague Dawley rats have been treated with 10 mg/kg of the unconjugated AV25 antibody or the ADC. Blood sampling has been performed after different time points and the ADC amount was quantified in a total antibody and an intact ADC ELISA-assay.

To evaluate the PK of the ADCs in vivo, the total antibody concentration was measured at different time points in serum of ADC-treated SD rats. Total humanized anti-NaPi2B antibody was analyzed in rat serum over the range 2000-15,6 ng/ml. Nunc 96-well plate with (100 μl/well) were coated with NaPi2B diluted in PBS (required concentration: 0,25 μg/ml) and sealed with PCR Foil. Plates were incubated in a fridge to maintain a temperature between 2-8° C. overnight. The coated plates were washed 3× with 300 μl PBST. 200 μl/well of blocking solution (2% Albumin in PBST) was added, the plate was sealed and an incubated at room temperature for 1 hour. The coated plates were washed 3× with 300 μl PBST. 100 μl/well of prepared standards (2000-15,6 ng/ml of the respective ADCs, QCs and test samples were added, the plates were sealed and incubated at room temperature for 1 hour. The plates were washed 3× with 300 μl PBST. 100 μl/well Anti-Human IgG (γ-chain specific)-Peroxidase antibody (dilution 1:60000 in PBS) was added and incubated for 1 h at rt. The plates were washed 3× with 300 μl PBST. 50 μl/well TMB was added, the plates were sealed and incubated at room temperature for 15 min. 50 μl/well of 1 M Sulfuric Acid was added. Using a Tecan Plate Reader, the absorbance at a wavelength of 450 nm was measured.

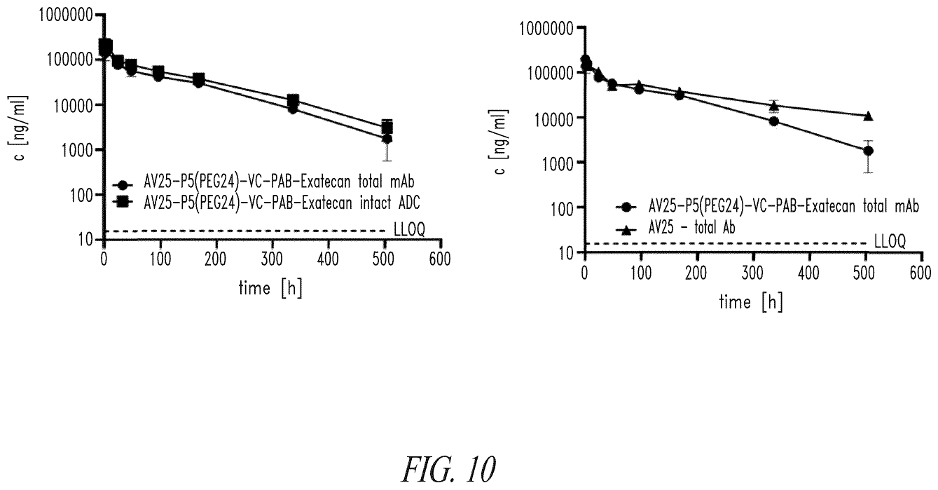

To evaluate the stability of the ADCs in vivo, the intact ADC concentration was measured at different time points in serum of ADC-treated SD rats. Intact ADC was analyzed in rat serum over the range 2000-15,6 ng/ml. Nunc 96-well plate with (100 μl/well) were coated with rabbit anti-Exatecan mAb diluted in PBS (required concentration: 1 μg/ml) and sealed with PCR Foil. Plates were incubated in a fridge to maintain a temperature between 2-8° C. overnight. The coated plates were washed 3× with 300 μl PBST. 200 μl/well of blocking solution (2% Albumin in PBST) was added, the plate was sealed and an incubated at room temperature for 1 hour. The coated plates were washed 3× with 300 μl PBST. 100 μl/well of prepared standards (2000-15,6 ng/ml of the respective ADCs, QCs and test samples were added, the plates were sealed and incubated at room temperature for 1 hour. The plates were washed 3× with 300 μl PBST. 100 μl/well Goat Anti-Human IgG (H+L) Preabsorbed (dilution 1:25000 in PBS was added and incubated for 1h at rt. The plates were washed 3× with 300 μl PBST. 100 μl/well TMB was added, the plates were sealed and incubated at room temperature for 10 min. 100 μl/well of 1 M Sulfuric Acid was added. Using a Tecan Plate Reader, the absorbance at a wavelength of 450 nm was measured (FIG. 10).

### Linker-Payload Synthesis

### General Method 2 for the Synthesis of PEGylated P5 Building Blocks Via the Staudinger Phosphonite Reaction

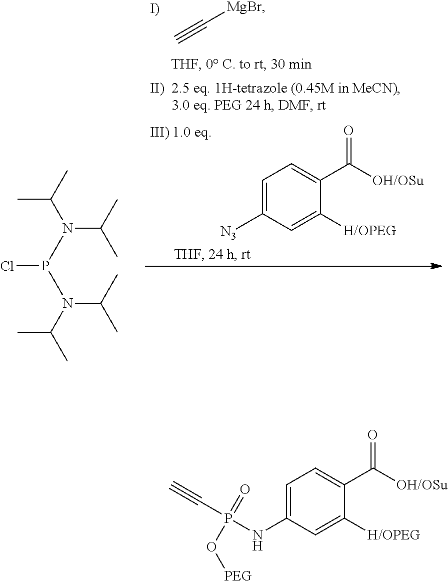

A 25-m1 Schlenk flask was charged with 267 mg bis(diisopropylamino)chlorophosphine (1.00 mmol, 1.00 eq.) under an argon atmosphere, cooled to 0° C. and 2.20 mL ethynylmagnesium bromide solution (0.5 M in THF, 1.10 mmol, 1.10 eq.) was added drop wise. The yellowish solution was allowed to warm to room temperature and stirred for further 30 minutes. 3.00 mmol (3.0 eq.) of the desired PEG-alcohol, dissolved in 5.56 mL 1H tetrazole solution (0.45 M in MeCN, 2.50 mmol, 2.50 eq.) were added and the white suspension was stirred overnight at room temperature. The formation of the desired phosphonite was monitored by ³¹P-NMR. 1.0 mmol (1.0 eq.) of the desired azide dissolved in 2 mL of DMF, THF or MeCN was added and the suspension further stirred for 24h at room temperature. The crude reaction mixture was purified using preparative HPLC.

### P5(PEG12)-OSu

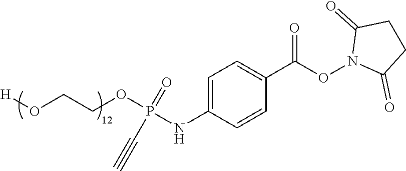

The title compound was synthesized in accordance to general Method 2 from 19.5 mg bis(diisopropylamino)chlorophosphine (73 μmol, 1.00 eq.), 146 μL ethynylmagnesium bromide solution (0.5 M in THF, 73 μmol, 1.00 eq.), 100 mg of dodecaethylene glycol (183 μmol, 2.50 eq), 400 μL 1H-tetrazole solution (0.45 M in MeCN, 183 μmol) and 19 mg 4-azidobenzoic-acid-N-hydroxysuccinimide ester (73 μmol, 1.00 eq.). The product was obtained as colourless oil after preparative HPLC (Method D) and lyophilization. (42.5 mg, 50 μmol, 68%). ¹H NMR (300 MHz, Acetonitrile-d3) δ8.06 (d, J =8.7 Hz, 2H), 7.32 (d, J =8.8 Hz, 2H), 4.40-4.14 (m, 2H), 3.79-3.69 (m, 2H), 3.66-3.47 (m, 40H), 3.21 (d, J =13.1 Hz, 1H), 2.86 (s, 4H), 1.30 (m, 2H), 1.13-0.79 (m, 2H). 13 0 NMR (151 MHz, CDCl₃) 6 169.77, 169.46, 161.66, 161.47, 152.75, 146.09, 132.90, 132.24, 117.82, 113.97, 113.29, 89.25, 88.92, 77.27, 77.06, 76.85, 74.69, 72.57, 71.19, 70.62, 70.54, 70.51, 70.47, 70.44, 70.36, 70.27, 70.20, 69.74, 69.70, 68.14, 65.77, 65.73, 61.63, 61.60, 40.72, 30.34, 25.68. ³¹P NMR (122 MHz, Acetonitrile-d₃) δ-10.87. HRMS C₃₇H₆₀N₂O₁₉P⁺ calc.: 851.3573 [M+H]⁺, 851.3571.

### P5(PEG12)-COON

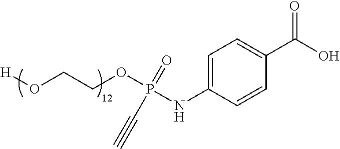

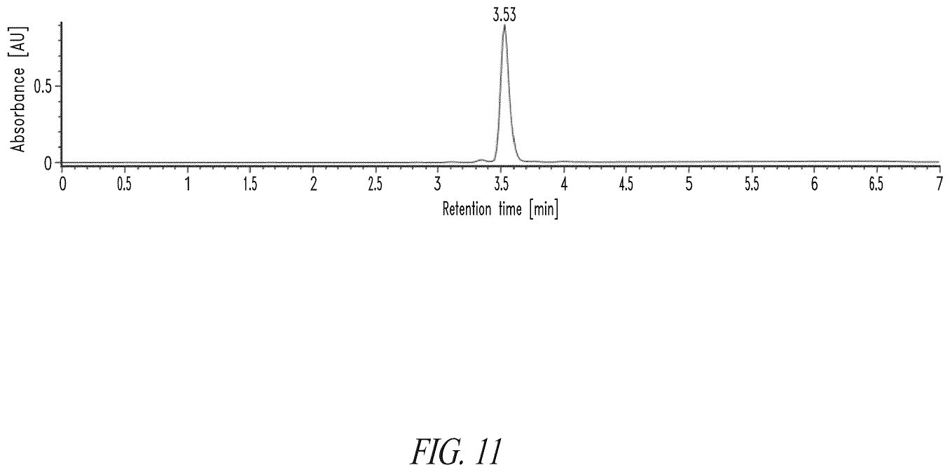

The title compound was synthesized in accordance to general Method 2 from 40 mg bis(diisopropylamino)chlorophosphine (150 μmol, 1.00 eq.), 360 μL ethynylmagnesium bromide solution (0.5 M in THF, 180 μmol, 1.2 eq.), 245 mg of PEG12 (450 μmol, 3.0 eq), 0.83 mL 1H-tetrazole solution (0.45 M in MeCN, 450 μmol, 2.5 eq.) and 39 mg 4-azidobenzoic-acid (150 μmol, 1.00 eq.). The product was obtained as colourless oil after preparative HPLC (Method D) and lyophilization. (25 mg, 34 μmol, 23%). HR-MS for C₃₃H₅₇N₁₆P⁺[M+H]⁺ calcd.: 754.3410, found 754.3398 (UV-trace in FIG. 11).

### P5(PEG24)-OSu

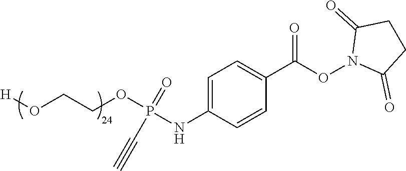

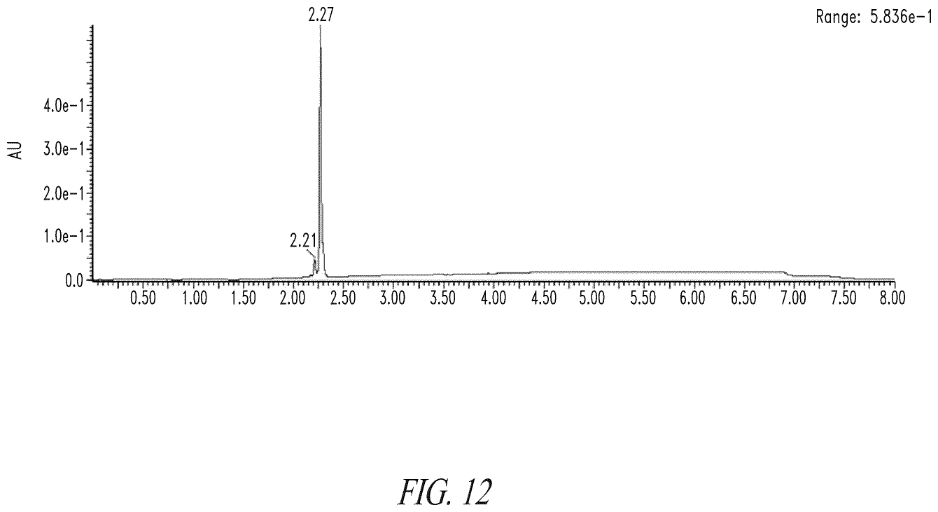

The title compound was synthesized in accordance to general Method 2 from 41 mg bis(diisopropylamino)chlorophosphine (159 μmol, 1.00 eq.), 370 μL ethynylmagnesium bromide solution (0.5 M in THF, 185 μmol, 1.2 eq.), 450 mg of PEG24 (388 μmol, 2.50 eq), 1.02 mL 1H-tetrazole solution (0.45 M in MeCN, 466 μmol, 3.0 eq.) and 40 mg 4-azidobenzoic-acid-N-hydroxysuccinimide ester (155 μmol, 1.00 eq.). The product was obtained as colourless oil after preparative HPLC (Method D) and lyophilization. (79 mg, 57 μmol, 37%). MS for C₆₁H₁₀₉N₂O₃₀P²⁺ [M+2H]²⁺ calcd.: 690.3396, found 690.81. (UV-trace in FIG. 12).

### NH2-VC-PAB-Exatecan TFA Salt

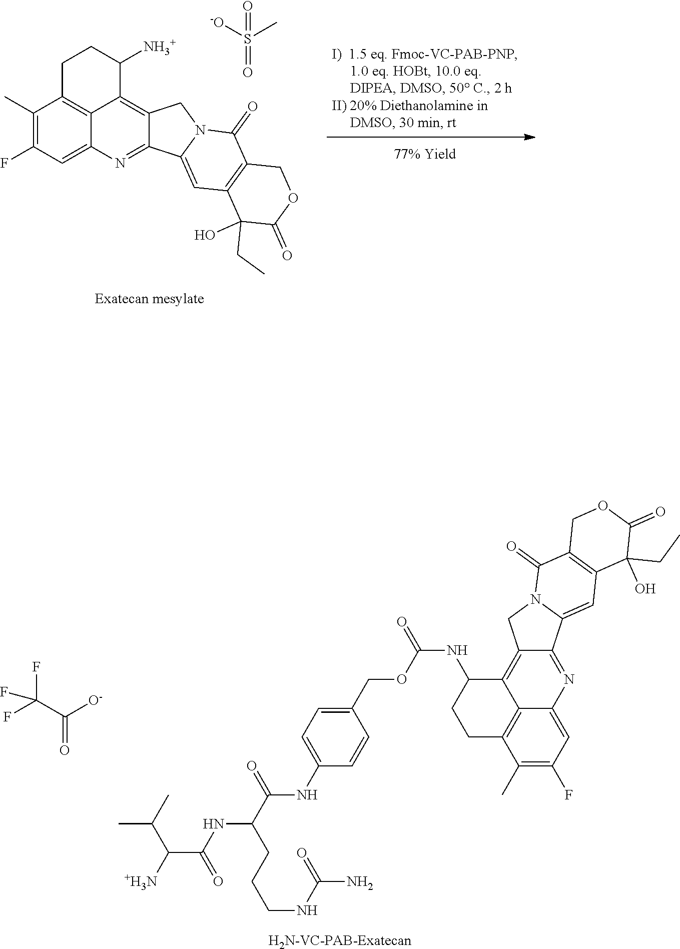

A screw-cap-vial was charged with 34.3 mg of Exatecan Mesylate (0.0645 mmol, 1.0 eq.) and suspended in 645 μL of dry DMSO. 241 μL of a solution of a 0.4 mol/L solution of Fmoc-VC-PAB-PNP in dry DMSO (0.0967 mmol, 1.5 eq.), 64,5 μL of a 1 mol/L solution of HOBt hydrate in dry DMSO (0.0645 mmol, 1.0 eq.) and 113 μL of DIPEA were added (0.645 mmol, 10.0 eq.). The yellow solution was stirred for 2 h at 50° C. Afterwards, 425 μL of a solution of 50% Diethanolamine in dry DMSO (w/w) was added and the reaction mixture was allowed to stir at room temperature for another 30 minutes. 1.5 ml MeCN and 2.5 mL H₂O added and the yellow solution was directly purified by preparative HPLC, using Method D. After lyophilization, 47.3 mg (76.7%, 0.0495 mmol) of a yellowish solid were obtained as TFA-salt.

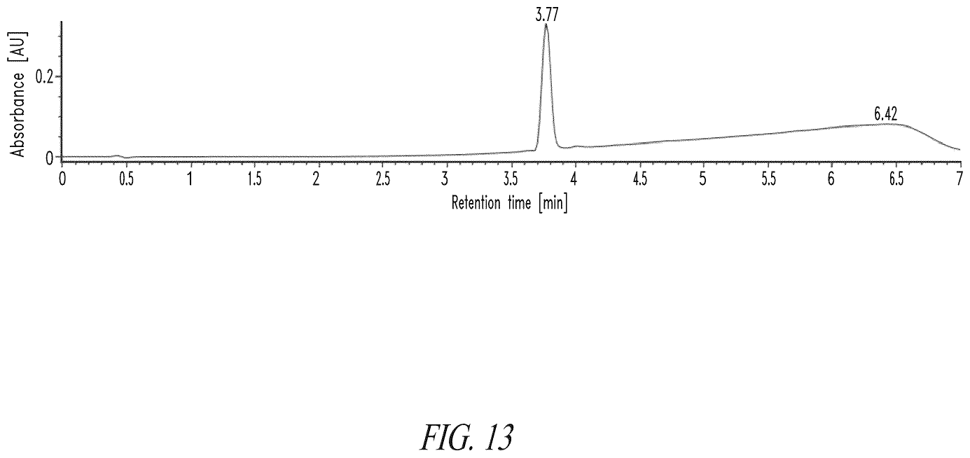

HR-MS for C₄₃H₅₀FN₈O₉⁺ [M+H]⁺ calcd.: 841.3680, found 841.3696 (UV-trace in FIG. 13).

### NH2-VA-PAB-Exatecan TFA Salt

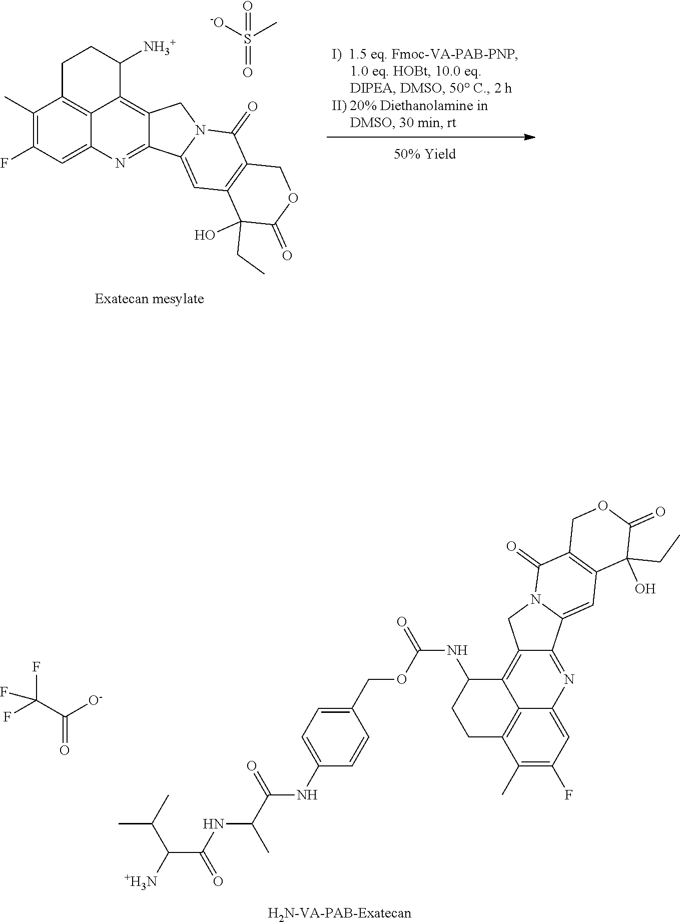

A screw-cap-vial was charged with 1.23 mg of Exatecan Mesylate (0.00232 mmol, 1.0 eq.) and suspended in 23 μL of dry DMSO. 8.7 μL of a solution of a 0.4 mol/L solution of Fmoc-VA-PAB-PNP in dry DMSO (0.00348 mmol, 1.5 eq.), 2.3 μL of a 1 mol/L solution of HOBt hydrate in dry DMSO (0.00232 mmol, 1.0 eq.) and 4 μL of DIPEA were added (0.0232 mmol, 10.0 eq.). The yellow solution was stirred over night at room temperature. Afterwards, 15 μL of a solution of 50% Diethanolamine in dry DMSO (w/w) was added and the reaction mixture was allowed to stir at room temperature for another 30 minutes. 1.5 ml MeCN and 2.5 mL H₂O added and the yellow solution was directly purified by preparative HPLC, using Method C. After lyophilization, 1.01 mg (50.0%, 0.00116 mmol) of a yellowish solid were obtained as TFA-salt.

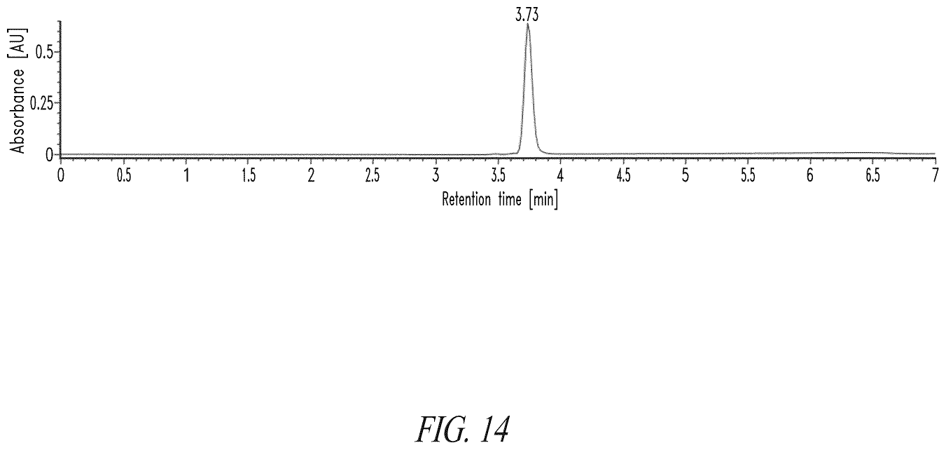

HR-MS for C₄₀H₄₄FN₆O₈⁺ [M+H]⁺ calcd.: 755.3200, found 755.3201. (UV-trace in FIG. 14).

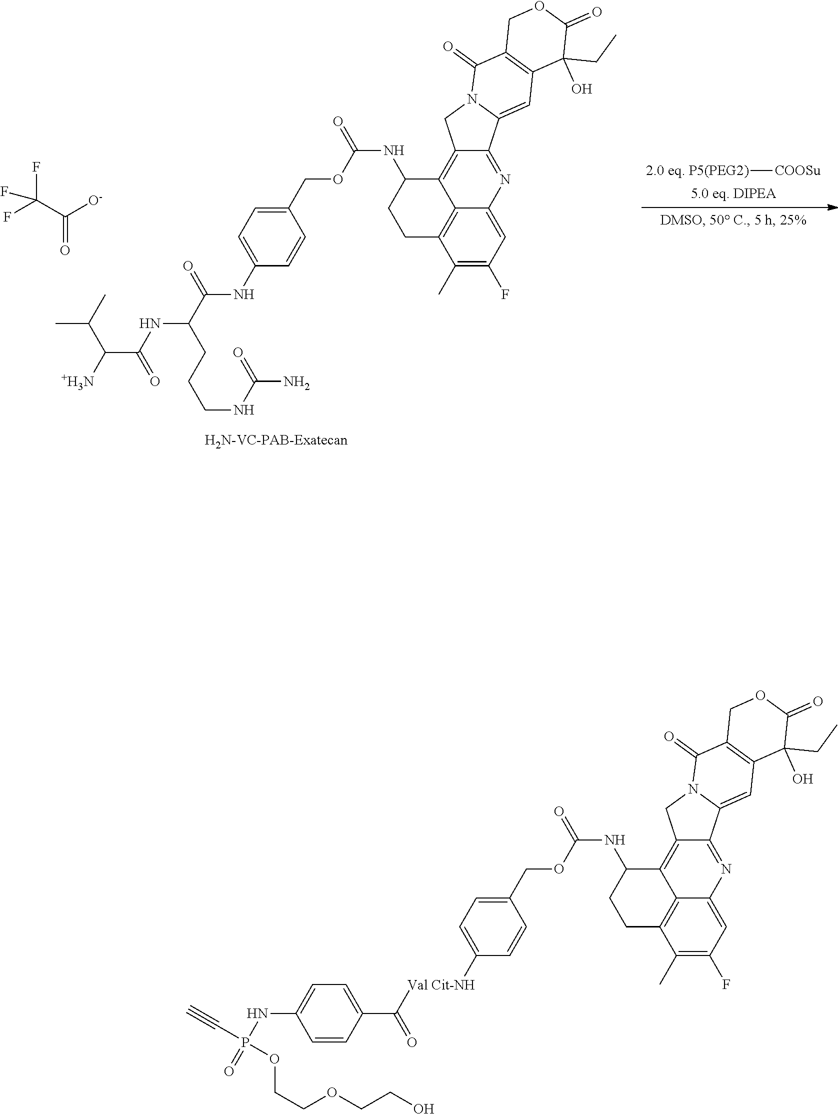

### P5(PEG2)-VC-PAB-Exatecan

A screw-cap-vial was charged with 23,4 μL of a 200 mM solution of NH2-VC-PAB-Exatecan TFA salt in dry DMSO (0.00468 mmol, 1.0 eq.), 46,8 μL of a 200 mM solution of 2-(2-Hydroxyethoxy)ethyl-N-(4-benzoic-acid-N-hydroxysuccinimideester)-P-ethynyl phosphonamidate (P5(PEG2)-COOSu, 0.00936 mmol, 2.0 eq.) and 4.08 μL DIPEA (0.0234 mmol, 5.0 eq.). The solution was shaken for 5 hours at 50° C., cooled to room temperature, 1.5 ml MeCN and 2.5 mL H₂O were added and the solution was directly purified by preparative HPLC, using Method C. After lyophilization, 1.33 mg (25.0%, 0.00117 mmol) of a yellowish solid were obtained.

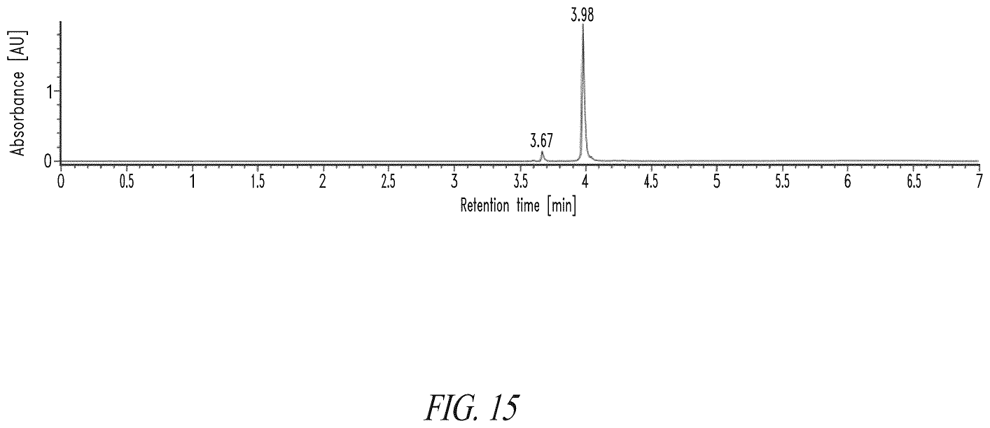

HR-MS for C₅₆H₆₄FN₉O₁₄P⁺ [M+H]⁺ calcd.: 1136.4289, found 1136.4306. (UV-trace in FIG. 15).

### P5(PEG12)-VC-PAB-Exatecan

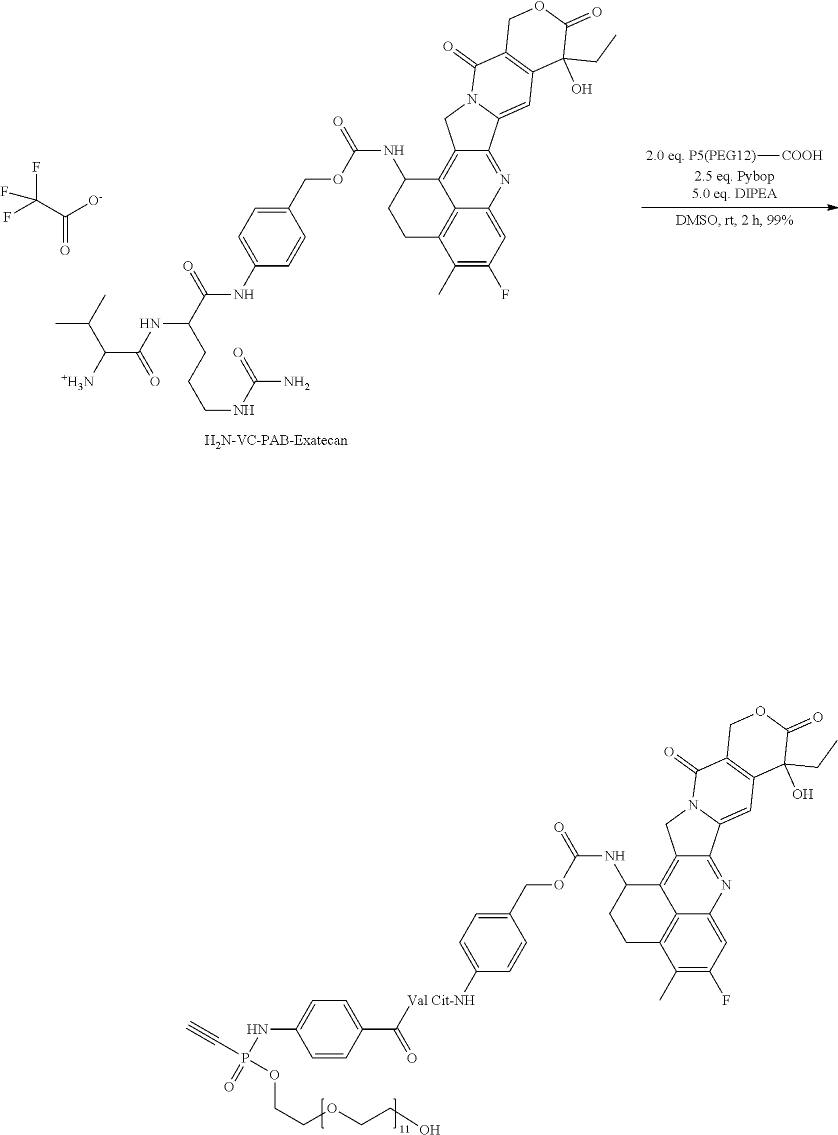

A screw-cap-vial was charged with 51 μL of a 200 mM solution of NH2-VC-PAB-Exatecan TFA salt in dry DMSO (0.0102 mmol, 1.0 eq.), 102 μL of a 200 mM solution of PEG12-N-(4-benzoic-acid)-P-ethynyl phosphonamidate (P5(PEG12)-COOH, 0.0204 mmol, 2.0 eq.) in dry DMSO, 102 μL of a 250 mM solution of Pybop (0.0255 mmol, 2.5 eq.) in dry DMSO and 8.89 μL DIPEA (0.051 mmol, 5.0 eq.). The solution was shaken for 2 hours at room temperature, 1.5 ml MeCN and 2.5 mL H₂O were added and the solution was directly purified by preparative HPLC, using Method D. After lyophilization, 15.91 mg (99.0%, 0.0101 mmol) of a yellowish solid were obtained.

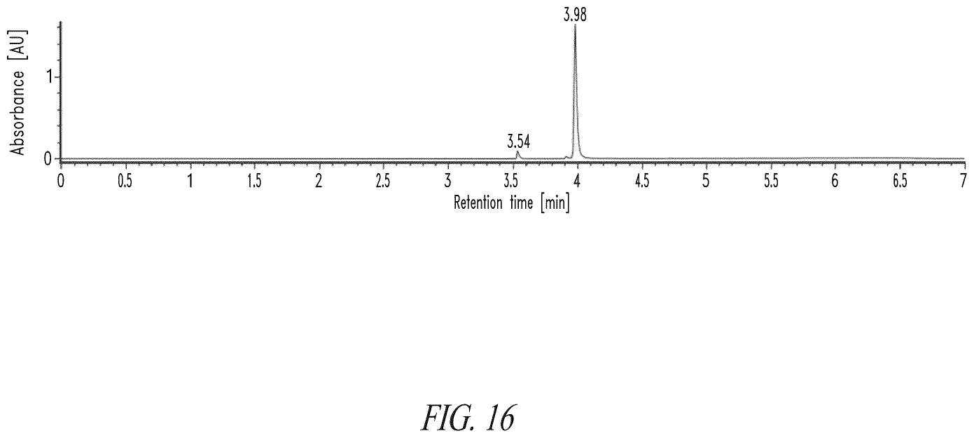

HR-MS for C₇₆H₁₀₅FN₉O₂₄P²⁺ [M+H]²⁺ calcd.: 788.8492, found 788.8485. (UV-trace in FIG. 16).

### P5(PEG24)-VC-PAB-Exatecan

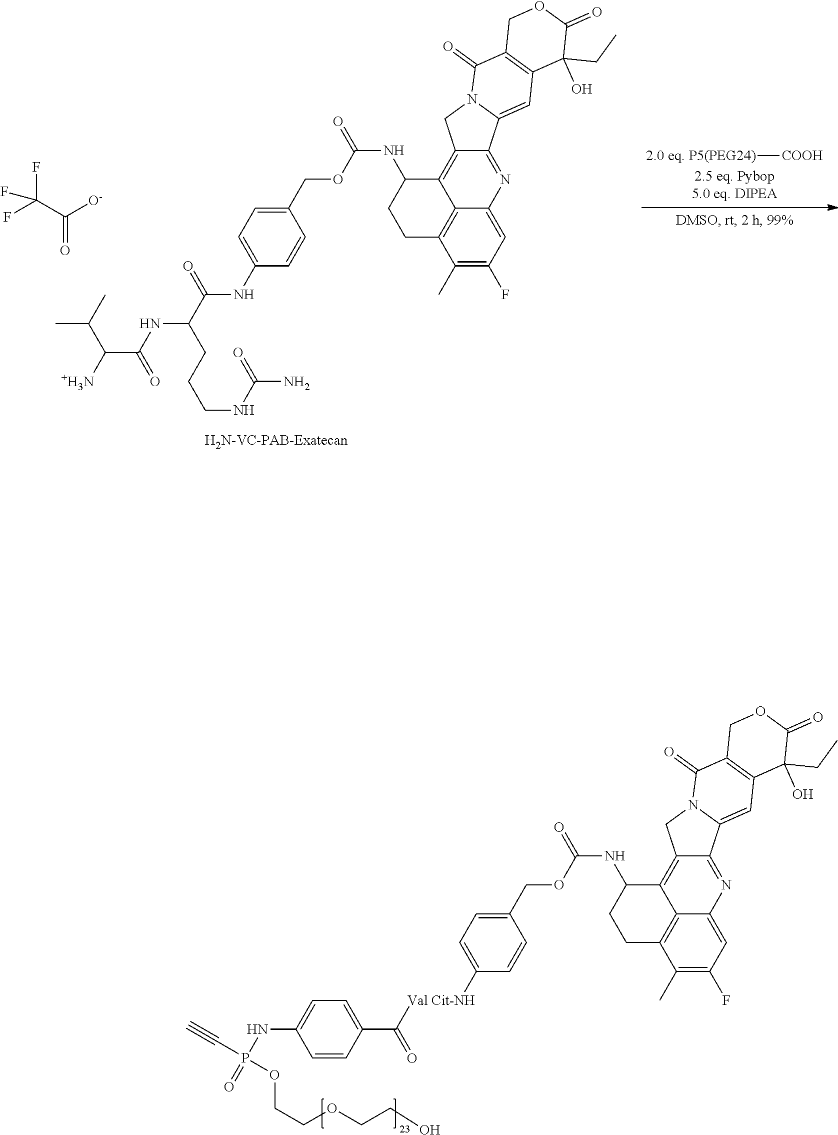

A screw-cap-vial was charged with 102 μL of a 200 mM solution of NH2-VC-PAB-Exatecan TFA salt in dry DMSO (0.0204 mmol, 1.0 eq.), 204 μL of a 200 mM solution of (P5(PEG24)-OSu, 0.0408 mmol, 2.0 eq.) in dry DMSO, and 17.78 μL DIPEA (0.102 mmol, 5.0 eq.). The solution was shaken over night at room temperature, 1.5 ml MeCN and 2.5 mL H₂O were added and the solution was directly purified by preparative HPLC, using Method D. After lyophilization, 25,76 mg (60.0%, 0.01224 mmol) of a yellowish solid were obtained.

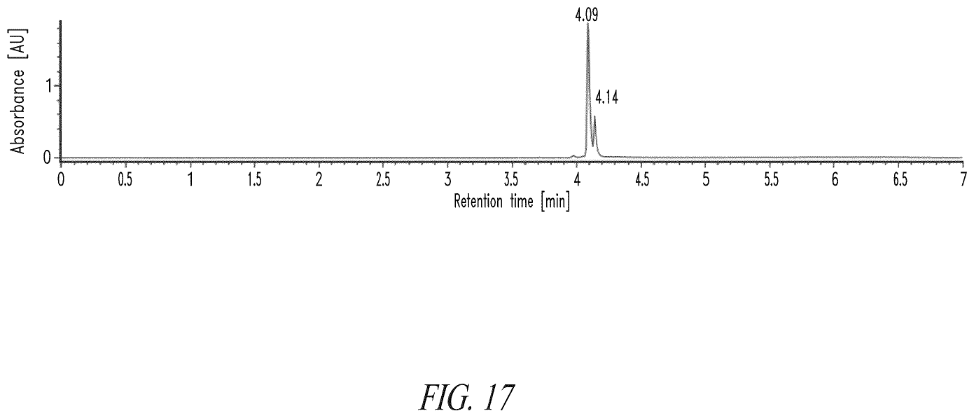

HR-MS for C₁₀₀H₁₅₃FN₉O₃₆P²⁺ [M+H]²⁺ calcd.: 1053.5081, found 1053.50833. (UV-trace in FIG. 17).

### P5(PEG12)-VA-PAB-Exatecan

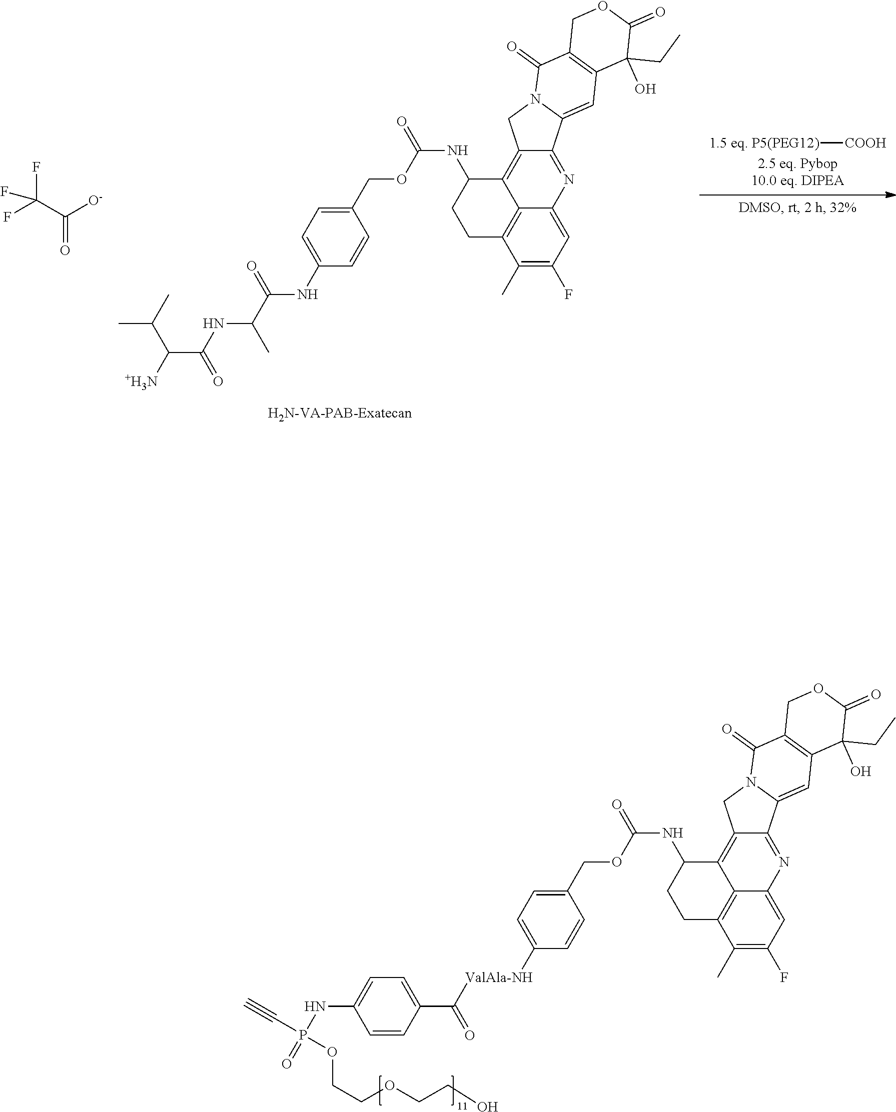

A screw-cap-vial was charged with 11.6 μL of a 100 mM solution of NH2-VA-PAB-Exatecan TFA salt in dry DMSO (0.00116 mmol, 1.0 eq.), 8.7 μL of a 200 mM solution of PEG12-N-(4-benzoic-acid)-P-ethynyl phosphonamidate (P5(PEG12)-COOH, 0.00174 mmol, 1.5 eq.) in dry DMSO, 11.6 μL of a 200 mM solution of Pybop (0.00232 mmol, 2.0 eq.) in dry DMSO and 2.02 μL DIPEA (0.0116 mmol, 10.0 eq.). The solution was shaken for 2 hours at room temperature, 1.5 ml MeCN and 2.5 mL H₂O were added and the solution was directly purified by preparative HPLC, using Method C. After lyophilization, 0.56 mg (32,2%, 0.000375 mmol) of a yellowish solid were obtained.

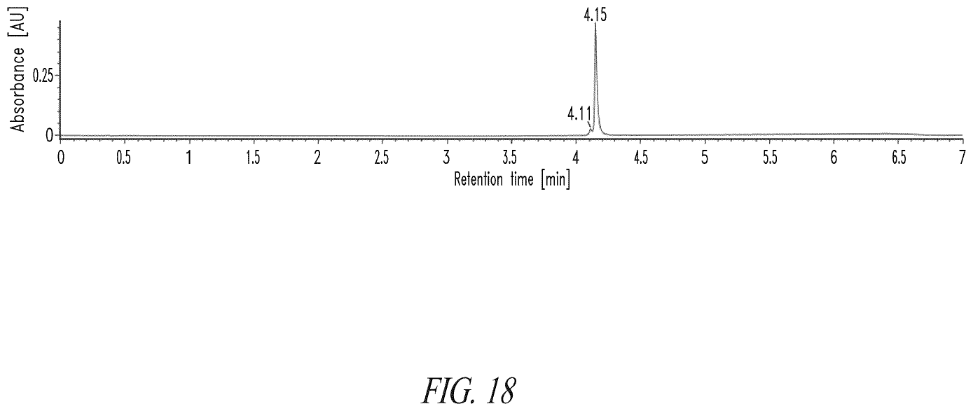

HR-MS for C₇₃H₉₉FN₇O₂₃P²⁺ [M+H]²⁺ calcd.: 745.8252, found 745.8255. (UV-trace in FIG. 18).

### P5(PEG12)-Exatecan

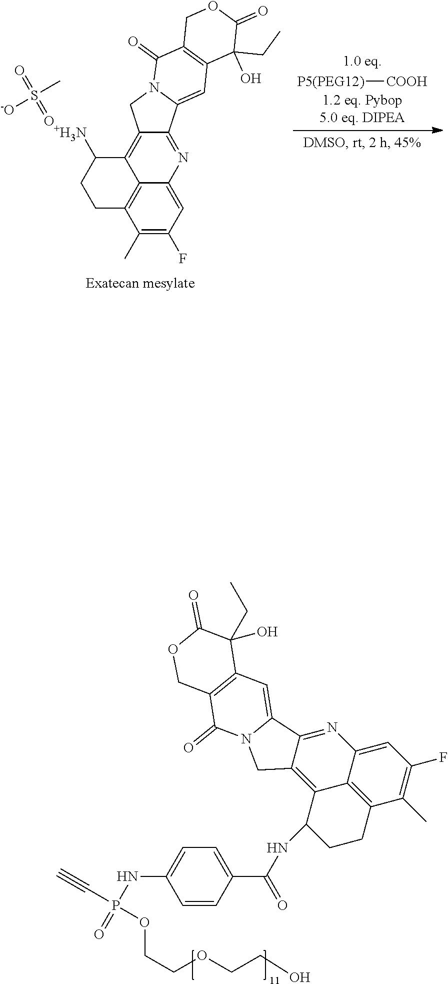

A screw-cap-vial was charged with 50 μL of a 100 mM suspension of Exatecan Mesylate in dry DMSO (0.005 mmol, 1.0 eq.), 20 μL of a 250 mM solution of PEG12-N-(4-benzoic-acid)-P-ethynyl phosphonamidate (P5(PEG12)-COOH, 0.005 mmol, 1.0 eq.) in dry DMSO, 20 μL of a 300mM solution of Pybop (0.006 mmol, 1.2 eq.) in dry DMSO and 4.33 μL DIPEA (0.025 mmol, 5.0 eq.). The solution was shaken for 2 hours at room temperature, 1.5 ml MeCN and 2.5 mL H₂O were added and the solution was directly purified by preparative HPLC, using Method C. After lyophilization, 2.63 mg (45.0%, 0.0023 mmol) of a yellowish solid were obtained.

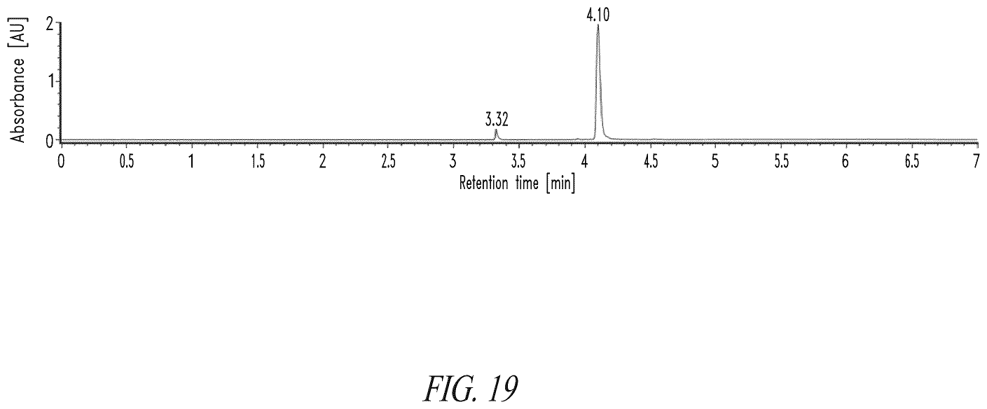

HR-MS for C₅₇H₇₇FN₄O₁₉P⁺ [M+H]⁺ calcd.: 1171.4899, found 1171.4852. (UV-trace in FIG. 19).

### mAb and ADC Characterization

Analytical Overview of the Synthesized mAbs and ADCs from P5(PEG24)-VC-PAB-Exatecan (FIG. 20)

% HMWS have been measured by analytical size-exclusion chromatography, as described above. % indicates the area of peaks with a lower retention time with respect to the monomeric species. Charge variant analysis has been performed as described above. Indicated are changes after incubation for 7 days at 40° C. with respect to day 0. ADC Retention time has been measured by Hydrophobic interaction Chromatography, as described above. The Ti has been obtained from the melting curves, measured by nano DSF, as described above. The binding affinity has been measured by flow cytometry (FACS on OVCAR-3 cells, or via ELISA on the isolated protein as described above. The Cytotoxicity has been assessed via a Resazurin assay, as described above. Internalization is stated as % of MFI ratio for pH rodo after 5-6 hours with respect to timepoint 0, as described above.

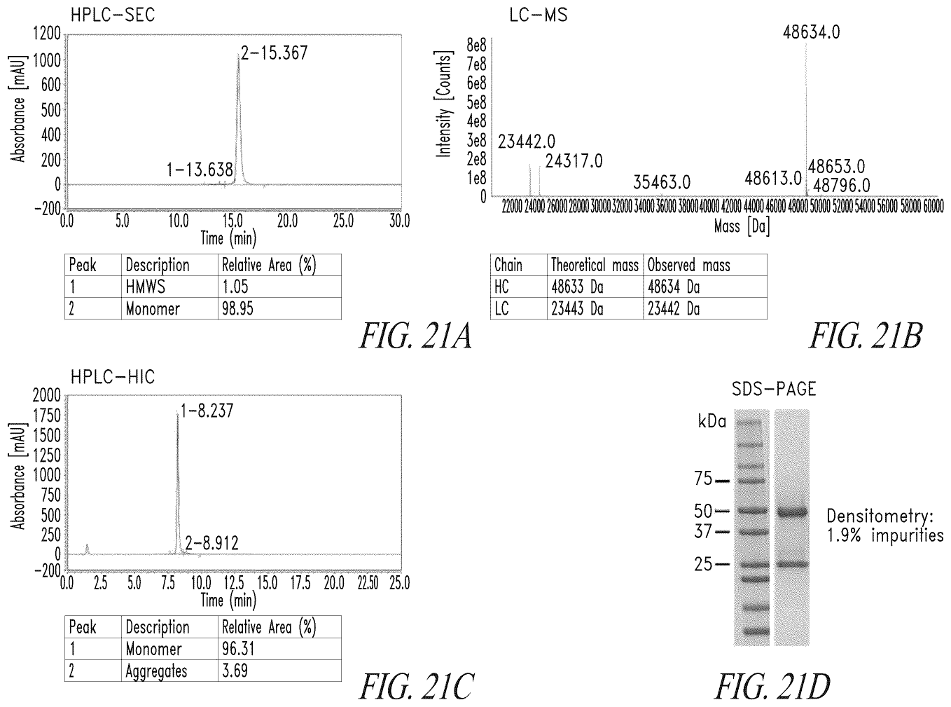

### Analytical Raw Data for an Exemplary Monoclonal Antibody (FIGS. 21A to 21D).

(A-D) Analytical characterization of one of the described mAbs. The antibody was expressed in Expi-CHO cells and purified via Protein A chromatography, as described above. The mAb has been analyzed via HLPC-SEC (A), LC-MS (B), HLPC-HIC (C) and reducing SDS-PAGE (D).

LC/MS Analysis of the Antibody Clones

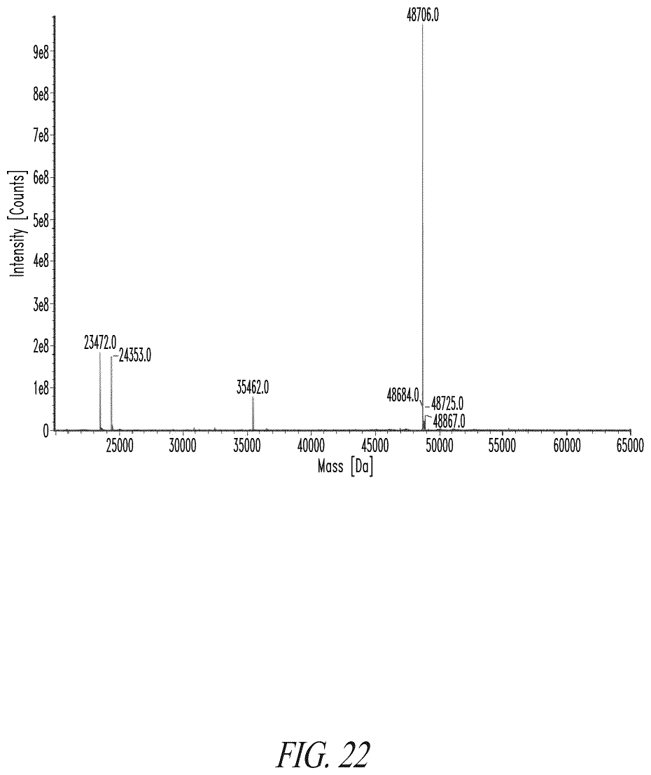

### Parental (FIG. 22)

Calculated LC: 23473, found: 23472 ; Calculated HC: 48705, found: 48706.

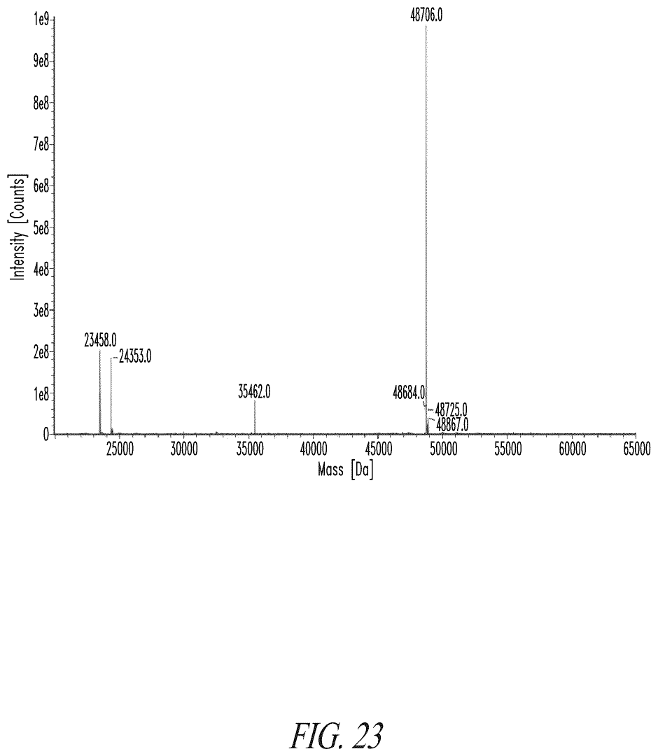

### AV15 (FIG. 23)

Calculated LC: 23459, found: 23458; Calculated HC: 48705, found: 48706.

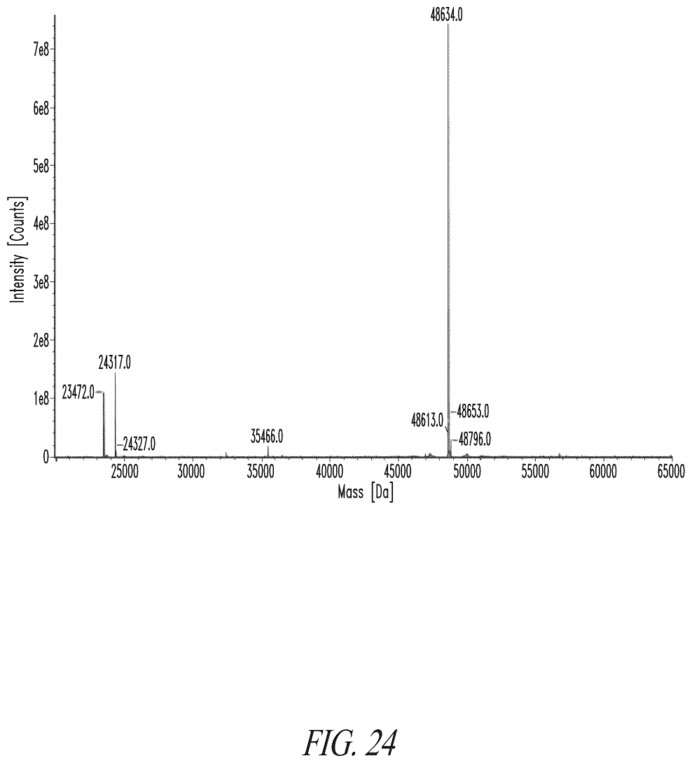

### AV18 (FIG. 24)

Calculated LC: 23473, found: 23472; Calculated HC: 48632, found: 48634.

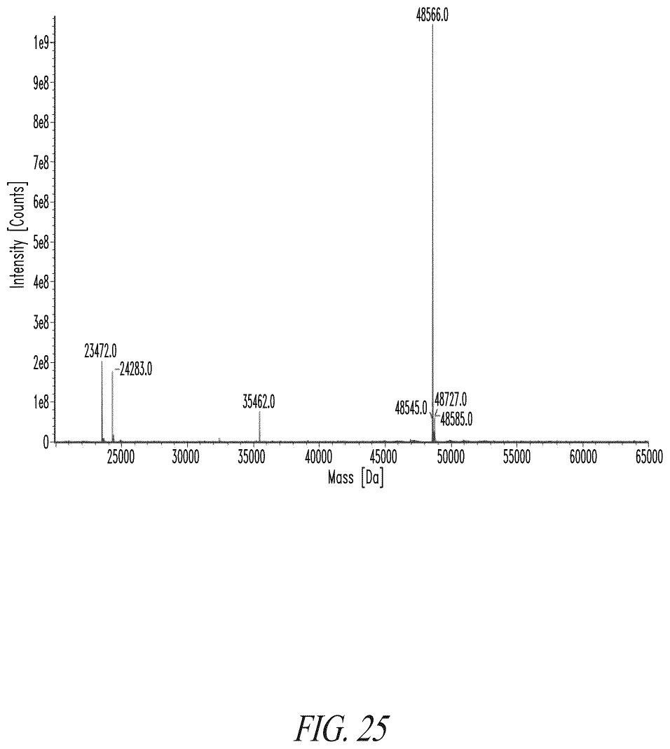

### AV21 (FIG. 25)

Calculated LC: 23473, found: 23472; Calculated HC: 48564, found: 48566.

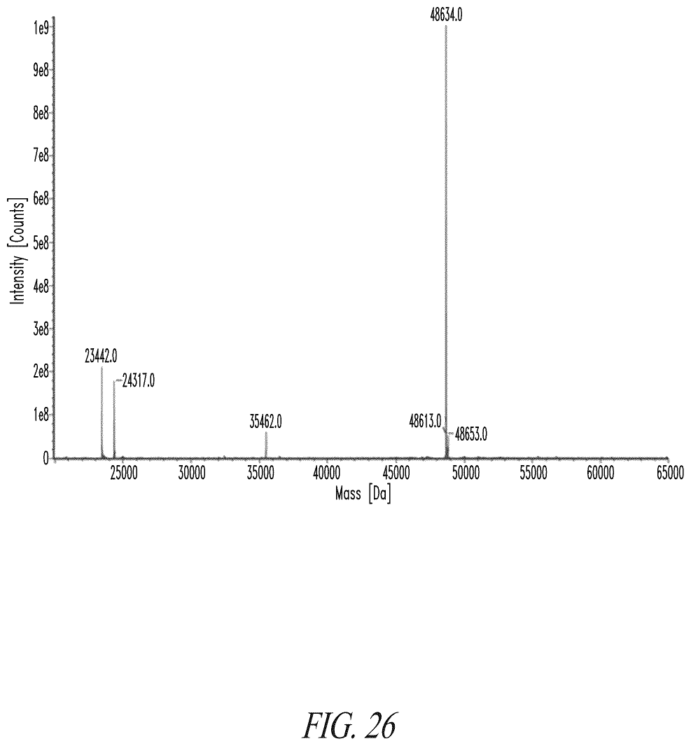

### AV25 (FIG. 26)

Calculated LC: 23443, found: 23442 ; Calculated HC: 48632, found: 48634.

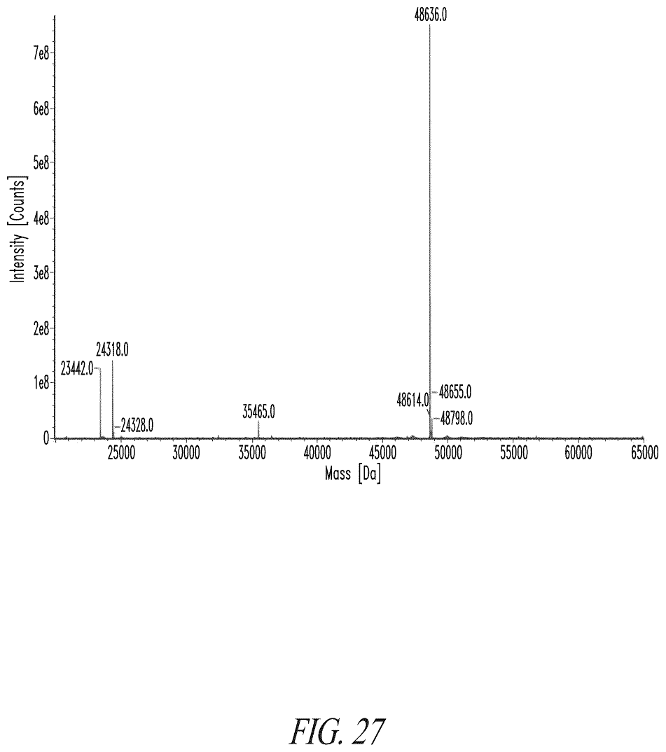

### AV29 (FIG. 27)

Calculated LC: 23443, found: 23442; Calculated HC: 48634, found: 48636.

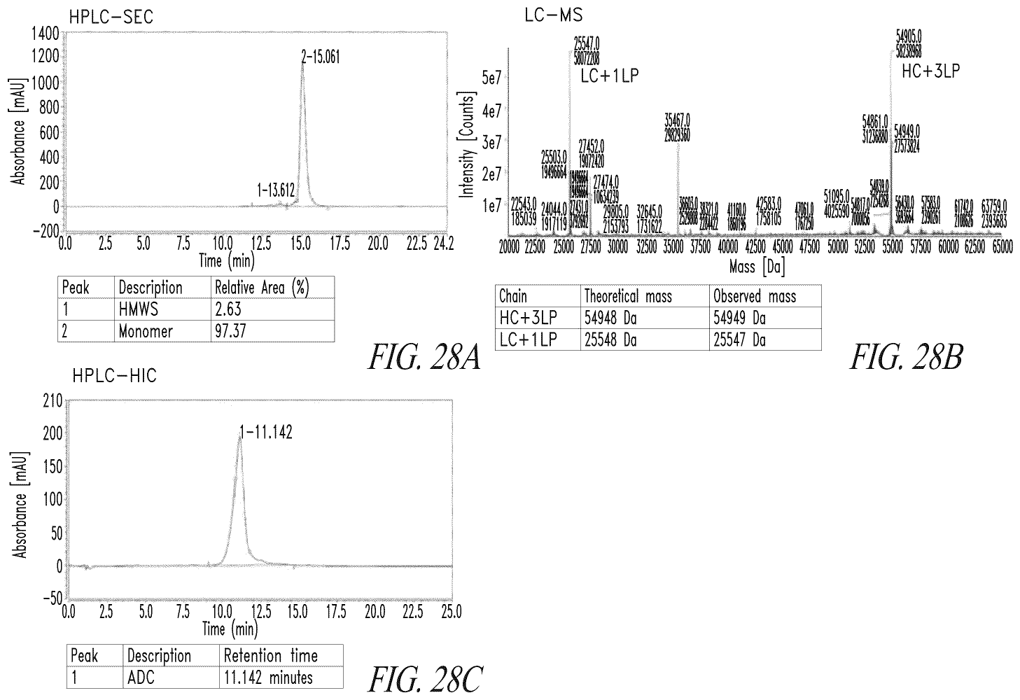

### Analytical Raw Data for an Exemplary ADC (FIGS. 28A to 28C)

FIGS. 28A to 28C. Analytical characterization of AV25-P5(PEG24)-VC-PAB-Exatecan (DAR8), synthesized and purified as described above. A) Analytical size-exclusion chromatography (FIG. 28A), B) LC-MS of the ADC product (FIG. 28B), C) analytical hydrophobic interaction chromatography after conjugation (FIG. 28C). The data indicates that the ADC is fully conjugated to DAR8 with only low amounts of aggregates being present after purification.

### LC/MS Analysis of the ADCs

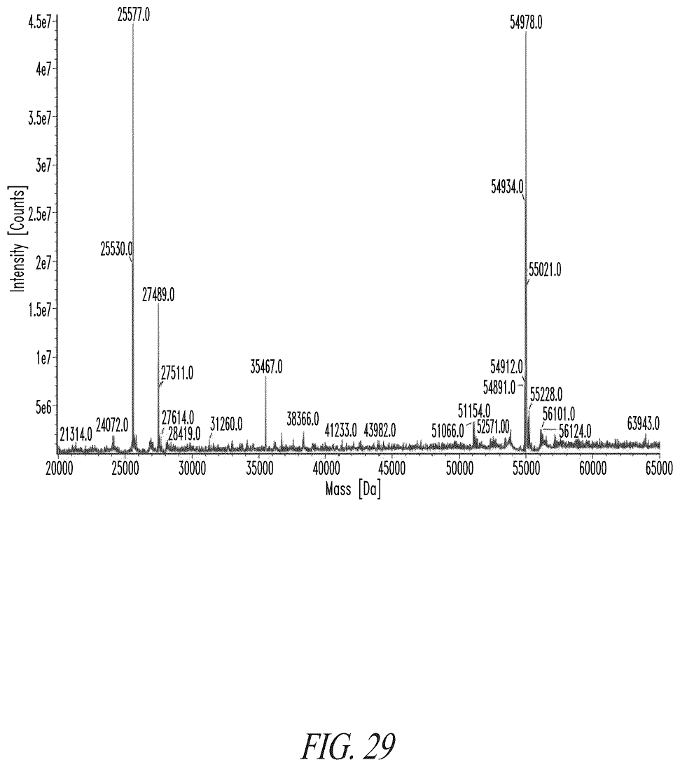

### Parental-P5(PEG24)-VC-PAB-Exatecan DAR8 (FIG. 29)

Calculated LC: 25579, found: 25577 ; Calculated HC: 55023, found: 55021.

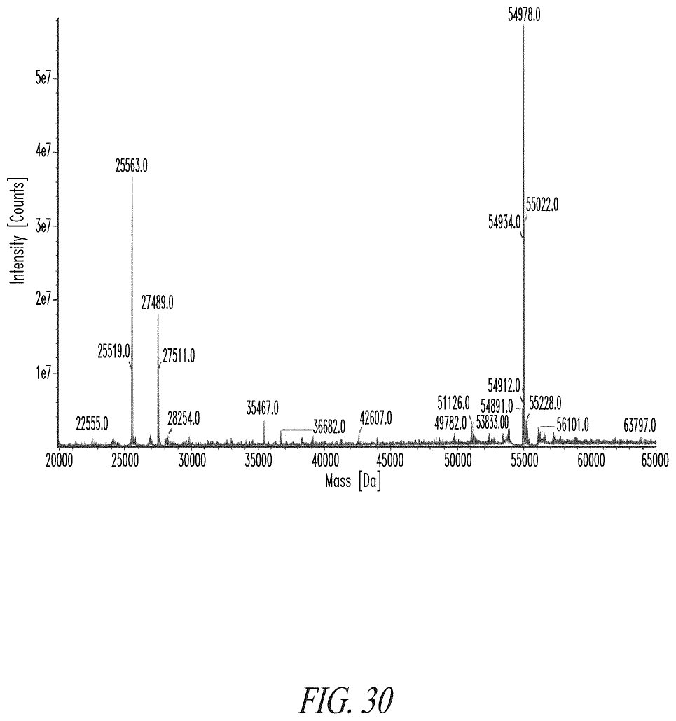

### AV15-P5(PEG24)-VC-PAB-Exatecan DAR8 (FIG. 30)

Calculated LC: 25565, found: 25563 ; Calculated HC: 55023, found: 55022.

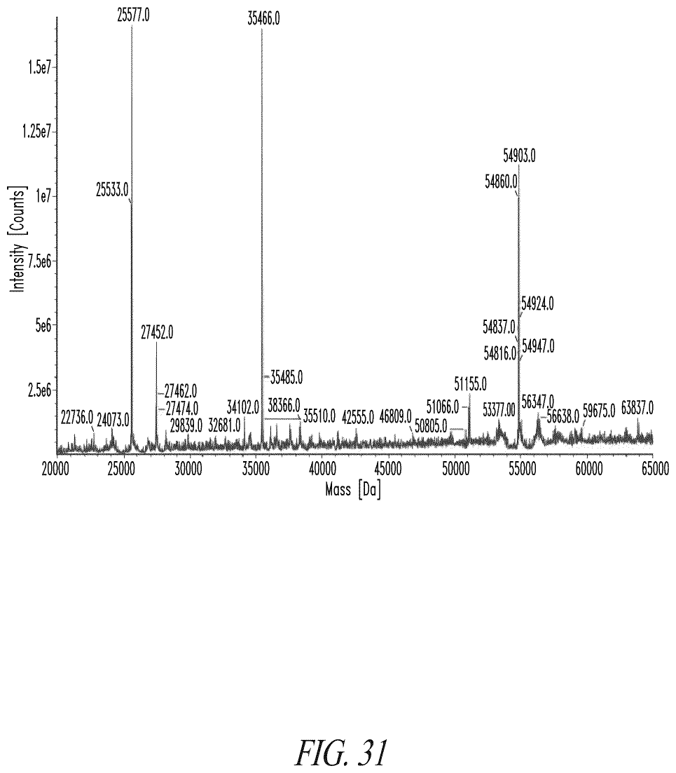

### AV18-P5(PEG24)-VC-PAB-Exatecan DAR8 (FIG. 31)

Calculated LC: 25579, found: 25577 ; Calculated HC: 54950, found: 54947.

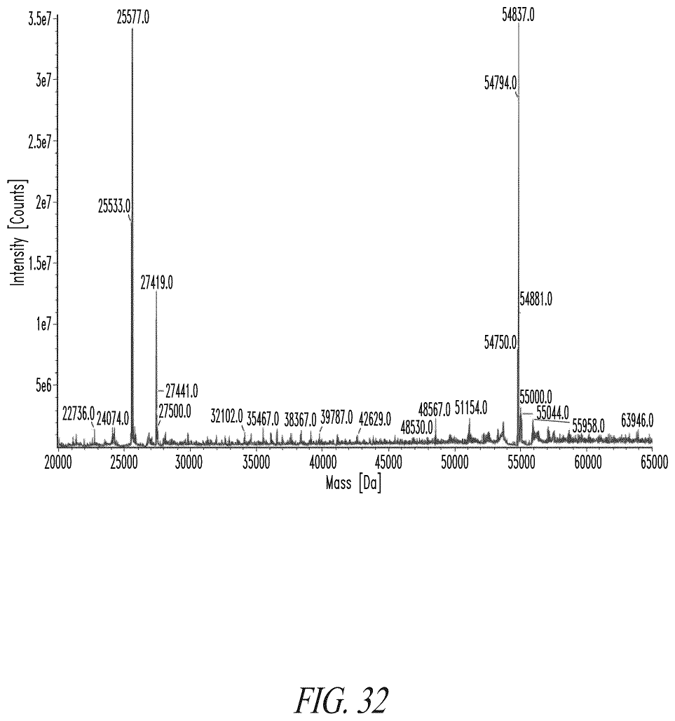

### AV21-P5(PEG24)-VC-PAB-Exatecan DAR8 (FIG. 32)

Calculated LC: 25579, found: 25577; Calculated HC: 54882, found: 54881.

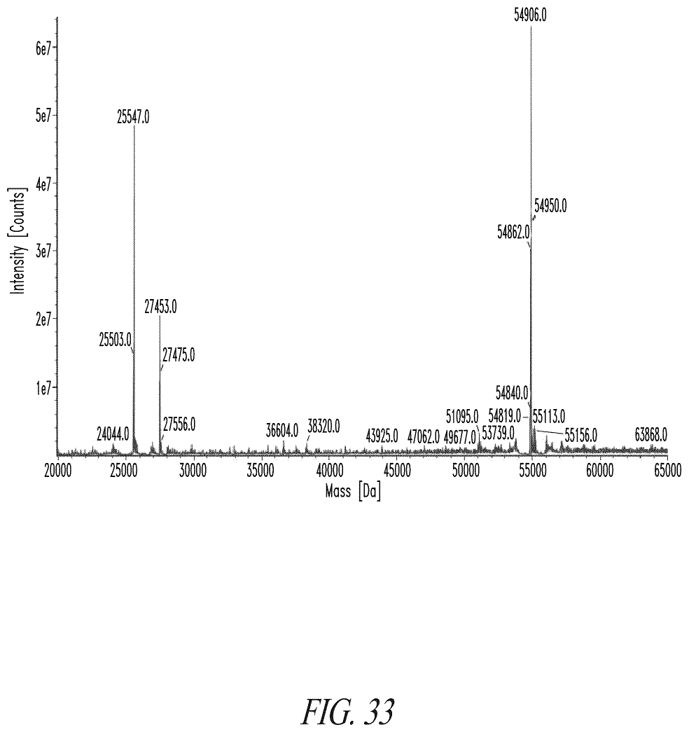

### AV25-P5(PEG24)-VC-PAB-Exatecan DAR8 (FIG. 33)

Calculated LC: 25549, found: 25547; Calculated HC: 54950, found: 54950.

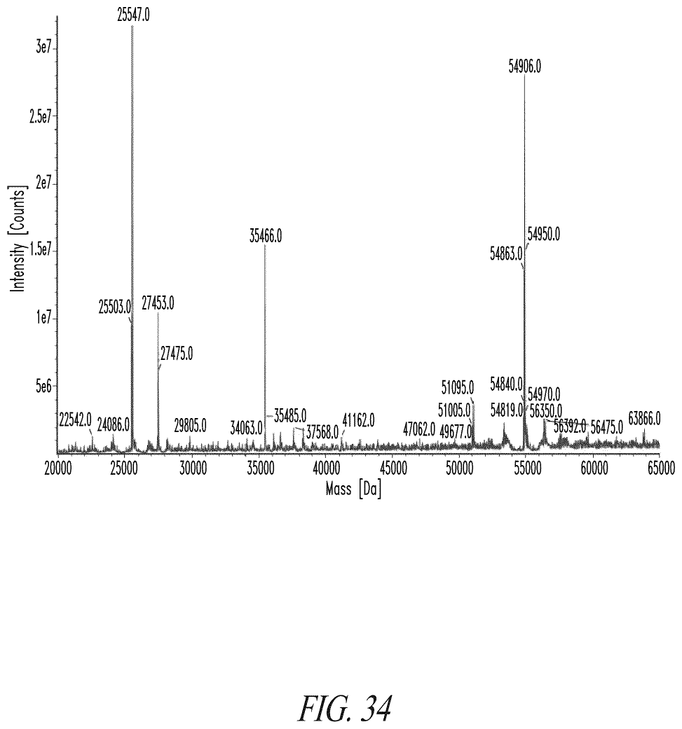

### AV29-P5(PEG24)-VC-PAB-Exatecan DAR8 (FIG. 34)

Calculated LC: 25549 found: 25547; Calculated HC: 54952, found: 54950.

### Example 2: Ex Vivo Serum Stability, In Vivo Efficacy in a Cell Line Derived Xenograft Model (CDX) and In Vivo Efficacy in Patient Derived Xenograft Models (PDX) of an Exemplary ADC of the Present Invention

### Ex Vivo Serum Stability

Serum samples of the respective species were spiked with AV25-P5(PEG24)-VC-PAB-Exatecan DAR 8 at a concentration of 0.2 mg/ml in at least 80% serum. Samples were sterile filtered with UFC3OGVOS centrifugal filter units (Merck, Germany) and incubated at 37° C. for 1, 2, 3, 5 and 7 days. Samples for day 0 were directly processed further.

Recombinant NaPi2B-antigen was coupled to Thermo NHS Magnetic beads according to the manufacturer's instructions. The bead storage solution was removed from 40 μl of the NaPi2B-coupled bead suspension. The beads were incubated with 100 μl of the serum-ADC mix, premixed with 200 μl PBS, for 2h at room temperature. Afterwards, the supernatant was removed and the resin washed 2 times with 1 mL PBS-T. Following by incubation for 15 minutes with 10 μl 100 mM Glycin buffer pH 2.5 at room temperature. This solution was rebuffered to PBS by using 75 μL Zeba™ Spin Desalting Columns with 7K MWCO (Thermo Fisher Scientific, USA). The samples were processed further for MS-measurements, as described above. The Drug-to-Antibody ratio (DAR) was calculated from the MS intensities of the light chain adducts conjugated to 0 or 1 and heavy chain adducts conjugated to 0-3 molecules of P5(PEG24)-VC-PAB-Exatecan.

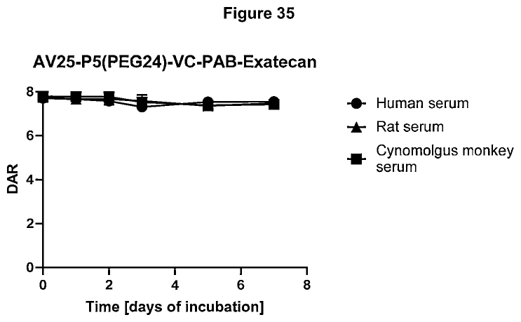

The results clearly show that the linker between the AV25 antibody and the Exatecan drug molecules is highly stable in sera of different species, without a significant amount of payload loss after several days of incubation of the ADC (No change in the Drug-to-Antibody Ratio (DAR) (FIG. 35).

### In Vivo Efficacy of the ADC in a Cell Line Derived Xenograft Model (CDX)

All animal experiments were conducted in accordance with German animal welfare law and approved by local authorities. In brief, 1×10⁷ OVCAR-3 cells (100 μL+100 μL Matrigel) were subcutaneously injected to CB17-Scid mice. Treatment was initiated when tumours reached a mean tumour volume of 0.1-0.15 cm³ 15 days after implantation. 5 animals per group were treated once at day 0 with either 1 mg/kg, 3 mg/kg or 5 mg/kg of AV25-P5(PEG24)-VC-PAB-Exatecan DAR 8, Isotype-P5(PEG24)-VC-PAB-Exatecan DAR 8, or vehicle, as intravenous injection after randomisation into treatment and control groups. Tumour volumes, body weights and general health conditions were recorded throughout the whole study.

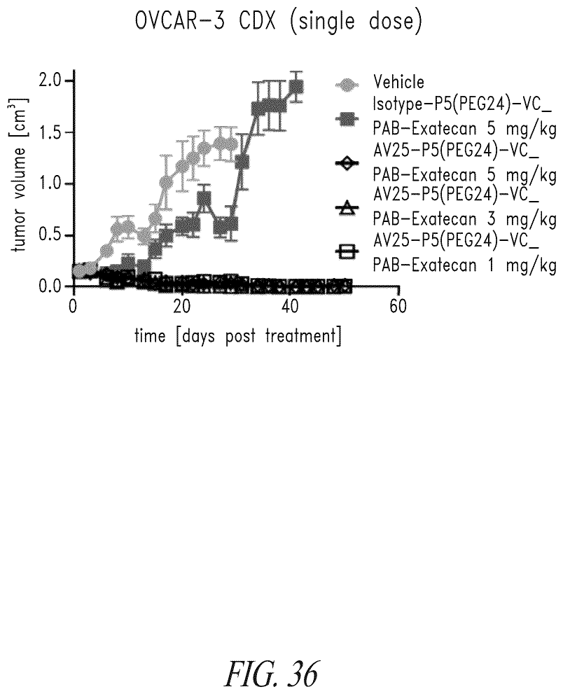

Complete tumor remission over all dose levels (1,3 and 5 mg/kg) at a single injection was observed for the targeted AV25-P5(PEG24)-VC-PAB-Exatecan DAR 8. Whereas only minor effects were observed for the highest dose of 5 mg/kg for the non-targeted isotype control Isotype-P5(PEG24)-VC-PAB-Exatecan DAR 8 (left) (FIG. 36). This clearly shows a marked effect of NaPi2B targeting with AV25-P5(PEG24)-VC-PAB-Exatecan DAR 8. The comparison to literature-known NaPi2B-targeting ADCs such as lifastuzumab vedotin, in in vivo models, derived from the same OVCAR3 cell line, shows a clear advantage for AV25-P5(PEG24)-VC-PAB-Exatecan DAR 8 over all tested dose levels. AV25-P5(PEG24)-VC-PAB-Exatecan DAR 8 does not show any tumor relapse until the end of the observation period with a single dose as low as 1 mg/kg, which is very unusual compared to other NaPi2B targeting ADCs, dosed even higher and more often in the same OVCAR3 model.

### In Vivo Efficacy of the ADC in Patient Derived Xenograft Models (PDX)

All animal experiments were conducted in accordance with German animal welfare law and approved by local authorities. In brief, a 3×3 mm sample of a patient derived tumor sample was subcutaneously implanted into flanks of female immunodeficient NMRI nu/nu mice. Treatment was initiated when tumours reached a mean tumour volume of 0.1-0.15 cm³. Animals per group were treated once at day 0 with 10 mg/kg of AV25-P5(PEG24)-VC-PAB-Exatecan DAR 8 or vehicle, as intravenous injection. Tumour volumes, body weights and general health conditions were recorded throughout the whole study.

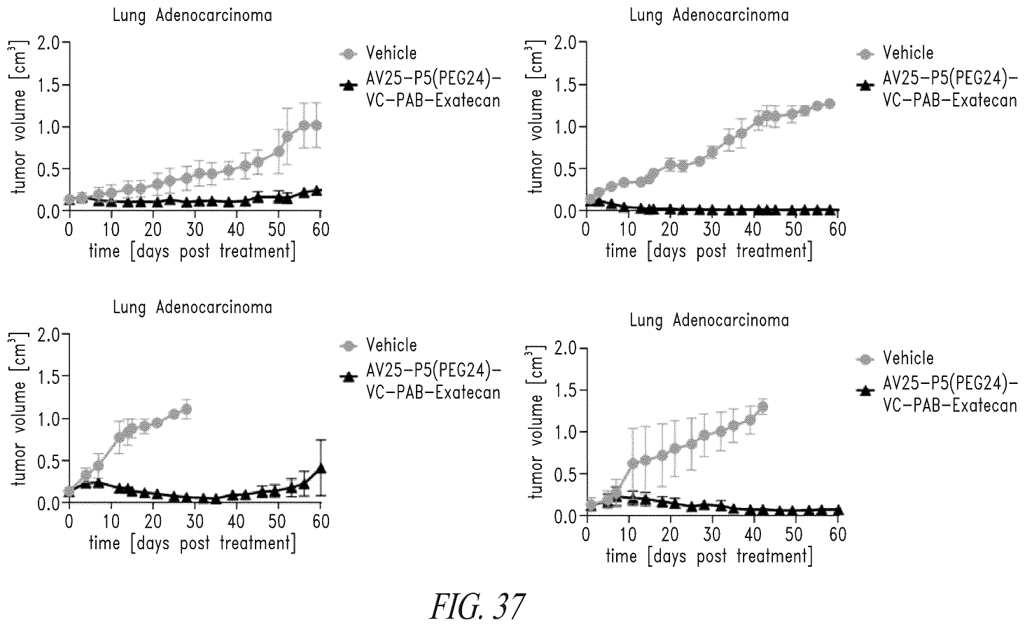

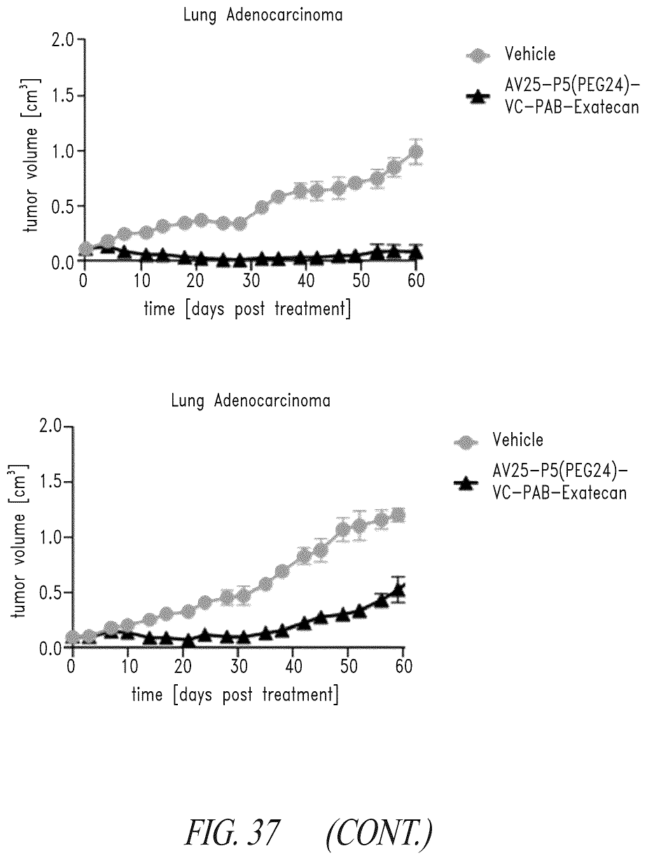

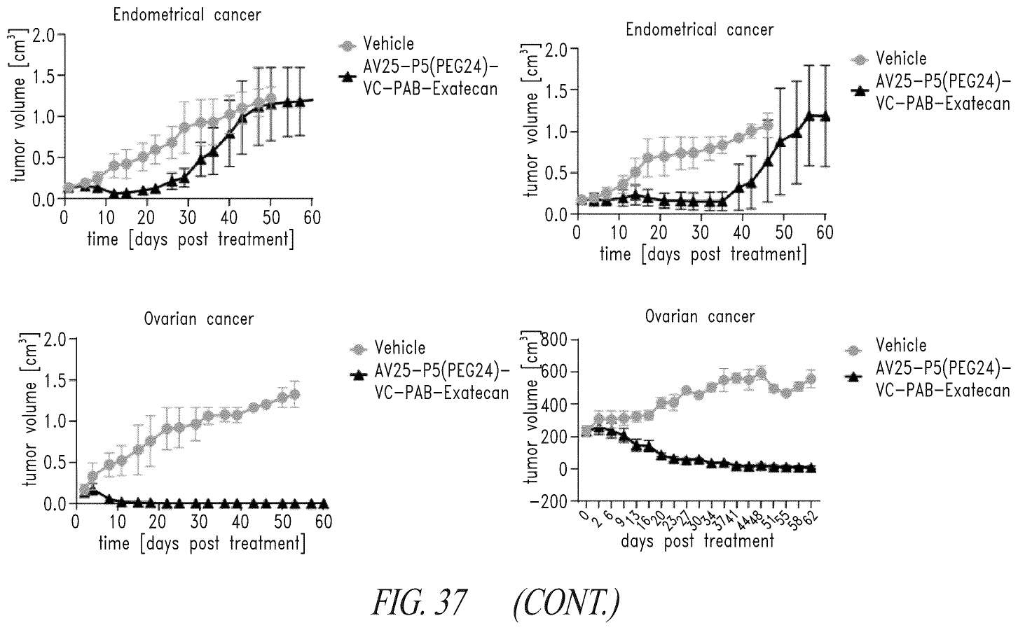

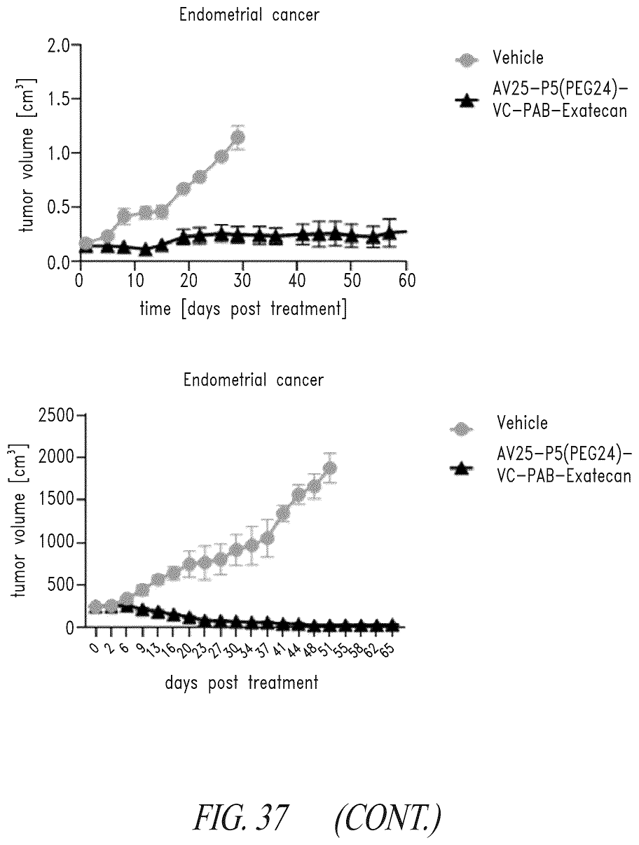

The data clearly demonstrates very high and long-lasting efficacy in patient derived tumor models in vivo compared to the vehicle control in different tumor types such as lung, endometrial and ovarian cancer (FIG. 37).

### In Vivo Toxicity in Cynomolgus Monkeys

Toxicology studies were performed in cynomolgus monkeys. Two animals for each group were dosed intravenously with 10 and 20 mg/kg AV25-P5(PEG24)-VC-PAB-Exatecan on days 1 and 22, with terminal necropsy at day 43. Three different analytes, including total ADC, intact ADC and free Exatecan were analyzed in the animal plasma samples through ELISA and LC/MS-MS. Exposure and pharmacokinetic (PK) parameters were assessed for each analyte. No severe changes in body weight, clinical chemistry, hematology and histopathology were observed at those dose levels. The HNSTD (highest nonseverely toxic dose) was defined to be at least 20 mg/kg in this study.

For PK analysis, both, total ADC and intact ADC were measured via ELISA. The first ELISA setup is for the quantification of the human antibody (total antibody/total mAb).

The first ELISA setup is for the quantification of the human antibody (total antibody/total mAb). For that, recombinant purified NaPi2B antigen is immobilized on protein-binding plates. Samples and standard dilutions are added to the wells and AV25-P5(PEG24)-VC-PAB-Exatecan, independently from its DAR, will be captured. A specific human detection antibody, recognizing the kappa-LC is added to the wells to enable quantification of the captured total antibody.

The second ELISA setup is designed to specifically measure the intact ADC (with the linker-payload attached). For that an anti-payload specific antibody is immobilized on binding plates. Only AV25-P5(PEG24)-VC-PAB-Exatecan with (at least one) conjugated payload molecules are detected. Samples and standard dilutions are added to the wells and only payload-conjugated AV25-P5(PEG24)-VC-PAB-Exatecan will be captured. A specific human detection antibody, recognizing the kappa-LC, is added to the wells to enable quantification of the captured total antibody.

The amount of free Exatecan in plasma samples from cynomolgus monkey after treatments with AV25-P5(PEG24)-VC-PAB-Exatecan, was determined by liquid-chromatography mass spectrometry (LCMS). A quantification method was established on a Xevo G2-XS qTOF machine, using MRM (multiple reaction monitoring) acquisition mode and specific mass transitions for Exatecan. Deuterated Exatecan-d5 was used as internal standard during all measurements. To quantify the amount of free Exatecan in serum samples from different treatment groups and timepoints, a liquid-extraction procedure was performed, separating protein content from lipophilic small molecules. For the preparation of calibration curves and control samples, pre-dilutions of Exatecan and Exatecan-d5 were prepared in ACN:H₂O 1:1, at concentrations of 100 nM and 10 nM, respectively. The assay has been performed in accordance with a quantification assay that was previously described for DxD by Nagai et al., 2018.

The PK analysis shows a good exposure profile, without enhanced in vivo clearance in the cross reactive species (FIG. 38).

The HNSTD (highest nonseverely toxic dose) was defined to be at least 20 mg/kg in this study. This dose is at least 6-times higher compared to doses that were explored in toxicology studies with other NaPi2B-targeting ADCs (FIG. 38).

This significant improvement in increasing tolerability of the ADC and the excellent PK-profile could be attributed to the improved properties of the antibody clones described herein, such as reduced aggregation tendency of the AV25 clone that has been described above. Antibody and ADC aggregation has been shown to be one of the major off-target toxicity drivers of ADCs.

### Example 3: Further Characterization of the AV25 mAb and ADCs Based Thereon

For all data shown, AV25-P5(PEG24)-VC-PAB-Exatecan and AV25 refers to the LALA-mutated version of the AV25 mab, if not explicitly labeled as AV25-wt.

### Synthesis of Anti-Napi2b Comparison Antibody

DNA coding for the light chain
(SEQ ID NO: 56
MLPSQLIGFLLLWVPASRGDIQMTQSPSSLSASVGDRVTITCRSSETLVHSSGNTYLEWYQQKPG
KAPKLLIYRVSNRFSGVPSRFSGSGSGTDFTLTISSLQPEDFATYYCFQGSFNPLTFGQGTKVEIK
RTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC*; LC is shown in italic)
and
heavy chain
(SEQ ID NO: 57
MDWTWRILFLVAAATGAHSEVQLVESGGGLVQPGGSLRLSCAASGFSFSDFAMSWVRQAPGKG
LEWVATIGRVAFHTYYPDSMKGRFTISRDNSKNTLYLQMNSLRAEDTAVYYCARHRGFDVGHFDF
WGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEAA
GGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMH
EALHNHYTQKSLSLSPGK*; HC is shown in italic)

of anti-NaPi2b comparison antibody was synthesized (Geneart, Thermo Fisher). Signal sequences for heavy chain (SEQ ID NO: 58, MDWTWRILFLVAAATGAHS) and light chain (SEQ ID NO: 59, MLPSQLIGFLLLWVPASRG) were added. Light chain and heavy chain sequences were cloned into pcDNA3.4-TOPO (Thermo Fisher) expression plasmid. Antibodies were then transiently expressed in Expi-CHO-S cells (Thermo Fisher) by co-transfecting cells with pcDNA3.4 expression plasmids (Thermo Fisher), coding for the heavy and light chain of the respective sequences in a 1:1 ratio, using the Expi-CHO transfection system (Thermo Fisher). Cells were harvested by centrifugation at 300 g for 5 minutes at 4° C. To clear micro particles from supernatant, supernatants were centrifuged at 4000-5000 g for 30 min at 4° C. For further clarification supernatants were passed through a 0.22 μm filter. Antibodies were purified from cleared and filtered supernatants via Protein A chromatography.

### Synthesis of Anti-Napi2b Comparison ADC

50 μl of a solution of a 10 mg/mL anti-Napi2b comparison antibody (66.67 μM) in Dulbecco's-PBS (Merck KGaA) were mixed with 1.33 μl of a TCEP solution (0.5 mM in buffered solution, Merck KGaA diluted to 10 mM with PBS, 4 eq. TCEP with respect to the antibody). After 30 Min incubation at RT, 1,66 μl of a 20 mM solution of Maleimidocaproyl valine citruilline p-aminobenzyl carbamate-MonomethylauristatinE (MC-VC-PAB-MMAE) in DMSO (10.0 eq. with respect to the antibody) were added. The mixture was shaken (350 RPM) at room temperature (25° C.) for 1 hour. The reaction mixture was purified by preparative size-exclusion chromatography with a 25 ml Superdex™ 200 Increase 10/300GL (Cytiva, Sweden) and a flow of 0.8 ml/min eluting with sterile PBS (Merck, Germany). The antibody containing fractions were pooled and concentrated by spin-filtration (Amicon® Ultra-2mL MWCO: 30 kDa, Merck, Germany) and analyzed by MS, as described above. MS analysis has been performed as described in example 1.

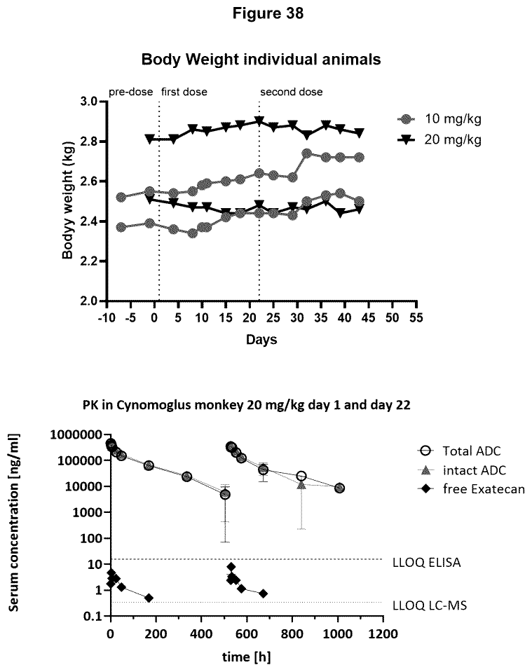

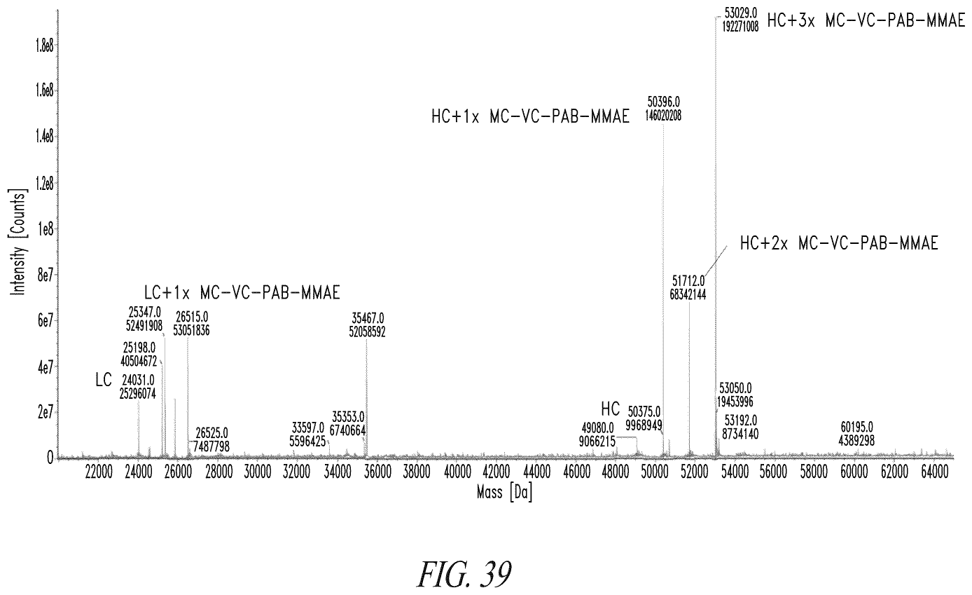

FIG. 39 shows a MS analysis of the anti-Napi2b comparison ADC, synthesized by the method described above. Signals are annotated with the measured mass in Dalton and the absolute intensities. Shown is an exemplary spectrum from the conjugation reaction. The Drug-to-Antibody ratio of the final conjugated have been estimated from the MS signals to 3.5 to 4.0. LC: light chain of anti-Napi2b comparison antibody, HC: heavy chain anti-Napi2b comparison antibody.

### Binding to Human NaPi2a, NaPi2b and NaPi2c Evaluated by Flow Cytometry

To determine concentration-dependent binding, HEK293 cells transiently or stably expressing human Napi2a-, Napi2b- and Napi2c-mCherry were incubated with antibodies or ADCs in concentrations ranging from 0.002 to 200 nM, stained with an Alexa-dye-labeled anti-human IgG H+L secondary antibody (Thermo Fisher Scientific), and analyzed by flow cytometry. Mean fluorescence intensity (MFI) ratios were normalized to the non-specific binding control. The assay was performed in duplicates and data points were analyzed by a non-linear regression using a one-site specific binding model using Prism 9 software. Graph shows means of n=2±SD.

In this experiment, specificity of AV25 and AV25-P5(PEG24)-VC-PAB-Exatecan towards Napi2b was determined. Of note, AV25 only shows binding to HEK293 cells overexpressing Napi2b (SLC34A2), but not Napi2a (SLC34A1) or Napi2c (SLC34A3). Both are members of the SLC34 family sharing 45.5-50.9% sequence homology with Napi2b.

The experiment clearly demonstrates highest selectivity of AV25 to target NaPi2b over the other NaPi2 proteins. This highlights an important feature for the antibody disclosed herein for selective tumor targeting.

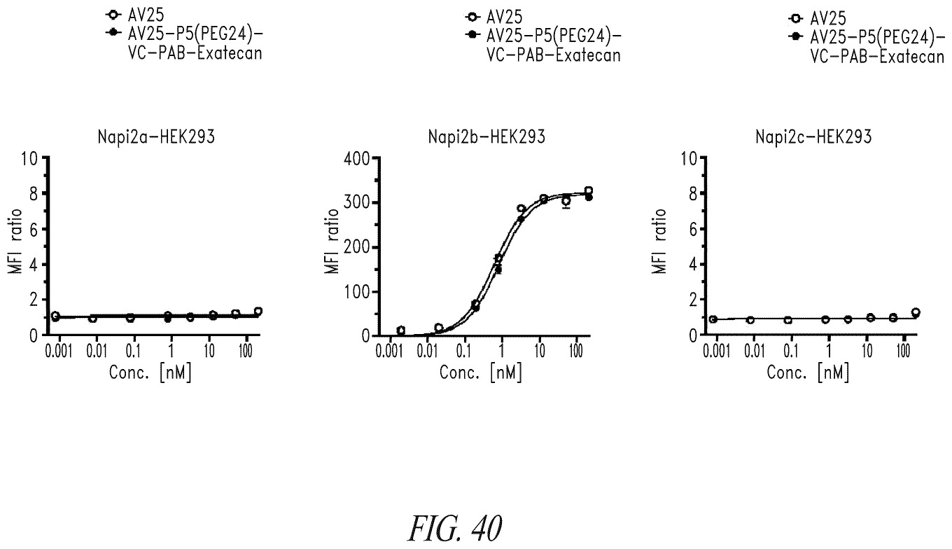

FIG. 40 shows the binding, expressed as MFI ratio, of unmodified AV25 mAb and AV25-P5(PEG24)-VC-PAB-Exatecan DAR8 ADC to HEK293-cells transfected with Napi2a (left), Napi2b (middle) and Napi2c (right). AV25 mAb and AV25-P5(PEG24)-VC-PAB-Exatecan DAR8 ADC bind specifically to NaPi2b. Graphs show n=2±SD (note, error bars are too small to be displayed).

In Vitro Cytotoxicity Evaluated Via Resazurin Assay Synthesis of anti-Napi2b Comparison ADC vs AV25-P5(PEG24)-VC-PAB-Exatecan DAR8

To investigate direct cytotoxicity of ADCs, respective cells were seeded in a 96-well plate (flat bottom, 5000 cells/well, suspended in 100 μl medium) and incubated for 7 days with increasing concentrations of the ADCs in medium (0-12 μg/ml) to generate a dose-response curve. Before viability analysis, the supernatant over the adherent cells was removed and replaced by fresh medium. Killing was analyzed afterwards, using resazurin (Sigma-Aldrich) as the cell viability dye at a final concentration of 55 μM. Fluorescence emission at 590 nM was measured on a Microplate reader Infinite M200 Pro (Tecan). Cell viability was measured by dividing the fluorescence of ADC-treated cells by the fluorescence from control cells, treated in the same way with medium only. Graph shows means of n=2±SEM.

In this experiment, the cytotoxicity on Napi2b^{high}-expressing cell lines of AV25-P5(PEG24)-VC-PAB-Exatecan DAR 8 compared to anti-Napi2b comparison ADC, targeting NaPi2b was investigated. On HCC-78 AV25-P5(PEG24)-VC-PAB-Exatecan showed a tremendous increase in selective potency of a 15.5-fold lower IC50-concetration compared to anti-Napi2b comparison ADC. No unspecific toxicity was observed on target-negative cells.

FIG. 41 shows the cytotoxicity dose-response of AV25-P5(PEG24)-VC-PAB-Exatecan DAR 8 compared to anti-NaPi2b comparison ADC on 3 different cell lines. Shown are mean and SD of two measurements, as well as a dose-response-fit. Fluorescence in % of medium control is equal to % of viable cells.

### In Vitro Inhibition of Topoisomerase-I by the Delivery of Exatecan Via the ADC

Topoisomerase-I inhibition by delivery of exatecan via the AV25-P5(PEG24)-VC-PAB-Exatecan DAR8 ADC induces DNA damage as detected by the accumulation of cleaved PARP, active caspase 3 and phosphorylated histon 2AX (pH2AX). OVCAR-3 and HCC-78 cells (both Napi2b^{high}) were treated with increasing concentrations (0.05-12 μg/ml) of AV25-P5(PEG24)-VC-PAB-Exatecan or an Isotype control (isotype-P5(PEG24)-VC-PAB-Exatecan for 72 h. Cells were stained with live/dead stain (Thermo Fisher Scientific) and after fixation and permeabilization using the Fixation/Permeabilization kit (BD Biosciences) for the DNA damage markers active caspase 3, cleaved PARP and pH2AX (Ser-139) (all BD Biosciences) and analyzed by flow cytometry. Graphs show means of n=2±SEM.

AV25-P5(PEG24)-VC-PAB-Exatecan DAR8 induces a concentration-dependent accumulation of DNA damage as shown by the increase of cleaved PARP-, active caspase 3- and pH2AX-positive HCC-78 (A) and OVCAR-3 (B) cells, whereas the isotype control remains inactive.

The experiment clearly shows that the ADCs are delivering the exatecan selectively into the targeted cell and induce cell killing by topoisomerase-1-inhibition.

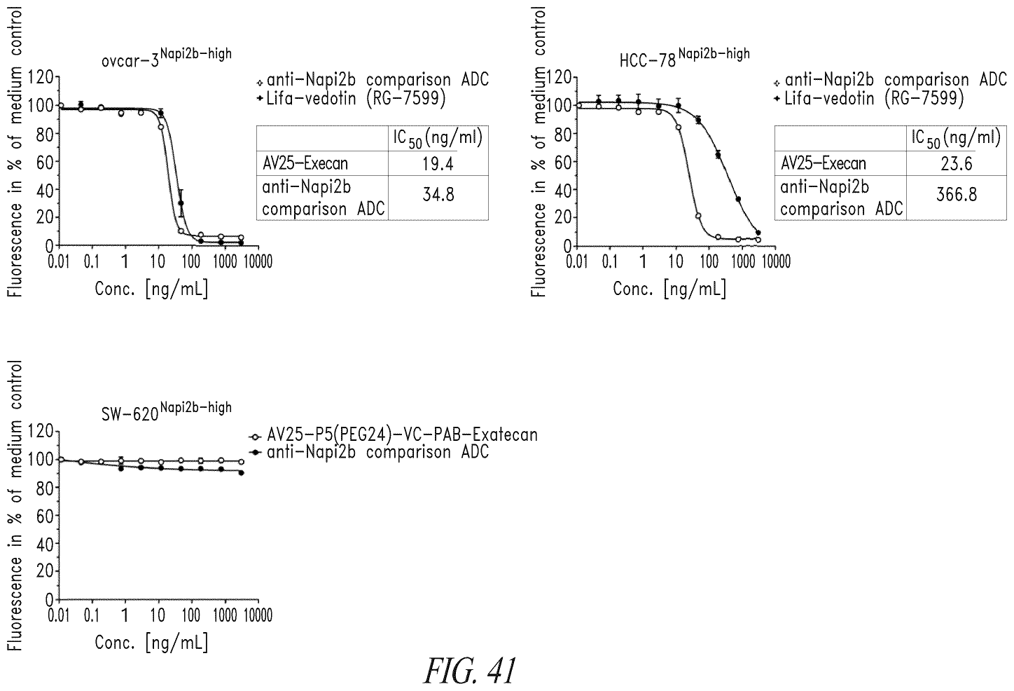

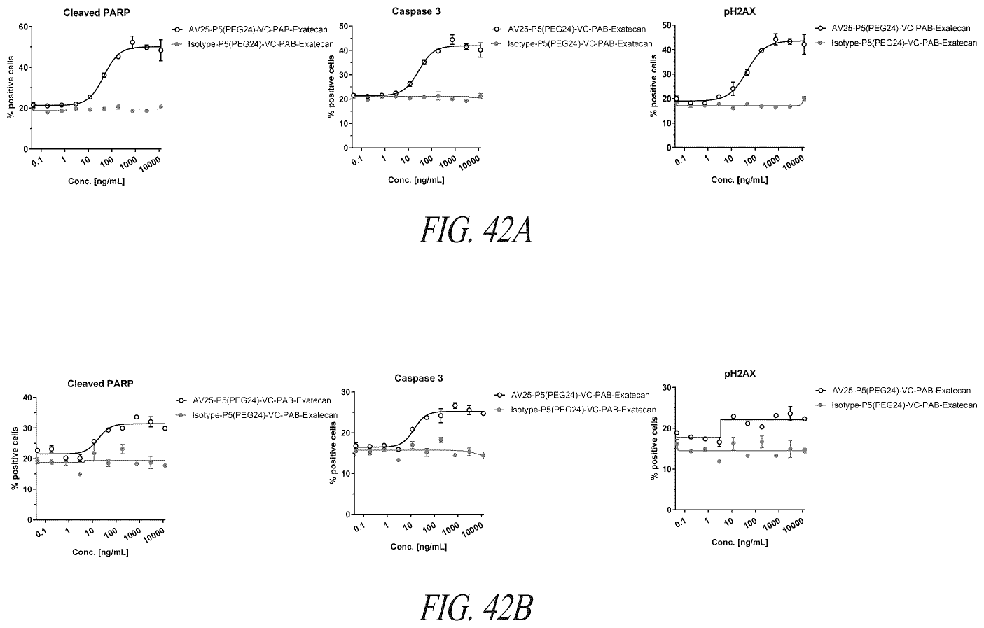

FIGS. 42A and 42B show the dose-dependent induction of DNA-damage and apoptosis markes in response to treatment with increasing concentrations of AV25-P5(PEG24)-VC-PAB-Exatecan DAR8. A corresponding non-targeting isotype control conjugate was included as negative control. HCC-78 cells (A, NaPi2b^{high}) and OVCAR-3 cells (B, NaPi2b^{high}) were treated with increasing concentrations (0.05-12 μg/ml) of AV25-P5(PEG24)-VC-PAB-Exatecan DAR8 or the Isotype ADC (Isotype-P5(PEG24)-VC-PAB-Exatecan DAR8) for 72 h, Cells were stained for cleaved PARP (left), Caspase 3 (middle) and pH2AX (right) and analyzed via flow cytometry. Graphs show means of n=2±SEM.

### Interaction With Complement Factor C1q

To reduce or even prevent interaction of the IgG1 backbone AV25 antibody with complement factors or Fc receptors (FcRs) that might trigger unwanted immune activation and/or FcR-mediated cellular uptake, the Fc-part of the AV25 antibody and the derived ADC AV25-P5(PEG24)-VC-PAB-Exatecan DAR8 has been silenced using two point mutations L234A and L235A (LA LA).

In this experiment, interaction of the LALA-silenced AV25

(HC-LALA: SEQ ID NO: 60
MDWTWRILFLVAAATGAHSEVQLVQSGAEVVKPGASVKMSCKASGYTFTGYIIHW
VKQAPGQGLEWIGAIYPGSGATSYKQKFRGRATLTADTSTSTVYMELSSLRSEDSAVYYCARGET
ARATFAYWGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSCDKTHTCPP
CPAPEAAGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQGNVF
SCSVMHEALHNHYTQKSLSLSPGK*; HC is shown in italic)
vs Fc-wildtype AV25
(HC-wt: SEQ ID NO: 61:
MDWTWRILFLVAAATGAHSEVQLVQSGAEVVKPGASVKMSCKASGYTFTGYIIHWVKQAPGQGL
EWIGAIYPGSGATSYKQKFRGRATLTADTSTSTVYMELSSLRSEDSAVYYCARGETARATFAYWG
QGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGK*; HC is shown in italic)

with complement factor C1q was analyzed. C1q interaction studies were performed using the HTRF Human C1q Binding Kit (Cisbio) according to the manufacturer's instructions. In brief, AV25 HC-wt and HC-LALA, a standard and an anti-MHC-I positive control (Invivogen) known to interact with C1q are captured and aggregated by an anti-human IgG Fab-biotin complexed to streptavidin, which binds streptavidin-labelled d2 (fluorescence acceptor). If the antibodies bind to human C1q, an anti-C1q antibody labelled with Europium cryptate (fluorescence donor) can come in close proximity to the fluorescence acceptor and fluorescence resonance energy transfer (FRET) is triggered. Fluorescence emission at 665 nm is measured on a microplate reader Infinite M200 Pro (Tecan)._Graphs show means of n=2±SD.

While AV25 HC-wt binds similarly well to C1q as the standard and the anti-MHC-I positive control, interaction with C1q is completely abolished for AV25 HC-LALA. The reduced interaction with C1q is expected to decrease undesired activation of the innate immune system.

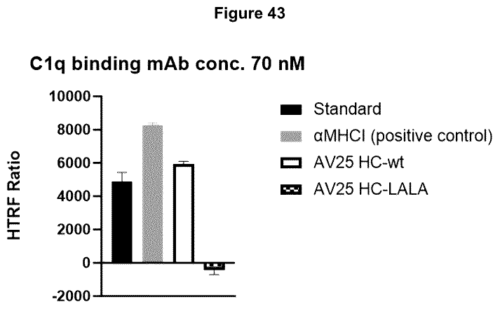

FIG. 43 shows the binding of the Fc region of the AV25 HC-LALA antibody versus the AV25 HC-wt antibody to recombinant hexameric C1q complement protein was measured in a HTRF (Homogenous Time-Resolved Fluorescence) based human C1q binding assay (HTRF Human C1q Binding Kit, Cisbio) according to manufacturer's instructions. In brief, serial dilutions 280 nM-11.6 nM of all tested antibodies (AV25 HC-wt, AV25 HC-LALA, α-MHC-I positive, IgG1 kit standard) were measured. HTRF ratio was calculated by dividing the acceptor emission signal at 665 nm by the donor emission signal at 620 nm and multiplying by 10000. Graph shows HTRF ratio at 70 nM concentration with the background (diluent only) HTRF ratio subtracted. Graph shows means of n=2±SD.

### Interaction With FcRs

To reduce or even prevent interaction of the IgG1 backbone AV25 antibody with complement factors or Fc receptors (FcRs) that might trigger unwanted immune activation and/or FcR-mediated cellular uptake, the Fc-part of the AV25 antibody and the derived ADC AV25-P5(PEG24)-VC-PAB-Exatecan DAR8 has been silenced using two point mutations L234A and L235A (LA LA).

In this experiment, interaction of the LALA-silenced AV25 (HC-LALA: SEQ ID NO. 62:

MDWTWRILFLVAAATGAHSEVQLVQSGAEVVKPGASVKMSCKASGYTFTGYIIHWVKQAPGQGL
EWIGAIYPGSGATSYKQKFRGRATLTADTSTSTVYMELSSLRSEDSAVYYCARGETARATFAYWG
QGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGP
SVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGK*; HC is shown in italic)
and
AV25-P5(PEG24)-VC-PAB-Exatecan DAR8 vs Fc-wildtype AV25
(HC-wt: SEQ ID NO: 63,
MDWTWRILFLVAAATGAHSEVQLVQSGAEVVKPGASVKMSCKASGYTFTGYIIHWVKQAPGQGL
EWIGAIYPGSGATSYKQKFRGRATLTADTSTSTVYMELSSLRSEDSAVYYCARGETARATFAYWG
QGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGK*; HC is shown in italic)

with Fc gamma receptors (FcγRs) was analyzed. FcγR interaction studies were performed using the Lumit™ FcγR Binding Immunoassays (FcγRn, FcγRI, FcγRIIa/CD32 R131/H131 polymorphism, FcγRIIIa/CD16 V158/F158 polymorphism; Promega) based on a competition principle and luciferase detection according to the manufacturer's instructions. In brief, AV25 HC-wt, HC-LALA and AV25-P5(PEG24)-VC-PAB-Exatecan DAR8, a standard and trastuzumab as positive control were incubated with a Tracer-LgBiT and a FcγR-SmBiT. In the absence of an antibody analyte or if no interaction of the tested antibody with FcγRs takes place, Tracer-LgBiT binds to the FcγR-SmBiT target, resulting in maximum luminescence signal. In the case of successful interaction with FcγRs, the tested antibody/ADC will compete with Tracer-LgBiT for binding to the FcγR target, resulting in a concentration-dependent decrease in the luminescent signal. Luminescence is measured on a microplate reader Infinite M200 Pro (Tecan). Graphs show n=1.

While AV25 HC-wt binds similarly well to all FcγR as the IgG1 positive control, interaction with FcγR is either highly reduced or in most cases completely abolished for AV25 HC-LALA and AV25-P5(PEG24)-VC-PAB-Exatecan DAR8. Interestingly, the positive control trastuzumab even shows reduced interaction with all FcγR compared to the positive control. In the FcγRn interaction experiment, the murine AV25 served as another negative control.

The experiment clearly showed reduction of undesired interaction with FcγRI (CD64), CD16 and CD32, mediated by the incorporation of the LALA mutation into the AV25 antibody. The reduced interaction with those receptors is expected to decrease undesired uptake of the AV25 related ADC conjugates into non-targeted cells and thereby reduce undesired toxicities. Interaction with FcRn was only mildly reduced.

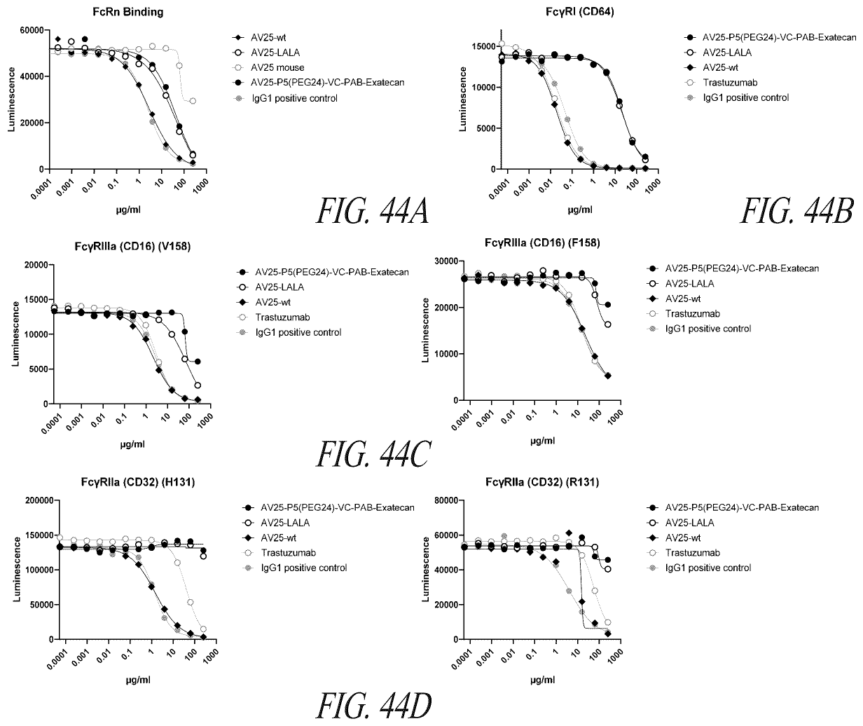

FIGS. 44A to 44D show dose-dependent binding of the Fc region of the AV25-HC-LALA antibody and the AV25-P5(PEG24)-VC-PAB-Exatecan DAR8 ADC versus the AV25-HC-wt antibody to recombinant human FcRn and FcγR was measured using Lumit™ FcγR Binding Immunoassays (FcγRn, FcγRI, FcγRIIa/CD32 R131/H131 polymorphism, FcγRIIa/CD16 V158/F158 polymorphism; Promega) according to the manufacturer's instructions. Serial dilutions of AV25 HC-wt, AV25 HC-LALA and AV25-P5(PEG24)-VC-PAB-Exatecan DAR8, a standard and trastuzumab as positive control were incubated with the Tracer-LgBiT and a FcγR-SmBiT. In the absence of an antibody analyte or if no interaction of the tested antibody with FcγRs takes place, Tracer-LgBiT binds to the FcγR-SmBiT target, resulting in maximum luminescence signal. In the case of successful interaction with FcγRs, the tested antibody/ADC will compete with Tracer-LgBiT for binding to the FcγR target, resulting in a concentration-dependent decrease in the luminescent signal. Luminescence was measured on a microplate reader Infinite M200 Pro (Tecan). Graphs show n=1.

### ADCC

To reduce or even prevent interaction of the IgG1 backbone AV25 antibody with complement factors or Fcγ receptors (FcγRs) that might trigger unwanted immune activation and/or FcR-mediated cellular uptake, the Fc-part of the AV25 antibody and the derived ADC AV25-P5(PEG24)-VC-PAB-Exatecan DAR8 has been silenced using two point mutations L234A and L235A (LA LA).

In this experiment, the antibody-dependent cellular cytotoxicity (ADCC) of the Fc-wildtype AV25 mAb (HC-wt: SEQ ID NO: 64,

MDWTWRILFLVAAATGAHSEVQLVQSGAEVVKPGASVKMSCKASGYTFTGYIIHWVKQAPGQGL
EWIGAIYPGSGATSYKQKFRGRATLTADTSTSTVYMELSSLRSEDSAVYYCARGETARATFAYWG
QGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGK*; HC is shown in italic)
and
LALA-silenced AV25 mAb
(HC-LALA: SEQ ID NO: 65,
MDWTWRILFLVAAATGAHSEVQLVQSGAEVVKPGASVKMSCKASGYTFTGYIIHWVKQAPGQGL
EWIGAIYPGSGATSYKQKFRGRATLTADTSTSTVYMELSSLRSEDSAVYYCARGETARATFAYWG
QGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGP
SVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGK*; HC is shown in italic)

and AV25-P5(PEG24)-VC-PAB-Exatecan DAR8 vs the isotypes and an anti-MHC-I positive control (Invivogen) was analyzed, which is mediated after interaction with FcγRIIIa (CD16). For the Calcein release-based antibody-dependent cellular cytotoxicity (ADCC) assay, peripheral blood mononuclear cells (PBMCs) were isolated from healthy donor buffy coats (purchased from DONAS GmbH) using LeucoSep tubes (Greiner Bio-One) and Histopaque®-1077 (density 1.077 g/ml, Merck) density gradient according to standard protocols. Afterwards, natural killer (NK) cells were MACS (magnetic cell separation)-sorted using human NK cell isolation kit (Miltenyi Biotec) by negative selection according to the manufacturer's instructions yielding untouched human primary NK cells. Napi2b-positive target cells OVCAR-3 and HCC-78 (both Napi2b^{high}) were stained with 16 μM Calcein AM (Thermo Fisher Scientific). 40.000 NK cells and 10.000 Calcein-stained target cells were incubated at a 4:1 ratio in the presence of 15 μg/ml antibodies or ADCs. Cells permeabilized with 2.5% Triton X 100 (Merck) served as a positive control for maximum Calcein release. After 4 h, supernatants were transferred to a flat black non-binding 96-well plate (Greiner Bio-One) and fluorescence was measured at 485/535 nM via the Infinite M200 Pro reader (Tecan). The percent specific killing was calculated by dividing the Calcein released by antibody-mediated killing minus background Calcein release (NKs+targets) from Calcein released by Triton X-permeabilized cells (maximum killing) minus background Calcein release (targets only). Graphs show means of n=2±SEM.

The anti-MHC-I positive control and AV25 HC-wt triggered high ADCC activity in human NK cells, whereas all LALA-silenced antibodies and ADCs (AV25 HC-LALA, AV25-P5(PEG24)-VC-PAB-Exatecan DAR8 and isotype controls) did not induce ADCC or only in very low amounts. Reduced ADCC is expected to decrease undesired toxicities of AV25 related antibodies.

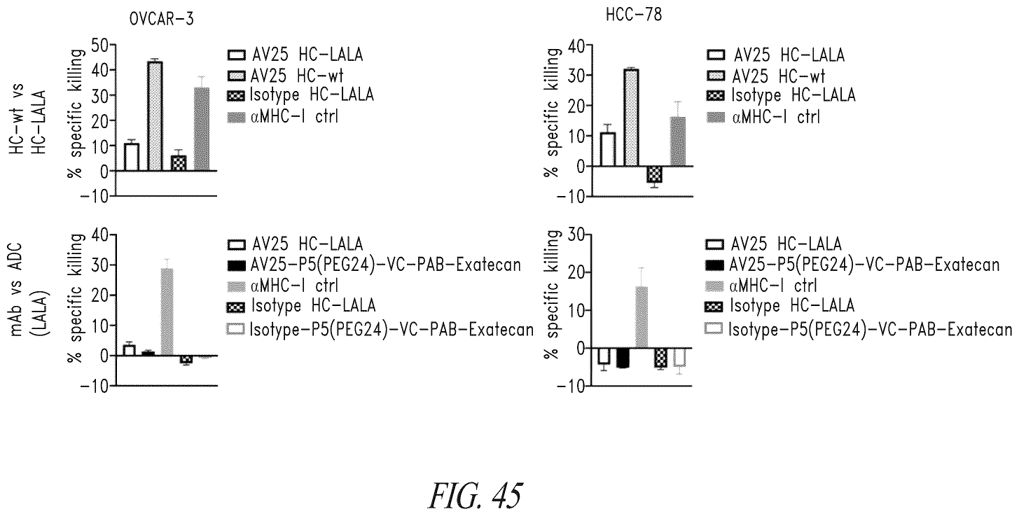

FIG. 45 shows a calcein release-based antibody-dependent cellular cytotoxicity (ADCC) assay. Co-cultures of healthy donor (HD)-derived NK cells and calcein-stained target-positive tumor cells (OVCAR-3 and HCC-78), in a 4:1 ratio, were incubated with 15 μg/ml of indicated antibodies or ADCs (anti-MHC-I antibody served as positive control). The percentage of specific killing was calculated by deviding the calcein released by antibody-mediated cell killing from calcein released by Triton X-permeabilized cells (maximum killing). Graphs show means of n=2±SEM.

### AV25-P5(PEG24)-VC-PAB-Exatecan DAR8 Dose Response and Exposure PDX Study (Lu7700)

All animal experiments were conducted in accordance with German animal welfare law and approved by local authorities. In brief, a 3×3 mm sample of a patient derived tumor sample (Lu7700 non-small cell lung cancer (NSCLC) model, EPO Experimentelle Pharmakologie & Onkologie Berlin-Buch GmbH) was subcutaneously implanted into flanks of female immunodeficient NMRI nu/nu mice. Treatment was initiated when tumours reached a mean tumour volume of 0.1-0.15 cm³. Animals per group were treated once at day 0 with 5, 3 or 1 mg/kg of AV25-P5(PEG24)-VC-PAB-Exatecan DAR 8, 5 mg/kg isotype-P5(PEG24)-VC-PAB-Exatecan DAR 8 or vehicle, as intravenous injection. Tumour volumes, body weights and general health conditions were recorded throughout the whole study.

The data demonstrate highest and long-lasting efficacy for all tested ADC dose levels in the NSCLC PDX models in vivo compared to the vehicle control. The effect is highly specific for the targeted anti-TPBG-antibody, exemplified by the non-targeted isotype control ADC group at the highest dose.

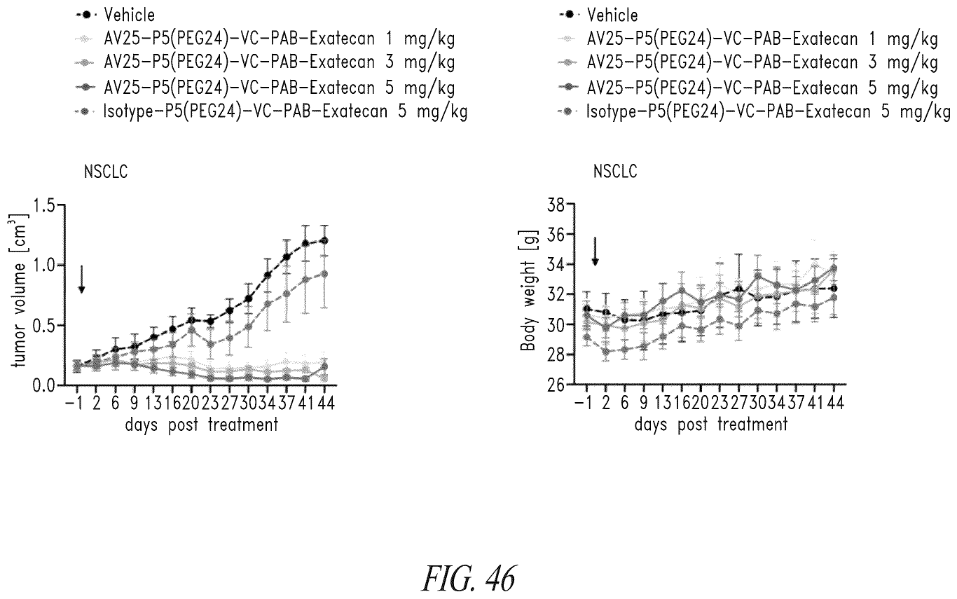

FIG. 46 shows the results of an in vivo efficacy analysis of the AV25-P5(PEG24)-VC-PAB-Exatecan (DAR 8), which is a representative ADC of the present invention, in a patient-derived NSCLC xenograft model (PDX, Lu7700). Shown on the left is tumor volume over time after treatment once at day 0 with different dose levels of the AV25-P5(PEG24)-VC-PAB-Exatecan (DAR 8) (1, 3 and 5 mg ADC/kg bodyweight), an isotype control carrying the same amount of linker-payload (5 mg/kg) versus untreated (vehicle). Shown on the right are bodyweights of the animals treated with different dose levels of the AV25-P5(PEG24)-VC-PAB-Exatecan (DAR 8), an isotype control carrying the same amount of linker-payload versus untreated (vehicle). All results are shown as Mean and SEM of 4 animals per group.

AV25-P5(PEG24)-VC-PAB-Exatecan DAR8 Dose Response and Exposure PDX Study (0v6668)

All animal experiments were conducted in accordance with German animal welfare law and approved by local authorities. In brief, a 3×3 mm sample of a patient derived tumor sample (0v6668 ovarian cancer model, EPO Experimentelle Pharmakologie & Onkologie Berlin-Buch GmbH) was subcutaneously implanted into flanks of female immunodeficient NMRI nu/nu mice. Treatment was initiated when tumours reached a mean tumour volume of 0.1-0.15 cm³. Animals per group were treated once at day 0 with 5, 3 or 1 mg/kg of AV25-P5(PEG24)-VC-PAB-Exatecan DAR 8 or vehicle, as intravenous injection. Tumour volumes, body weights and general health conditions were recorded throughout the whole study.

The data demonstrate highest and long-lasting efficacy for all tested ADC dose levels in the PDX models in vivo compared to the vehicle control. Dose-proportionality for exposure has been demonstrated in the PK data. The PK analysis has been performed as described in example 1.

Complete tumor remission with dose levels of 5 and 3 mg/kg in a ovarian cancer PDX at a single injection was observed for the targeted AV25-P5(PEG24)-VC-PAB-Exatecan DAR 8. The 1 mg/kg dosing in the ovarian cancer model also showed strong tumor regression which was followed by partial tumor re-growth after day 40. The PK analysis shows dose proportional exposure profiles and high stability of the AV25-P5(PEG24)-VC-PAB-Exatecan DAR 8 ADC at the tested dose levels 5, 3 and 1 mg/kg.

The data demonstrate high and long-lasting efficacy for all tested ADC dose levels of AV25-P5(PEG24)-VC-PAB-Exatecan DAR 8 in the PDX model in vivo compared to the vehicle. No reduction in bodyweight suggests highest tolerability. The PK analysis shows dose proportional exposure profiles and high stability of the AV25-P5(PEG24)-VC-PAB-Exatecan DAR 8 ADC at the tested dose levels 5, 3 and 1 mg/kg.

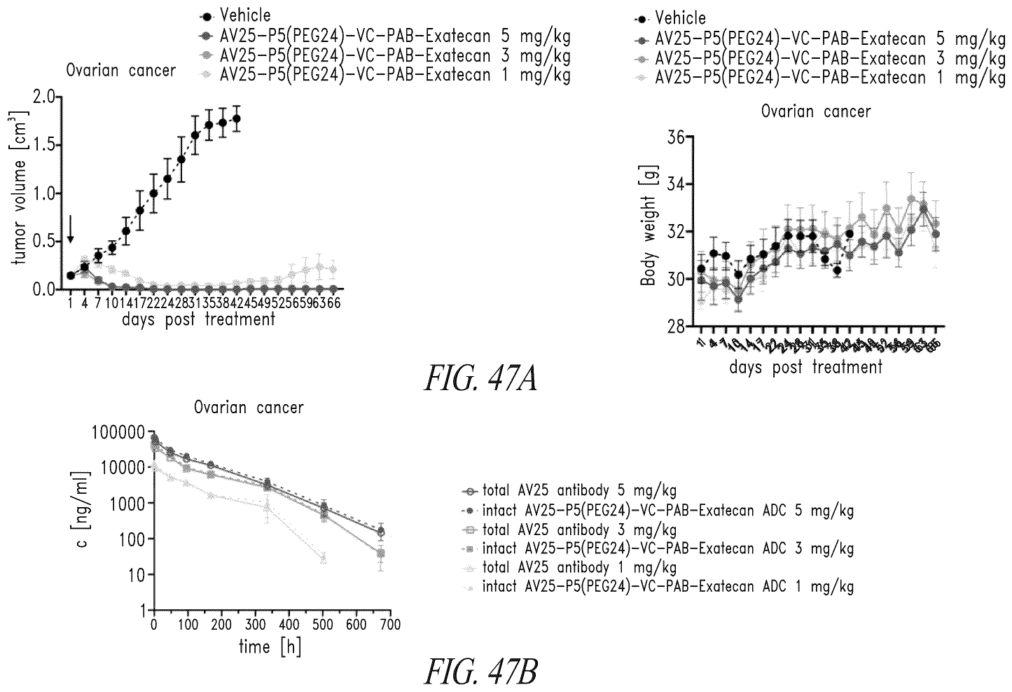

FIG. 47A shows the results of an in vivo efficacy analysis of the AV25-P5(PEG24)-VC-PAB-Exatecan DAR 8, which is a representative ADC of the present invention, in a patient-derived ovarian cancer xenograft model (PDX, Ov6668). Shown on the left is tumor volume over time after treatment once at day 0 with different dose levels of the AV25-P5(PEG24)-VC-PAB-Exatecan (DAR 8) (1, 3 and 5 mg ADC/kg bodyweight) versus untreated (vehicle). Shown on the right are bodyweights of the animals treated with different dose levels of the AV25-P5(PEG24)-VC-PAB-Exatecan (DAR 8), versus untreated (vehicle). All results are shown as Mean and SEM of 9 animals per group. FIG. 47B shows the in vivo PK evaluation for total and intact ADC for the three dose levels of the dose-response efficacy study as Mean and SD from three measurements per time point from mice treated once at day 0 with different dose levels of the AV25-P5(PEG24)-VC-PAB-Exatecan (1, 3 and 5 mg ADC/kg bodyweight). The PK analysis has been performed as described in example 1.

AV25-P5(PEG24)-VC-PAB-Exatecan DAR8 PDX Studies (10 mg/kg)

The figure shows results of an in vivo efficacy analysis of a single 10 mg/kg dose of either AV25-P5(PEG24)-VC-PAB-Exatecan (DAR 8), which is a representative ADC of the present invention, or isotype-AV25-P5(PEG24)-VC-PAB-Exatecan (DAR 8) in three patient-derived ovarian cancer xenograft models with various NaPi2b expression levels and BRCA mutational states (PDX). The PDX experiments have been performed as described in example 2.

The data clearly demonstrates very high and long-lasting efficacy in patient derived ovarian cancer tumor models in vivo compared to the vehicle control, even in very difficult to treat BRCA-mutated PDX models and models that exhibit low Napi2b target expression.

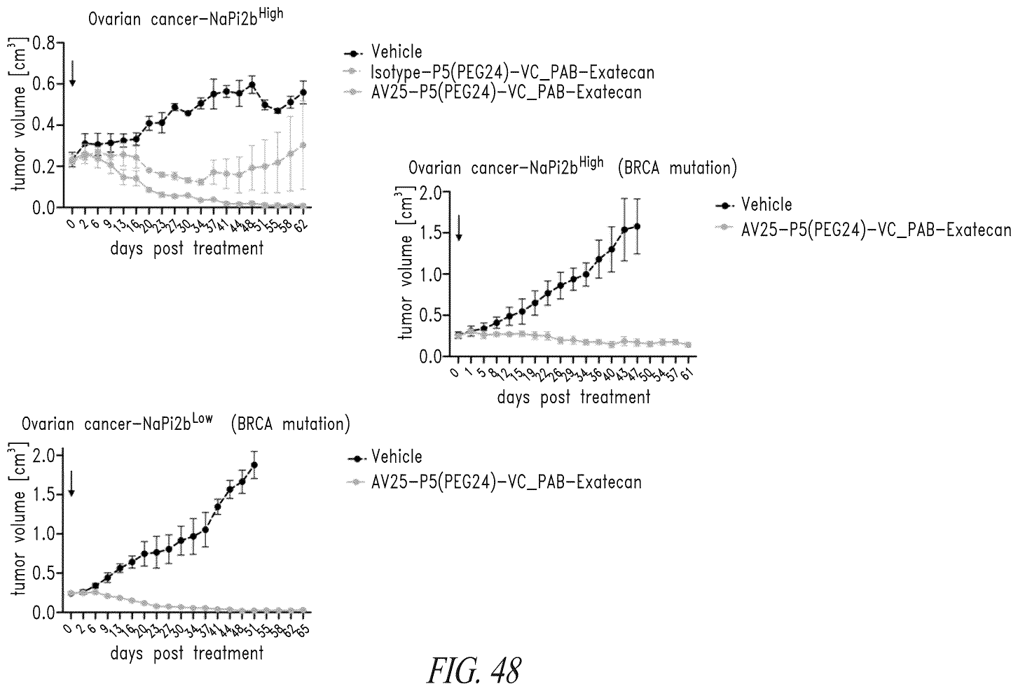

FIG. 48 shows the results of an in vivo efficacy analysis of the AV25-P5(PEG24)-VC-PAB-Exatecan (DAR 8), which is a representative ADC of the present invention, in patient-derived ovarian cancer xenograft models. Shown on top is tumor volume over time after treatment once at day 0 with 10 mg ADC/kg bodyweight of the AV25-P5(PEG24)-VC-PAB-Exatecan (DAR 8) or Isotype-P5(PEG24)-VC-PAB-Exatecan (DAR 8) versus untreated (vehicle). Shown on the bottom is a BRCA-mutated model with low NaPi2B expression (bottom left) and high NaPi2B expression (bottom right). All results are shown as Mean and SEM of 3 animals per group.

### Binding to NaPi2b-Expressing Cell Lines OVCAR-3 and HCC-78 Evaluated by Flow Cytometry—Upifitamab vs AV25

Upifitamab is antibody with the same sequence of the parental mAb of the present disclosure. Upifitamab has been purchased from MedChemExpressed to perform the following experiments.

To determine concentration-dependent binding, NaPi2b-positive OVCAR-3 and HCC-78 were incubated with antibodies in concentrations ranging from 0.00075 to 30 μg/ml, stained with an Alexa-dye-labeled anti-human IgG H+L secondary antibody (Thermo Fisher Scientific), and analyzed by flow cytometry. Mean fluorescence intensity (MFI) ratios were normalized to the non-specific binding control. The assay was performed in duplicates and data points were analyzed by a non-linear regression using a one-site specific binding model using Prism 9 software. Graph shows means of n=2±SD.

In this experiment, the binding between AV25 and upifitamab on NaPi2b-positive cell lines was compared and K_{D} values were determined. Of note, AV25 (K_{D} on OVCAR-3=0.44 μg/ml, K_{D} on HCC-78 0.42 μg/ml) shows improved binding and lower K_{D} values compared to upifitamab (K_{D} on OVCAR-3 1.11 μg/ml, K_{D} on HCC-78 1.27 μg/ml).

The experiment clearly demonstrates the superiority in binding of AV25 over Upifitamab.

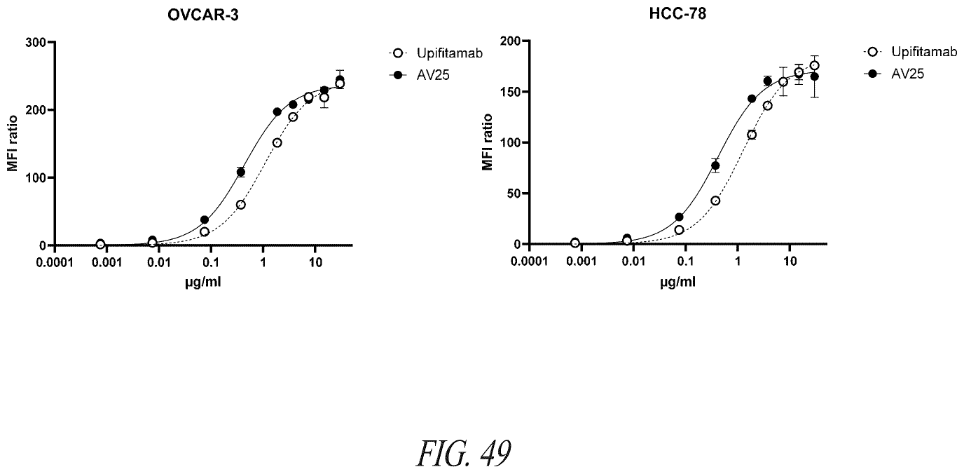

FIG. 49 shows dose-dependent binding, expressed as MFI ratio, of increasing concentraions of Upifitamab or AV25 normalized to the non-specific binding control on two different NaPi2b-positive cell-lines (OVCAR-3, left, HCC78, right). Shown are mean and SD of two measurements, as well as a dose-response-fit.

In vitro Cytotoxicity Evaluated Via Resazurin Assay—Upifitamab-P5(PEG24)-VC-PAB-Exatecan vs AV25-P5(PEG24)-VC-PAB-Exatecan DAR8

Upifitamab is antibody with the same sequence of the parental mAb of the present disclosure. Upifitamab has been purchased from MedChemExpressed to perform the following experiments. Upifitamab has been furthermore conjugated to P5(PEG24)-VC-PAB-Exatecan and purified as DAR8 conjugate as described above in example 1.

To investigate direct cytotoxicity of ADCs, respective cells were seeded in a 96-well plate (flat bottom, 5000 cells/well, suspended in 100 μl medium) and incubated for 7 days with increasing concentrations of the ADCs in medium (0-12 μg/ml) to generate a dose-response curve. Before viability analysis, the supernatant over the adherent cells was removed and replaced by fresh medium. Killing was analyzed afterwards, using resazurin (Sigma-Aldrich) as the cell viability dye at a final concentration of 55 μM. Fluorescence emission at 590 nM was measured on a Microplate reader Infinite M200 Pro (Tecan). Cell viability was measured by dividing the fluorescence of ADC-treated cells by the fluorescence from control cells, treated in the same way with medium only. Graph shows means of n=2±SEM.

In this experiment, the cytotoxicity on Napi2b^{high}-expressing cell lines (OVCAR-3 and HCC-78) of AV25-P5(PEG24)-VC-PAB-Exatecan DAR 8 compared head-to-.head to Upifitamab-P5(PEG24)-VC-PAB-Exatecan DAR 8 was investigated. On both cell lines AV25-P5(PEG24)-VC-PAB-Exatecan showed an increase in selective potency of a 3.5 to 3.8-fold lower IC50-concetration compared to Upifitamab-P5(PEG24)-VC-PAB-Exatecan. No unspecific toxicity of the non-targeted isotype control was observed.

The experiment clearly demonstrates the superiority in killing of AV25-P5(PEG24)-VC-PAB-Exatecan over upifitamab-P5(PEG24)-VC-PAB-Exatecan suggesting an increase in response in the cancer patient to a targeted treatment based on a targeting AV25 antibody, compared to the parental mAb or Upifitamab.

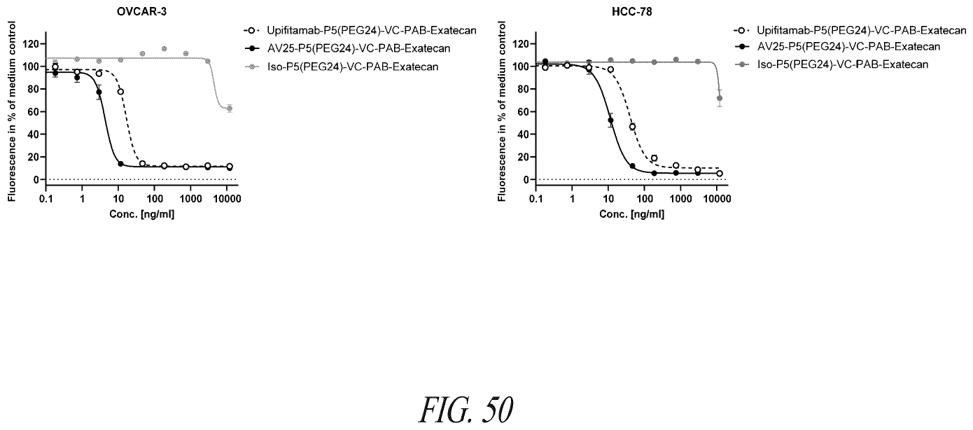

FIG. 50 shows the cytotoxicity dose-response of AV25-P5(PEG24)-VC-PAB-Exatecan DAR 8 compared to Upifitamab-P5(PEG24)-VC-PAB-Exatecan DAR 8 and an isotype control Isotype-P5(PEG24)-VC-PAB-Exatecan DAR 8 on two different cell lines. Shown are mean and SD of two measurements, as well as a dose-response-fit. Fluorescence in % of medium control is equal to % of viable cells.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. An anti-NaPi2b antibody, wherein said NaPi2b antibody is capable of the following:
a) binding to human Napi2b and/or rat Napi2b;
b) cross-reactivity with rat Napi2b;
c) cross-reactivity with cynomolgus monkey;
d) internalization, optionally by the means of the antigen-mediated antibody internalization; and
e) not having a dipeptide deamidation site in a heavy chain variable region's (V_{H}) CDR2, optionally said absent dipeptide deamidation site is NG (Asn-Gly) in V_{H} CDR2.

2. The anti-NaPi2b antibody according to claim 1, wherein said anti-NaPi2b antibody is an antibody comprising:
a) a heavy chain variable region having an amino acid sequence with at least 80% identity to SEQ ID NO: 4 and a light chain variable region having an amino acid sequence with at least 80% identity to SEQ ID NO: 5; optionally said anti-NaPi2b antibody is the AV25 antibody comprising:a light chain comprising SEQ ID NO: 6 and a heavy chain comprising SEQ ID NO: 7;
b) a heavy chain variable region having an amino acid sequence with at least 80% identity to SEQ ID NO: 8 and a light chain variable region having an amino acid sequence with at least 80% identity to SEQ ID NO: 9; optionally said anti-NaPi2b antibody is the AV-15 antibody comprising: a light chain comprising SEQ ID NO: 10 and a heavy chain comprising SEQ ID NO: 11;
c) a heavy chain variable region having an amino acid sequence with at least 80% identity to SEQ ID NO: 12 and a light chain variable region having an amino acid sequence with at least 80% identity to SEQ ID NO: 13; optionally said anti-NaPi2b antibody is the AV-18 antibody comprising: a light chain comprising SEQ ID NO: 14 and a heavy chain comprising SEQ ID NO: 15;
d) a heavy chain variable region having an amino acid sequence with at least 80% identity to SEQ ID NO: 16 and a light chain variable region having an amino acid sequence with at least 80% identity to SEQ ID NO: 17; optionally said anti-NaPi2b antibody is the AV-21 antibody comprising: a light chain comprising comprising SEQ ID NO: 18 and a heavy chain comprising comprising SEQ ID NO: 19;
e) a heavy chain variable region having an amino acid sequence with at least 80% identity to SEQ ID NO: 20 and a light chain variable region having an amino acid sequence with at least 80% identity to SEQ ID NO: 21; optionally said anti-NaPi2b antibody is the AV-29 antibody comprising: a light chain comprising comprising SEQ ID NO: 22 and and a heavy chain comprising comprising SEQ ID NO: 23.

3. The anti-NaPi2b antibody according to claim 1, wherein said anti-NaPi2b antibody is:
a) an antibody comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26 and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29;
b) an antibody comprising a heavy chain variable region comprising heavy chain CDR1 having the amino acid sequence as set forth in SEQ ID NO: 30, heavy chain CDR2 having the amino acid sequence as set forth in SEQ ID NO: 31, and heavy chain CDR3 having the amino acid sequence as set forth in SEQ ID NO: 32 and a light chain variable region comprising light chain CDR1 having the amino acid sequence as set forth in SEQ ID NO: 33, light chain CDR2 having the amino acid sequence as set forth in SEQ ID NO: 34, and light chain CDR3 having the amino acid sequence as set forth in SEQ ID NO: 35;
c) an antibody comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38 and a light chain variable region comprising light chain CDR1 having the amino acid sequence as set forth in SEQ ID NO: 39, light chain CDR2 having the amino acid sequence as set forth in SEQ ID NO: 40, and light chain CDR3 having the amino acid sequence as set forth in SEQ ID NO: 41;
d) an antibody comprising a heavy chain variable region comprising heavy chain CDR1 having the amino acid sequence as set forth in SEQ ID NO: 42, heavy chain CDR2 having the amino acid sequence as set forth in SEQ ID NO: 43, and heavy chain CDR3 having the amino acid sequence as set forth in SEQ ID NO: 44 and a light chain variable region comprising light chain CDR1 having the amino acid sequence as set forth in SEQ ID NO: 45, light chain CDR2 having the amino acid sequence as set forth in SEQ ID NO: 46, and light chain CDR3 having the amino acid sequence as set forth in SEQ ID NO: 47;
e) an antibody comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50 and a light chain variable region comprising light chain CDR1 having the amino acid sequence as set forth in SEQ ID NO: 51, light chain CDR2 having the amino acid sequence as set forth in SEQ ID NO: 52, and light chain CDR3 having the amino acid sequence as set forth in SEQ ID NO: 53.

4. The anti-NaPi2b antibody according to claim 1, wherein said anti-NaPi2b antibody has one or more of the following characteristics:
a) a monoclonal antibody;
b) a chimeric antibody and/or humanized anbibody;
c) specifically recognizes Napi2b overexpressed on cancer cells;
d) human IgG antibody, optionally a human IgG1 antibody;
e) comprising kappa (κ) light chain;
f) comprising lambda (λ) light chain;
g) comprising Fc silencing mutations such as leucine (L) to alanine (A) substitution at the position 234 and 235 (LALA mutations), wherein said LALA mutations are capable of reducing immunocell effector function/s;
h) capable of being internalized by target cells expressing Napi2b; optionally said internalized antibody is directed to lysosomes;
i) a tumor-selective antibody, optionally said tumor is a liquid and/or solid tumor;
j) a malignant-cell selective antibody;
k) binding to said human and/or rat Napi2b in a glycosylation-dependent manner, wherein said antibody binds to a glycosylated form of said Napi2b protein;
l) having K_{D} to an endogenously expressed human Napi2b in the range from about 0.01 to about 10 nmol/L, optionally in the range from about 1 to about 10 nmol/L, further optionally from about 1 to about 7 nmol/L, further optionally from about 1 to about 4 nmol/L, further optionally said K_{D} is measured by the means of a FACS assay, further optionally having said K_{D} in the range from about 2.661 to about 6.644 nmol/L;
m) optionally, having K_{D} in the range from about 0.01 to about 10 nmol/L to an immobilized exogenous full-length Napi2b and/or one or more fragments thereof;
n) cross-reactivity with rat Napi2b; and/or
o) not having a dipeptide deamidation site in a heavy chain variable region's (V_{H}) CDR2, optionally said absent dipeptide deamidation site is NG (Asn-Gly) in V_{H}CDR2.

5. A nucleic acid encoding the antibody according to claim 1.

6. An antibody drug conjugate (ADC) comprising the anti-NaPi2b antibody according to claim 1.

7. A composition or kit comprising said anti-NaPi2b antibody of claim 1.

8. The composition according to claim 5, wherein said composition is a pharmaceutical and/or diagnostic composition.

9. A method of producing an antibody drug conjugate (ADC), said method comprising:
conjugating the antibody according to claim 1 to one or more cytotoxic moieties.

10. A method for treatment, amelioration, prophylaxis and/or diagnostics of cancer, said method comprising: administering a therapeutically or prophylactically effective amount of the antibody according to claim 1.


SEQUENCE LISTING (65 sequences)

SEQ ID NO: 1 - protein, 690 aa, Homo sapiens
  REGION 1..690: note = MISC_FEATURE - Sodium-dependent phosphate transport protein 2B having UniProtAccession Number: O95436
  DOMAIN 122..135: note = Extracellular Domain
  DOMAIN 234..362: note = Extracellular Domain
  DOMAIN 429..485: note = Extracellular Domain
  DOMAIN 547..552: note = Extracellular Domain
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL

SEQ ID NO: 2 - protein, 695 aa, Rattus norvegicus
  REGION 1..695: note = MISC_FEATURE - Sodium-dependent phosphate transport protein 2B having UniProtAccession Number: Q9JJ09
MAPWPELENAHPNPNKFIEGASGPQSSIPDKDKGTSKTNDSGTPVAKIELLPSYSALVLIEEPPEGNDPWDLPELQDNGIKWSERDSKGKILCIFQGIGKFILLLGFLYLFVCSLDVLSSAFQLVGGKMAGQFFSNNSIMSNPVAGLVIGVLVTVMVQSSSTSSSIIVSMVASSLLSVRAAIPIIMGANIGTSITNTIVALMQAGDRNEFRRAFAGATVHDFFNWLSVLVLLPLEAATHYLEKLTNLVLETFSFQNGEDAPDILKVITDPFTKLIIQLDKKVIQQIAMGDSEAQNKSLIKIWCKTISNVIEENVTVPSPDNCTSPSYCWTDGIQTWTIQNVTEKENIAKCQHIFVNFSLPDLAVGIILLTVSLLILCGCLIMIVKLLGSVLRGQVATVIKKTLNTDFPFPFAWLTGYLAILVGAGMTFIVQSSSVFTSAMTPLIGIGVISIERAYPLTLGSNIGTTTTAILAALASPGNTLRSSLQIALCHFFFNISGILLWYPIPFTRLPIRLAKGLGNISAKYRWFAVFYLIFFFLLTPLTVFGLSLAGWPVLVGVGVPIILLILLVLCLRMLQARCPRILPLKLRDWNFLPLWMHSLKPWDNIISLATSCFQRRCCCCCRVCCRVCCMVCGCKCCRCSKCCKNLEEEEKEQDVPVKASGGFDNTAMSKECQDEGKGQVEVLGMKALSNTTVF

SEQ ID NO: 3 - protein, 690 aa, Macaca fascicularis
  REGION 1..690: note = MISC_FEATURE - Sodium-dependent phosphate transport protein 2B having UniProtKBAccession Number: A0A2K5UHY1
MAPWPELGDAQPNPSKYLEGATSQQPITPDKSKETNKTDNTEVPVTKFELLPTYSTATLIEEPTEVDDPWNLPTLQDSGIKWSERDAKGKILCVFQGIGRLILLLGFLYFFVCSLDVLSSAFQLVGGKMAGQFFSNSSIMSNPLLGLVIGVLVTVFVQSSSTSTSIVVSMVASSLLTVRAAIPIIMGANIGTSITNTIVALMQVGDRSEFRRAFAGATVHDFFNWLSVLVLLPVEVATHYLEVVTQLIVESFHFKNGEDAPDLLKVITKPFTKLIVQLDKKVISQIAMNDETAKNKSLVKIWCKTFTNMTQMNVTVPSMANCTSHSLCWTDGIQIWTMKNVTYKENIAKCQHIFVNFHLPDLAVGIILLIISLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNTLRSSLQIALCHFFFNISGILLWYPIPFTRLPIRMAKGLGNISAKYRWFAVFYLIIFFFLIPLTVFGLSLAGWPVLVGVGVPVVFIIILVLCLRLLQSRCPRVLPKKLQNWNFLPLCMHSLKPWDAVISKFTGPFQMPCCCCCRVCCRVCCLLCGCPKCCRCSKCCQDLEEGQEAQGVPVKAPETFDNITISREAQGEVRAPDSKTECTAL

SEQ ID NO: 4 - protein, 119 aa, synthetic construct
  REGION 1..119: note = Heavy chain variable region without a signal seqeunce
EVQLVQSGAEVVKPGASVKMSCKASGYTFTGYIIHWVKQAPGQGLEWIGAIYPGSGATSYKQKFRGRATLTADTSTSTVYMELSSLRSEDSAVYYCARGETARATFAYWGQGTLVTVSS

SEQ ID NO: 5 - protein, 107 aa, synthetic construct
  REGION 1..107: note = Light chain variable region without a signal seqeunce
DIQMTQSPSSLSASVGDRVTITCSASQDIGNFLNWYQQKPGKTVKVLIYYASSLYSGVPSRFSGSGSGTDYTLTISSLQPEDFATYYCQQYSKLPLTFGQGTKLELK

SEQ ID NO: 6 - protein, 214 aa, synthetic construct
  REGION 1..214: note = Light chain without a signal seqeunce
DIQMTQSPSSLSASVGDRVTITCSASQDIGNFLNWYQQKPGKTVKVLIYYASSLYSGVPSRFSGSGSGTDYTLTISSLQPEDFATYYCQQYSKLPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 449 aa, synthetic construct
  REGION 1..449: note = Heavy chain without a signal seqeunce
EVQLVQSGAEVVKPGASVKMSCKASGYTFTGYIIHWVKQAPGQGLEWIGAIYPGSGATSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 119 aa, synthetic construct
  REGION 1..119: note = Heavy chain variable region without a signal seqeunce
EVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSS

SEQ ID NO: 9 - protein, 107 aa, synthetic construct
  REGION 1..107: note = Light chain variable region without a signal seqeunce
DIQMTQSPSSLSASVGDRVTITCSASQDIGKFLNWYQQKPGKTVKVLIYYSSSLYSGVPSRFSGSGSGTDYTLTISSLQPEDFATYYCQQYSKVPLTFGQGTKLELK

SEQ ID NO: 10 - protein, 214 aa, synthetic construct
  REGION 1..214: note = Light chain without a signal seqeunce
DIQMTQSPSSLSASVGDRVTITCSASQDIGKFLNWYQQKPGKTVKVLIYYSSSLYSGVPSRFSGSGSGTDYTLTISSLQPEDFATYYCQQYSKVPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
  REGION 1..449: note = Heavy chain without a signal seqeunce
EVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 119 aa, synthetic construct; identical to SEQ ID NO: 4
  REGION 1..119: note = Heavy chain variable region without a signal seqeunce

SEQ ID NO: 13 - protein, 107 aa, synthetic construct
  REGION 1..107: note = Light chain variable region without a signal seqeunce
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELK

SEQ ID NO: 14 - protein, 214 aa, synthetic construct
  REGION 1..214: note = Light chain without a signal seqeunce
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, synthetic construct; identical to SEQ ID NO: 7
  REGION 1..449: note = Heavy chain without a signal seqeunce

SEQ ID NO: 16 - protein, 119 aa, synthetic construct
  REGION 1..119: note = Heavy chain variable region without a signal seqeunce
EVQLVQSGAEVVKPGASVKMSCKASGYTFTGQIIHWVKQAPGQGLEWIGAISPGNSGTSYKQKFRGRATLTADTSTSTVYMELSSLRSEDSAVYYCARGETARATFAYWGQGTLVTVSS

SEQ ID NO: 17 - protein, 107 aa, synthetic construct; identical to SEQ ID NO: 13
  REGION 1..107: note = Light chain variable region without a signal seqeunce

SEQ ID NO: 18 - protein, 214 aa, synthetic construct; identical to SEQ ID NO: 14
  REGION 1..214: note = Light chain without a signal seqeunce

SEQ ID NO: 19 - protein, 449 aa, synthetic construct
  REGION 1..449: note = Heavy chain without a signal seqeunce
EVQLVQSGAEVVKPGASVKMSCKASGYTFTGQIIHWVKQAPGQGLEWIGAISPGNSGTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 119 aa, synthetic construct
  REGION 1..119: note = Heavy chain variable region without a signal seqeunce
EVQLVQSGAEVVKPGASVKMSCKASGYTFTGYNILWVKQAPGQGLEWIGAIYPGNGGTSYKQKFRGRATITADTSTSTVYMELSSLRSEDSAVYYCARGEIARATFAYWGQGTLVTVSS

SEQ ID NO: 21 - protein, 107 aa, synthetic construct; identical to SEQ ID NO: 5
  REGION 1..107: note = Light chain variable region without a signal seqeunce

SEQ ID NO: 22 - protein, 214 aa, synthetic construct; identical to SEQ ID NO: 6
  REGION 1..214: note = Light chain without a signal seqeunce

SEQ ID NO: 23 - protein, 449 aa, synthetic construct
  REGION 1..449: note = Heavy chain without a signal seqeunce
EVQLVQSGAEVVKPGASVKMSCKASGYTFTGYNILWVKQAPGQGLEWIGAIYPGNGGTSYKQKFRGRATITADTSTSTVYMELSSLRSEDSAVYYCARGEIARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 5 aa, synthetic construct
  REGION 1..5: note = CDR-H1
GYIIH

SEQ ID NO: 25 - protein, 17 aa, synthetic construct
  REGION 1..17: note = CDR-H2
AIYPGSGATSYKQKFRG

SEQ ID NO: 26 - protein, 10 aa, synthetic construct
  REGION 1..10: note = CDR-H3
GETARATFAY

SEQ ID NO: 27 - protein, 11 aa, synthetic construct
  REGION 1..11: note = CDR-L1
SASQDIGNFLN

SEQ ID NO: 28 - protein, 7 aa, synthetic construct
  REGION 1..7: note = CDR-L2
YASSLYS

SEQ ID NO: 29 - protein, 9 aa, synthetic construct
  REGION 1..9: note = CDR-L3
QQYSKLPLT

SEQ ID NO: 30 - protein, 5 aa, synthetic construct
  REGION 1..5: note = CDR-H1
GYNIH

SEQ ID NO: 31 - protein, 17 aa, synthetic construct
  REGION 1..17: note = CDR-H2
AIYPGNGDTSYKQKFRG

SEQ ID NO: 32 - protein, 10 aa, synthetic construct
  REGION 1..10: note = CDR-H3
GETARATFAY

SEQ ID NO: 33 - protein, 11 aa, synthetic construct
  REGION 1..11: note = CDR-L1
SASQDIGKFLN

SEQ ID NO: 34 - protein, 7 aa, synthetic construct
  REGION 1..7: note = CDR-L2
YSSSLYS

SEQ ID NO: 35 - protein, 9 aa, synthetic construct
  REGION 1..9: note = CDR-L3
QQYSKVPLT

SEQ ID NO: 36 - protein, 5 aa, synthetic construct
  REGION 1..5: note = CDR-H1
GYIIH

SEQ ID NO: 37 - protein, 17 aa, synthetic construct
  REGION 1..17: note = CDR-H2
AIYPGSGATSYKQKFRG

SEQ ID NO: 38 - protein, 10 aa, synthetic construct
  REGION 1..10: note = CDR-H3
GETARATFAY

SEQ ID NO: 39 - protein, 11 aa, synthetic construct
  REGION 1..11: note = CDR-L1
SASQDIGNFLN

SEQ ID NO: 40 - protein, 7 aa, synthetic construct
  REGION 1..7: note = CDR-L2
YTSSLYS

SEQ ID NO: 41 - protein, 9 aa, synthetic construct
  REGION 1..9: note = CDR-L3
QQYSKLPLT

SEQ ID NO: 42 - protein, 5 aa, synthetic construct
  REGION 1..5: note = CDR-H1
GQIIH

SEQ ID NO: 43 - protein, 17 aa, synthetic construct
  REGION 1..17: note = CDR-H2
AISPGNSGTSYKQKFRG

SEQ ID NO: 44 - protein, 10 aa, synthetic construct
  REGION 1..10: note = CDR-H3
GETARATFAY

SEQ ID NO: 45 - protein, 11 aa, synthetic construct
  REGION 1..11: note = CDR-L1
SASQDIGNFLN

SEQ ID NO: 46 - protein, 7 aa, synthetic construct
  REGION 1..7: note = CDR-L2
YTSSLYS

SEQ ID NO: 47 - protein, 9 aa, synthetic construct
  REGION 1..9: note = CDR-L3
QQYSKLPLT

SEQ ID NO: 48 - protein, 10 aa, synthetic construct
  REGION 1..10: note = CDR-H1
GYTFTGYNIL

SEQ ID NO: 49 - protein, 17 aa, synthetic construct
  REGION 1..17: note = CDR-H2
AIYPGNGGTSYKQKFRG

SEQ ID NO: 50 - protein, 10 aa, synthetic construct
  REGION 1..10: note = CDR-H3
GEIARATFAY

SEQ ID NO: 51 - protein, 11 aa, synthetic construct
  REGION 1..11: note = CDR-L1
SASQDIGNFLN

SEQ ID NO: 52 - protein, 7 aa, synthetic construct
  REGION 1..7: note = CDR-L2
YASSLYS

SEQ ID NO: 53 - protein, 9 aa, synthetic construct
  REGION 1..9: note = CDR-L3
QQYSKLPLT

SEQ ID NO: 54 - protein, 449 aa, synthetic construct; identical to SEQ ID NO: 11
  REGION 1..449: note = Parental heavy chain without a signal sequence

SEQ ID NO: 55 - protein, 214 aa, synthetic construct; identical to SEQ ID NO: 14
  REGION 1..214: note = Parental light chain without a signal seqeunce

SEQ ID NO: 56 - protein, 238 aa, synthetic construct
MLPSQLIGFLLLWVPASRGDIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 469 aa, synthetic construct
MDWTWRILFLVAAATGAHSEVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 19 aa, synthetic construct
MDWTWRILFLVAAATGAHS

SEQ ID NO: 59 - protein, 19 aa, synthetic construct
MLPSQLIGFLLLWVPASRG

SEQ ID NO: 60 - protein, 468 aa, synthetic construct
MDWTWRILFLVAAATGAHSEVQLVQSGAEVVKPGASVKMSCKASGYTFTGYIIHWVKQAPGQGLEWIGAIYPGSGATSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 468 aa, synthetic construct
MDWTWRILFLVAAATGAHSEVQLVQSGAEVVKPGASVKMSCKASGYTFTGYIIHWVKQAPGQGLEWIGAIYPGSGATSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 468 aa, synthetic construct; identical to SEQ ID NO: 60

SEQ ID NO: 63 - protein, 468 aa, synthetic construct; identical to SEQ ID NO: 61

SEQ ID NO: 64 - protein, 468 aa, synthetic construct; identical to SEQ ID NO: 61

SEQ ID NO: 65 - protein, 468 aa, synthetic construct; identical to SEQ ID NO: 60